SEQUENCE LISTING (48 sequences)

SEQ ID NO: 1 - DNA, 10697 bp, synthetic construct
  misc_feature 1..10697: note = Synthetic polynucleotide
  source 1..10697: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 2 - DNA, 11355 bp, synthetic construct
  misc_feature 1..11355: note = Synthetic polynucleotide
  source 1..11355: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacctgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 3 - DNA, 11420 bp, synthetic construct
  misc_feature 1..11420: note = Synthetic polynucleotide
  source 1..11420: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttgggggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacctgacccaggggactcaatcagcctcgaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtatgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 4 - DNA, 11171 bp, synthetic construct
  misc_feature 1..11171: note = Synthetic polynucleotide
  source 1..11171: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcaccgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 5 - DNA, 11309 bp, synthetic construct
  misc_feature 1..11309: note = Synthetic polynucleotide
  source 1..11309: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaacgagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 6 - DNA, 11293 bp, synthetic construct
  misc_feature 1..11293: note = Synthetic polynucleotide
  source 1..11293: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaactgattgtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatgatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 7 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 8 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactattagatctgatggccgcgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 9 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 10 - DNA, 10700 bp, synthetic construct
  misc_feature 1..10700: note = Synthetic polynucleotide
  source 1..10700: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 11 - DNA, 11188 bp, synthetic construct
  misc_feature 1..11188: note = Synthetic polynucleotide
  source 1..11188: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactattagatctgatggccgcgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 12 - DNA, 11187 bp, synthetic construct
  misc_feature 1..11187: note = Synthetic polynucleotide
  source 1..11187: mol_type = other DNA
ctagaaccggtgacgtctcccatggtgaagcttggatctgaattcggtacctagttataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggtt

SEQ ID NO: 13 - DNA, 10960 bp, synthetic construct
  misc_feature 1..10960: note = Synthetic polynucleotide
  source 1..10960: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacctttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 14 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 15 - DNA, 1608 bp, Homo sapiens
  source 1..1608: mol_type = other DNA
atggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacag

SEQ ID NO: 16 - protein, 524 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 17 - DNA, 1572 bp, Homo sapiens
  source 1..1572: mol_type = other DNA
atgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaac

SEQ ID NO: 18 - protein, 478 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 19 - DNA, 1434 bp, Homo sapiens
  source 1..1434: mol_type = other DNA
atgggccgctgctgcttctacaccgccggcaccctgagcctgctgctgctggtgaccagcgtgaccctgctggtggcccgcgtgttccagaaggccgtggaccagagcatcgagaagaagatcgtgctgcgcaacggcaccgaggccttcgacagctgggagaagccccccctgcccgtgtacacccagttctacttcttcaacgtgaccaaccccgaggagatcctgcgcggcgagaccccccgcgtggaggaggtgggcccctacacctaccgcgagctgcgcaacaaggccaacatccagttcggcgacaacggcaccaccatcagcgccgtgagcaacaaggcctacgtgttcgagcgcgaccagagcgtgggcgaccccaagatcgacctgatccgcaccctgaacatccccgtgctgaccgtgatcgagtggagccaggtgcacttcctgcgcgagatcatcgaggccatgctgaaggcctaccagcagaagctgttcgtgacccacaccgtggacgagctgctgtggggctacaaggacgagatcctgagcctgatccacgtgttccgccccgacatcagcccctacttcggcctgttctacgagaagaacggcaccaacgacggcgactacgtgttcctgaccggcgaggacagctacctgaacttcaccaagatcgtggagtggaacggcaagaccagcctggactggtggatcaccgacaagtgcaacatgatcaacggcaccgacggcgacagcttccaccccctgatcaccaaggacgaggtgctgtacgtgttccccagcgacttctgccgcagcgtgtacatcaccttcagcgactacgagagcgtgcagggcctgcccgccttccgctacaaggtgcccgccgagatcctggccaacaccagcgacaacgccggcttctgcatccccgagggcaactgcctgggcagcggcgtgctgaacgtgagcatctgcaagaacggcgcccccatcatcatgagcttcccccacttctaccaggccgacgagcgcttcgtgagcgccatcgagggcatgcaccccaaccaggaggaccacgagaccttcgtggacatcaaccccctgaccggcatcatcctgaaggccgccaagcgcttccagatcaacatctacgtgaagaagctggacgacttcgtggagaccggcgacatccgcaccatggtgttccccgtgatgtacctgaacgagagcgtgcacatcgacaaggagaccgccagccgcctgaagagcatgatcaacaccaccctgatcatcaccaacatcccctacatcatcatggccctgggcgtgttcttcggcctggtgttcacctggctggcctgcaagggccagggcagcatggacgagggcaccgccgacgagcgcgcccccctgatccgcacc

SEQ ID NO: 20 - DNA, 23 bp, synthetic construct
  misc_feature 1..23: note = Synthetic polynucleotide
  source 1..23: mol_type = other DNA
tggaagacttcgagatacactgt

SEQ ID NO: 21 - DNA, 23 bp, synthetic construct
  misc_feature 1..23: note = Synthetic polynucleotide
  source 1..23: mol_type = other DNA
acagtgtatctcgaagtcttcca

SEQ ID NO: 22 - DNA, 21 bp, synthetic construct
  misc_feature 1..21: note = Synthetic polynucleotide
  source 1..21: mol_type = other DNA
tttagaaataagtggtagtca

SEQ ID NO: 23 - DNA, 21 bp, synthetic construct
  misc_feature 1..21: note = Synthetic polynucleotide
  source 1..21: mol_type = other DNA
tgactaccacttatttctaaa

SEQ ID NO: 24 - DNA, 19 bp, synthetic construct
  misc_feature 1..19: note = Synthetic polynucleotide
  source 1..19: mol_type = other DNA
agggtatcaagactacgaa

SEQ ID NO: 25 - DNA, 19 bp, synthetic construct
  misc_feature 1..19: note = Synthetic polynucleotide
  source 1..19: mol_type = other DNA
ttcgtagtcttgataccct

SEQ ID NO: 26 - DNA, 19 bp, synthetic construct
  misc_feature 1..19: note = Synthetic polynucleotide
  source 1..19: mol_type = other DNA
tattagatctgatggccgc

SEQ ID NO: 27 - DNA, 20 bp, synthetic construct
  misc_feature 1..20: note = Synthetic polynucleotide
  source 1..20: mol_type = other DNA
ctccatcactaggggttcct

SEQ ID NO: 28 - DNA, 60 bp, synthetic construct
  misc_feature 1..60: note = Synthetic polynucleotide
  source 1..60: mol_type = other DNA
agctctgggtatttaagcccgagtgagcacgcagggtctccattttgaagcgggaggtta

SEQ ID NO: 29 - DNA, 145 bp, unidentified
  misc_feature 1..145: note = AAV2 ITR
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 30 - DNA, 20 bp, synthetic construct
  misc_feature 1..20: note = Synthetic polynucleotide
  source 1..20: mol_type = other DNA
tattagatctgatggccgcg

SEQ ID NO: 31 - DNA, 20 bp, synthetic construct
  misc_feature 1..20: note = Synthetic polynucleotide
  source 1..20: mol_type = other DNA
tccatcactaggggttcctg

SEQ ID NO: 32 - DNA, 4013 bp, synthetic construct
  misc_feature 1..4013: note = Synthetic polynucleotide
  source 1..4013: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 33 - DNA, 4013 bp, synthetic construct; identical to SEQ ID NO: 32
  misc_feature 1..4013: note = Synthetic polynucleotide
  source 1..4013: mol_type = other DNA

SEQ ID NO: 34 - DNA, 4162 bp, synthetic construct
  misc_feature 1..4162: note = Synthetic polynucleotide
  source 1..4162: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtctttagaaataagtggtagtcaagtgaagccacagatgtgactaccacttatttctaaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 35 - DNA, 4162 bp, synthetic construct; identical to SEQ ID NO: 34
  misc_feature 1..4162: note = Synthetic polynucleotide
  source 1..4162: mol_type = other DNA

SEQ ID NO: 36 - DNA, 3977 bp, synthetic construct
  misc_feature 1..3977: note = Synthetic polynucleotide
  source 1..3977: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaactgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggcccacatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 37 - DNA, 4013 bp, synthetic construct; identical to SEQ ID NO: 32
  misc_feature 1..4013: note = Synthetic polynucleotide
  source 1..4013: mol_type = other DNA

SEQ ID NO: 38 - DNA, 4606 bp, synthetic construct
  misc_feature 1..4606: note = Synthetic polynucleotide
  source 1..4606: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccaccgcagccctaggaatgcatctagacaattgtactaaccttcttctctttcctctcctgacagtccggaaagccaccatgtacgccctgttcctgctggccagcctgctgggcgccgccctggccggccccgtgctgggcctgaaggagtgcacccgcggcagcgccgtgtggtgccagaacgtgaagaccgccagcgactgcggcgccgtgaagcactgcctgcagaccgtgtggaacaagcccaccgtgaagagcctgccctgcgacatctgcaaggacgtggtgaccgccgccggcgacatgctgaaggacaacgccaccgaggaggagatcctggtgtacctggagaagacctgcgactggctgcccaagcccaacatgagcgccagctgcaaggagatcgtggacagctacctgcccgtgatcctggacatcatcaagggcgagatgagccgccccggcgaggtgtgcagcgccctgaacctgtgcgagagcctgcagaagcacctggccgagctgaaccaccagaagcagctggagagcaacaagatccccgagctggacatgaccgaggtggtggcccccttcatggccaacatccccctgctgctgtacccccaggacggcccccgcagcaagccccagcccaaggacaacggcgacgtgtgccaggactgcatccagatggtgaccgacatccagaccgccgtgcgcaccaacagcaccttcgtgcaggccctggtggagcacgtgaaggaggagtgcgaccgcctgggccccggcatggccgacatctgcaagaactacatcagccagtacagcgagatcgccatccagatgatgatgcacatgcagcccaaggagatctgcgccctggtgggcttctgcgacgaggtgaaggagatgcccatgcagaccctggtgcccgccaaggtggccagcaagaacgtgatccccgccctggagctggtggagcccatcaagaagcacgaggtgcccgccaagagcgacgtgtactgcgaggtgtgcgagttcctggtgaaggaggtgaccaagctgatcgacaacaacaagaccgagaaggagatcctggacgccttcgacaagatgtgcagcaagctgcccaagagcctgagcgaggagtgccaggaggtggtggacacctacggcagcagcatcctgagcatcctgctggaggaggtgagccccgagctggtgtgcagcatgctgcacctgtgcagcggcacccgcctgcccgccctgaccgtgcacgtgacccagcccaaggacggcggcttctgcgaggtgtgcaagaagctggtgggctacctggaccgcaacctggagaagaacagcaccaagcaggagatcctggccgccctggagaagggctgcagcttcctgcccgacccctaccagaagcagtgcgaccagttcgtggccgagtacgagcccgtgctgatcgagatcctggtggaggtgatggaccccagcttcgtgtgcctgaagatcggcgcctgccccagcgcccacaagcccctgctgggcaccgagaagtgcatctggggccccagctactggtgccagaacaccgagaccgccgcccagtgcaacgccgtggagcactgcaagcgccacgtgtggaacagaagaaagagaggaagtggagagggcagaggaagtcttctgacatgcggagacgtggaagagaatcccggccctatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgtacactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 39 - DNA, 10699 bp, synthetic construct
  misc_feature 1..10699: note = PR001A vector first strand sequence
  source 1..10699: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 40 - DNA, 10699 bp, synthetic construct
  misc_feature 1..10699: note = PR001A vector second strand sequence
  source 1..10699: mol_type = other DNA
gactgccggacgtcgacttggcgcgccctcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggttttctctacactgaagtcttgatggcatgcgctttagtctcctcactcatgaggactgcatatctaagagctctgtgacctcacttgtgcctggtgtccagggtaaacaaatcttcttgcctttgaaaattagaggatgcagactggtctcctctactgcactggactgactaacctgactggagctcttgttcacactgagaagaagctggtggactcttctttggctctgaaaggcagatttattttatctcatggagttaggctagagcatcttgtcagagggaatgatgtttgaagtccatgaggcttctaatgattagggctggtatgcagcaaacaaccatgactgcatttgtctctgatgcagattcctaacttctgatatgttcaaaactctgcttcctgattcagttgctctctttaacaaatgcagtgattcttttgataatttgaggggaaaaaatgtctctgtgttgtgtttcctccttgctggtgatccacagtaaggggttgaaaataatctctgacattttttagttaaagccttcctttcattcttcatggctactgcagcacaaagctctttcttgttttgcagaaggagtgagagcaaggtgttgggtgagttctcagtgccccagccccagcctgaagaggagactgaatgcctgccctctggctgtggctcagagtgaaaggaaaacacatacagcaggagtctgtgtagggattgggctccagccatcagagcggggggagcatcatgaaatgtttgtaagctctccaccatccagaatgactttgcagctctgagcagctgcacccatgactagtccttcacagtatgctgatgtgagatattctgttctttgaagatgggtagtgaccagaatcaggaggatgtgagaaatgtggagcatttgagagtggaactcctgcagctgttgaagctagaacacaacagacactgcagcttactactcctgagcctggccaggctggtagttttgtttttcttggccaagctctctgggaatagttctgcctctggggtcagctttccatgcaggggctgagatgaacacataaagcaggattcagcttggaggcttctgagagggatggagataaaagatggtgtggtagatggtaagaggtggtatggtagatggcagagtatggcagaagatgtagacaggatgtcagaaaatgagatggtagttgcaagagtaggctgtagacagtgggagatggtgagatgagagatgcagaagatggtcagagagcaggatgttgggagaggctaggaggtgagacaccagggttggagtgtggactgcaggctttgttaggctggagaggtgagtagctgtggagagaggatgacctgcacttgtctgaatctaacagaacctaaattagaaactttctcttgcagttctctctctggctcaggctggcttgtcttggggactgtctgtcactctggaacagggtggttagagctgtggctttcatggtacttctcttcactgggctctgatagcctggggctgctggtactatctgcttgcacttgctttgaggaggccaagaacatcctgtgtatttccagactctttcttgttctgtttgtcctctcaggctattccttgtgctcctgatgtctctgtctttacttccaaaactatttattaaaaaatctaagattcattcatgtttaaggggaattatgctaattctttcatacctaatgggataaaactaactgtgaaattacagaccatcttgtgtaaaatcttaatctcagcagtcacatttgctgttgaagtgagtatgaacaaacactgatatgtggagggctctggtgcaaaggtttgtaggctctgcctgtgttgcatagagcagctcatctgaagtggattagcactgcttctgagcatggaaggtcaggctgaagtcatgatgcctaattagggctggagaggacatcacagaaacagtggctcttgggtttgtcctgggtagggcagaggtaaggttgaggagagctagaaaactcaggaaaagaaaaggctcaggagaaagaagacagagagttccagtgagcagagctgcctctgctgctaggcttgtggtacacaggttaaagtttggccttgtggcattcaacagcctttgcatcctactgaccacactagccaggcagttgaggctcagggctggctttcttgcacttggagaacctttcctaccttcaaatgcttgaaccagaataccagagaaaaaatgcaactctgaaagcacaggcattctgtgaatagccatccagatttctgcaggctaaggaaaacagagatggaggtctatgagggaaagaagagaagtcctcatgagggctgataccagacagttgagtaggacactgagcacactcaccaccagatgctctgtctaagctgtagtttactaggaggttgtttattctctgagtcagatttgcttctgttttaagaaccctcacagcctctctttctatctattctgatgaaggttgagcctgaagaagctggagcagaaagggtcttctgcctcatgttctgctgataaactttgagaatcctgcctaagtctgacctagattttggctccaaaagccagggcttcttatctctgtcaagaccaaacctggatgcagtcagaggttgggcacagcctttgctccaaggctcctgggcacagtgcccacctcccatttgagcttgcagcaaggctattatgagaaactttcctcctcttccttcaagtctccacctattagctctgcaagagtgcagaaggctgcacagcactgcagatggagaaggtggcaggggcagcaggtggcaggggctccagggaactgtggtcagagttgattcagagcaggctaagagtggagtgactgaagacagaagctggaggcacatcaaccattacaagacagcttgacttttgctagaacccttggcttcaggtgatgagaataatggtagacactggttgtttggcttgatagcttaaaaaaggacaaccattataatggaagcagtttctctgagaaacaggatgttggcaagggcttagtgctcctgactgctgatgctggagtctggcttccctgaccccagtagcagtgggacttagaagcatctcaggctccaggttctcacagctgactcctccttcctgaactgtaagaaacagaaatccacttgctgctctaaagtggggtcactttaatggaaggaacagccaacatatttggagaggggctggtgtggggcttcttactgagagtgggcagccctgagcactggacaaactcaactgtgggcaacctgggtgggaagcagctgtggagatggggaagccatgctaatcagtgacatcatctattctaagtttcctacctctgaatgaatagttctgctggcaaaaatctgcttttttaagttgatacaaatgtgtcctgtcaaggaagtagagctccttctcaatccagcacatcagtaccataacttgttcctgtgcatttggttaaagatggtgataacatcatttatcagtaagtgcagccaggcatgatgcactatctccttgcatttagagcctaattttggccaaagaagtcttcatagaaaaggacttcccactcaaagtagtcctgagtagttctactgattttattattagtagagagggtttcatcatgttggccaggctggtctgcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagaccatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttatcctgcaggaattcctcgagaccgtacgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagggaattcagtcaatatgttcaccccaaaaaagctgtttgttatcgtggatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgcggcctcgagggtttaaacttaattaacaattgtcactgtctacgccacaggtaggtgtggatggagtagccagggctgattgtttccaggaatcccacggcgggatccttgatggtcaggggcacatctttgctgctgcggttcaggaccaccaccacagcagatccatcagggtgcatcagagccacggcgtccagatcgttcttctgggaagccaccagtccaacgcgctgagagccctcggggatgaacttgctgaagtgtcccaggtggtagaacatgggctgcttgtagaaggtgtccttggtgatgtccacgatgatggggctgtccacgaagtttcggacccagttagggccgccttcaggattcagggccagattccagtcggtccagccgacgacgtggtacagcaggttggtgatgatgctgtggctgtactgcatgcctctatcccagctgccgagccgcacgctctgttcccaaaacttgctgcccacacaggcttcgctggcgaacagcatggtgttggggaacagtctgtgtgtctctcccagtgtggccttggcaggggccagaaagtccagataccagtgcacggcgattccgtgcacgtatttggcggcctcaggatctgtcagcaccactttagcccagtggggcagaagcagtctctggtcgtccagcatcagcagccgcacattatggtgggtgctattggccagtgtgggtcccagatcacgggcgataaagtcccgctggtgctcgggtgtaaagcccaggcactgaaaggggtagccgctcagcagtccagcagaaggttcgttctcggctgtcacggcccaaaactgcagcttgtgctcggcataggcgtccaggaacttcacgaagtatctggcccaggtctggtggtagatgtcgccaggttggcctttcaggctgcccttgccattcacggcgccatttgttttcagccaggtgggagatgtccatggagaggccagcagtgacacgggtctttgtgccagctgcagggctctgtggatcagagggatcttcagcttggtgtcctcttcaggcaggctgaagttgtgcagctggaaatcgtcgggtgtgtcggcgtaggtgtaggtcctgatgctgaagtcgcagctggccatgggcactctgatgatgttgtagccgatgccttcctcgctgaagtagctcttgagcagcaggttctgagctggtggagacagagccaggatattcagagcggcggcatctgtcatggctccgccgaagcctttcactttctggaatttctgctcaggctgcagtgtcagcagcaggccagtgcctgtgtgattggcctggatgggtcccatgctcagttccatccgtctgccggatctggtgctctcgtatctgctgaaggtgcccagagcaggaaaggtaggagggtcgaagctgtcgcagtaggtggcattgcacacgcacacgacgctgctgtagccgaagctcttggggatgcaaggtctagcgccagaagcccaagacacggcctgaagcagcagcagtcctgtcagagatccggccatgattgacacccggctcagaggcttggggcattcctctctgctggggctgctgaattccatggtggctcaaccggtgatatcttcgaacggatcccacagtcgtggaagactttcccttcggatcttcgaggaattctttgccaaaatgatgagacagcacaataaccagcacgttgcccaggagctgtaggaaaaagaagaaggcatgaacatggttagcagaggctctagctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattcagatccaagcttcaccatgggagacgtcaccggttctagaacctagggagctctggtacccactagtagtcgacgaacgcgtaacctcccgcttcaaaatggagaccctgcgtgctcactcgggcttaaatacccagagctagcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 10960 bp, synthetic construct; identical to SEQ ID NO: 13
  misc_feature 1..10960: note = PR004B vector first strand sequence
  source 1..10960: mol_type = other DNA

SEQ ID NO: 42 - DNA, 10960 bp, synthetic construct
  misc_feature 1..10960: note = PR004B vector second strand sequence
  source 1..10960: mol_type = other DNA
gactgccggacgtcgacttggcgcgccctcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggttttctctacactgaagtcttgatggcatgcgctttagtctcctcactcatgaggactgcatatctaagagctctgtgacctcacttgtgcctggtgtccagggtaaacaaatcttcttgcctttgaaaattagaggatgcagactggtctcctctactgcactggactgactaacctgactggagctcttgttcacactgagaagaagctggtggactcttctttggctctgaaaggcagatttattttatctcatggagttaggctagagcatcttgtcagagggaatgatgtttgaagtccatgaggcttctaatgattagggctggtatgcagcaaacaaccatgactgcatttgtctctgatgcagattcctaacttctgatatgttcaaaactctgcttcctgattcagttgctctctttaacaaatgcagtgattcttttgataatttgaggggaaaaaatgtctctgtgttgtgtttcctccttgctggtgatccacagtaaggggttgaaaataatctctgacattttttagttaaagccttcctttcattcttcatggctactgcagcacaaagctctttcttgttttgcagaaggagtgagagcaaggtgttgggtgagttctcagtgccccagccccagcctgaagaggagactgaatgcctgccctctggctgtggctcagagtgaaaggaaaacacatacagcaggagtctgtgtagggattgggctccagccatcagagcggggggagcatcatgaaatgtttgtaagctctccaccatccagaatgactttgcagctctgagcagctgcacccatgactagtccttcacagtatgctgatgtgagatattctgttctttgaagatgggtagtgaccagaatcaggaggatgtgagaaatgtggagcatttgagagtggaactcctgcagctgttgaagctagaacacaacagacactgcagcttactactcctgagcctggccaggctggtagttttgtttttcttggccaagctctctgggaatagttctgcctctggggtcagctttccatgcaggggctgagatgaacacataaagcaggattcagcttggaggcttctgagagggatggagataaaagatggtgtggtagatggtaagaggtggtatggtagatggcagagtatggcagaagatgtagacaggatgtcagaaaatgagatggtagttgcaagagtaggctgtagacagtgggagatggtgagatgagagatgcagaagatggtcagagagcaggatgttgggagaggctaggaggtgagacaccagggttggagtgtggactgcaggctttgttaggctggagaggtgagtagctgtggagagaggatgacctgcacttgtctgaatctaacagaacctaaattagaaactttctcttgcagttctctctctggctcaggctggcttgtcttggggactgtctgtcactctggaacagggtggttagagctgtggctttcatggtacttctcttcactgggctctgatagcctggggctgctggtactatctgcttgcacttgctttgaggaggccaagaacatcctgtgtatttccagactctttcttgttctgtttgtcctctcaggctattccttgtgctcctgatgtctctgtctttacttccaaaactatttattaaaaaatctaagattcattcatgtttaaggggaattatgctaattctttcatacctaatgggataaaactaactgtgaaattacagaccatcttgtgtaaaatcttaatctcagcagtcacatttgctgttgaagtgagtatgaacaaacactgatatgtggagggctctggtgcaaaggtttgtaggctctgcctgtgttgcatagagcagctcatctgaagtggattagcactgcttctgagcatggaaggtcaggctgaagtcatgatgcctaattagggctggagaggacatcacagaaacagtggctcttgggtttgtcctgggtagggcagaggtaaggttgaggagagctagaaaactcaggaaaagaaaaggctcaggagaaagaagacagagagttccagtgagcagagctgcctctgctgctaggcttgtggtacacaggttaaagtttggccttgtggcattcaacagcctttgcatcctactgaccacactagccaggcagttgaggctcagggctggctttcttgcacttggagaacctttcctaccttcaaatgcttgaaccagaataccagagaaaaaatgcaactctgaaagcacaggcattctgtgaatagccatccagatttctgcaggctaaggaaaacagagatggaggtctatgagggaaagaagagaagtcctcatgagggctgataccagacagttgagtaggacactgagcacactcaccaccagatgctctgtctaagctgtagtttactaggaggttgtttattctctgagtcagatttgcttctgttttaagaaccctcacagcctctctttctatctattctgatgaaggttgagcctgaagaagctggagcagaaagggtcttctgcctcatgttctgctgataaactttgagaatcctgcctaagtctgacctagattttggctccaaaagccagggcttcttatctctgtcaagaccaaacctggatgcagtcagaggttgggcacagcctttgctccaaggctcctgggcacagtgcccacctcccatttgagcttgcagcaaggctattatgagaaactttcctcctcttccttcaagtctccacctattagctctgcaagagtgcagaaggctgcacagcactgcagatggagaaggtggcaggggcagcaggtggcaggggctccagggaactgtggtcagagttgattcagagcaggctaagagtggagtgactgaagacagaagctggaggcacatcaaccattacaagacagcttgacttttgctagaacccttggcttcaggtgatgagaataatggtagacactggttgtttggcttgatagcttaaaaaaggacaaccattataatggaagcagtttctctgagaaacaggatgttggcaagggcttagtgctcctgactgctgatgctggagtctggcttccctgaccccagtagcagtgggacttagaagcatctcaggctccaggttctcacagctgactcctccttcctgaactgtaagaaacagaaatccacttgctgctctaaagtggggtcactttaatggaaggaacagccaacatatttggagaggggctggtgtggggcttcttactgagagtgggcagccctgagcactggacaaactcaactgtgggcaacctgggtgggaagcagctgtggagatggggaagccatgctaatcagtgacatcatctattctaagtttcctacctctgaatgaatagttctgctggcaaaaatctgcttttttaagttgatacaaatgtgtcctgtcaaggaagtagagctccttctcaatccagcacatcagtaccataacttgttcctgtgcatttggttaaagatggtgataacatcatttatcagtaagtgcagccaggcatgatgcactatctccttgcatttagagcctaattttggccaaagaagtcttcatagaaaaggacttcccactcaaagtagtcctgagtagttctactgattttattattagtagagagggtttcatcatgttggccaggctggtctgcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagaccatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttatcctgcaggaattcctcgagaccgtacgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagggaattcagtcaatatgttcaccccaaaaaagctgtttgttatcgtggatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgcggcctcgagggtttaaacttaattaacaattgtcactgtctacgccacaggtaggtgtggatggagtagccagggctgattgtttccaggaatcccacggcgggatccttgatggtcaggggcacatctttgctgctgcggttcaggaccaccaccacagcagatccatcagggtgcatcagagccacggcgtccagatcgttcttctgggaagccaccagtccaacgcgctgagagccctcggggatgaacttgctgaagtgtcccaggtggtagaacatgggctgcttgtagaaggtgtccttggtgatgtccacgatgatggggctgtccacgaagtttcggacccagttagggccgccttcaggattcagggccagattccagtcggtccagccgacgacgtggtacagcaggttggtgatgatgctgtggctgtactgcatgcctctatcccagctgccgagccgcacgctctgttcccaaaacttgctgcccacacaggcttcgctggcgaacagcatggtgttggggaacagtctgtgtgtctctcccagtgtggccttggcaggggccagaaagtccagataccagtgcacggcgattccgtgcacgtatttggcggcctcaggatctgtcagcaccactttagcccagtggggcagaagcagtctctggtcgtccagcatcagcagccgcacattatggtgggtgctattggccagtgtgggtcccagatcacgggcgataaagtcccgctggtgctcgggtgtaaagcccaggcactgaaaggggtagccgctcagcagtccagcagaaggttcgttctcggctgtcacggcccaaaactgcagcttgtgctcggcataggcgtccaggaacttcacgaagtatctggcccaggtctggtggtagatgtcgccaggttggcctttcaggctgcccttgccattcacggcgccatttgttttcagccaggtgggagatgtccatggagaggccagcagtgacacgggtctttgtgccagctgcagggctctgtggatcagagggatcttcagcttggtgtcctcttcaggcaggctgaagttgtgcagctggaaatcgtcgggtgtgtcggcgtaggtgtaggtcctgatgctgaagtcgcagctggccatgggcactctgatgatgttgtagccgatgccttcctcgctgaagtagctcttgagcagcaggttctgagctggtggagacagagccaggatattcagagcggcggcatctgtcatggctccgccgaagcctttcactttctggaatttctgctcaggctgcagtgtcagcagcaggccagtgcctgtgtgattggcctggatgggtcccatgctcagttccatccgtctgccggatctggtgctctcgtatctgctgaaggtgcccagagcaggaaaggtaggagggtcgaagctgtcgcagtaggtggcattgcacacgcacacgacgctgctgtagccgaagctcttggggatgcaaggtctagcgccagaagcccaagacacggcctgaagcagcagcagtcctgtcagagatccggccatgattgacacccggctcagaggcttggggcattcctctctgctggggctgctgaattccatggtggctcaaccggtgatatcttcgaacggatcccacagtcgtggaagactttcccttcggatcttcgaggaattctttgccaaaatgatgagacagcacaataaccagcacgttgcccaggagctgtaggaaaaagaagaaggcatgaacatggttagcagaggctctagctcccggagcccctcaaggctttcacgcagccacagaaaggtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccaggacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagggtaccgaattcagatccaagcttcaccatgggagacgtcaccggttctagaacctagggagctctggtacccactagtagtcgacgaacgcgtaacctcccgcttcaaaatggagaccctgcgtgctcactcgggcttaaatacccagagctagcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 43 - DNA, 10960 bp, synthetic construct
  misc_feature 1..10960: note = PR004E vector first strand sequence
  source 1..10960: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacctttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 44 - DNA, 10960 bp, synthetic construct
  misc_feature 1..10960: note = PR004E vector second strand sequence
  source 1..10960: mol_type = other DNA
gactgccggacgtcgacttggcgcgccctcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggttttctctacactgaagtcttgatggcatgcgctttagtctcctcactcatgaggactgcatatctaagagctctgtgacctcacttgtgcctggtgtccagggtaaacaaatcttcttgcctttgaaaattagaggatgcagactggtctcctctactgcactggactgactaacctgactggagctcttgttcacactgagaagaagctggtggactcttctttggctctgaaaggcagatttattttatctcatggagttaggctagagcatcttgtcagagggaatgatgtttgaagtccatgaggcttctaatgattagggctggtatgcagcaaacaaccatgactgcatttgtctctgatgcagattcctaacttctgatatgttcaaaactctgcttcctgattcagttgctctctttaacaaatgcagtgattcttttgataatttgaggggaaaaaatgtctctgtgttgtgtttcctccttgctggtgatccacagtaaggggttgaaaataatctctgacattttttagttaaagccttcctttcattcttcatggctactgcagcacaaagctctttcttgttttgcagaaggagtgagagcaaggtgttgggtgagttctcagtgccccagccccagcctgaagaggagactgaatgcctgccctctggctgtggctcagagtgaaaggaaaacacatacagcaggagtctgtgtagggattgggctccagccatcagagcggggggagcatcatgaaatgtttgtaagctctccaccatccagaatgactttgcagctctgagcagctgcacccatgactagtccttcacagtatgctgatgtgagatattctgttctttgaagatgggtagtgaccagaatcaggaggatgtgagaaatgtggagcatttgagagtggaactcctgcagctgttgaagctagaacacaacagacactgcagcttactactcctgagcctggccaggctggtagttttgtttttcttggccaagctctctgggaatagttctgcctctggggtcagctttccatgcaggggctgagatgaacacataaagcaggattcagcttggaggcttctgagagggatggagataaaagatggtgtggtagatggtaagaggtggtatggtagatggcagagtatggcagaagatgtagacaggatgtcagaaaatgagatggtagttgcaagagtaggctgtagacagtgggagatggtgagatgagagatgcagaagatggtcagagagcaggatgttgggagaggctaggaggtgagacaccagggttggagtgtggactgcaggctttgttaggctggagaggtgagtagctgtggagagaggatgacctgcacttgtctgaatctaacagaacctaaattagaaactttctcttgcagttctctctctggctcaggctggcttgtcttggggactgtctgtcactctggaacagggtggttagagctgtggctttcatggtacttctcttcactgggctctgatagcctggggctgctggtactatctgcttgcacttgctttgaggaggccaagaacatcctgtgtatttccagactctttcttgttctgtttgtcctctcaggctattccttgtgctcctgatgtctctgtctttacttccaaaactatttattaaaaaatctaagattcattcatgtttaaggggaattatgctaattctttcatacctaatgggataaaactaactgtgaaattacagaccatcttgtgtaaaatcttaatctcagcagtcacatttgctgttgaagtgagtatgaacaaacactgatatgtggagggctctggtgcaaaggtttgtaggctctgcctgtgttgcatagagcagctcatctgaagtggattagcactgcttctgagcatggaaggtcaggctgaagtcatgatgcctaattagggctggagaggacatcacagaaacagtggctcttgggtttgtcctgggtagggcagaggtaaggttgaggagagctagaaaactcaggaaaagaaaaggctcaggagaaagaagacagagagttccagtgagcagagctgcctctgctgctaggcttgtggtacacaggttaaagtttggccttgtggcattcaacagcctttgcatcctactgaccacactagccaggcagttgaggctcagggctggctttcttgcacttggagaacctttcctaccttcaaatgcttgaaccagaataccagagaaaaaatgcaactctgaaagcacaggcattctgtgaatagccatccagatttctgcaggctaaggaaaacagagatggaggtctatgagggaaagaagagaagtcctcatgagggctgataccagacagttgagtaggacactgagcacactcaccaccagatgctctgtctaagctgtagtttactaggaggttgtttattctctgagtcagatttgcttctgttttaagaaccctcacagcctctctttctatctattctgatgaaggttgagcctgaagaagctggagcagaaagggtcttctgcctcatgttctgctgataaactttgagaatcctgcctaagtctgacctagattttggctccaaaagccagggcttcttatctctgtcaagaccaaacctggatgcagtcagaggttgggcacagcctttgctccaaggctcctgggcacagtgcccacctcccatttgagcttgcagcaaggctattatgagaaactttcctcctcttccttcaagtctccacctattagctctgcaagagtgcagaaggctgcacagcactgcagatggagaaggtggcaggggcagcaggtggcaggggctccagggaactgtggtcagagttgattcagagcaggctaagagtggagtgactgaagacagaagctggaggcacatcaaccattacaagacagcttgacttttgctagaacccttggcttcaggtgatgagaataatggtagacactggttgtttggcttgatagcttaaaaaaggacaaccattataatggaagcagtttctctgagaaacaggatgttggcaagggcttagtgctcctgactgctgatgctggagtctggcttccctgaccccagtagcagtgggacttagaagcatctcaggctccaggttctcacagctgactcctccttcctgaactgtaagaaacagaaatccacttgctgctctaaagtggggtcactttaatggaaggaacagccaacatatttggagaggggctggtgtggggcttcttactgagagtgggcagccctgagcactggacaaactcaactgtgggcaacctgggtgggaagcagctgtggagatggggaagccatgctaatcagtgacatcatctattctaagtttcctacctctgaatgaatagttctgctggcaaaaatctgcttttttaagttgatacaaatgtgtcctgtcaaggaagtagagctccttctcaatccagcacatcagtaccataacttgttcctgtgcatttggttaaagatggtgataacatcatttatcagtaagtgcagccaggcatgatgcactatctccttgcatttagagcctaattttggccaaagaagtcttcatagaaaaggacttcccactcaaagtagtcctgagtagttctactgattttattattagtagagagggtttcatcatgttggccaggctggtctgcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagaccatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttatcctgcaggaattcctcgagaccgtacgagcggccgcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctgcagggaattcagtcaatatgttcaccccaaaaaagctgtttgttatcgtggatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgcggcctcgagggtttaaacttaattaacaattgtcactgtctacgccacaggtaggtgtggatggagtagccagggctgattgtttccaggaatcccacggcgggatccttgatggtcaggggcacatctttgctgctgcggttcaggaccaccaccacagcagatccatcagggtgcatcagagccacggcgtccagatcgttcttctgggaagccaccagtccaacgcgctgagagccctcggggatgaacttgctgaagtgtcccaggtggtagaacatgggctgcttgtagaaggtgtccttggtgatgtccacgatgatggggctgtccacgaagtttcggacccagttagggccgccttcaggattcagggccagattccagtcggtccagccgacgacgtggtacagcaggttggtgatgatgctgtggctgtactgcatgcctctatcccagctgccgagccgcacgctctgttcccaaaacttgctgcccacacaggcttcgctggcgaacagcatggtgttggggaacagtctgtgtgtctctcccagtgtggccttggcaggggccagaaagtccagataccagtgcacggcgattccgtgcacgtatttggcggcctcaggatctgtcagcaccactttagcccagtggggcagaagcagtctctggtcgtccagcatcagcagccgcacattatggtgggtgctattggccagtgtgggtcccagatcacgggcgataaagtcccgctggtgctcgggtgtaaagcccaggcactgaaaggggtagccgctcagcagtccagcagaaggttcgttctcggctgtcacggcccaaaactgcagcttgtgctcggcataggcgtccaggaacttcacgaagtatctggcccaggtctggtggtagatgtcgccaggttggcctttcaggctgcccttgccattcacggcgccatttgttttcagccaggtgggagatgtccatggagaggccagcagtgacacgggtctttgtgccagctgcagggctctgtggatcagagggatcttcagcttggtgtcctcttcaggcaggctgaagttgtgcagctggaaatcgtcgggtgtgtcggcgtaggtgtaggtcctgatgctgaagtcgcagctggccatgggcactctgatgatgttgtagccgatgccttcctcgctgaagtagctcttgagcagcaggttctgagctggtggagacagagccaggatattcagagcggcggcatctgtcatggctccgccgaagcctttcactttctggaatttctgctcaggctgcagtgtcagcagcaggccagtgcctgtgtgattggcctggatgggtcccatgctcagttccatccgtctgccggatctggtgctctcgtatctgctgaaggtgcccagagcaggaaaggtaggagggtcgaagctgtcgcagtaggtggcattgcacacgcacacgacgctgctgtagccgaagctcttggggatgcaaggtctagcgccagaagcccaagacacggcctgaagcagcagcagtcctgtcagagatccggccatgattgacacccggctcagaggcttggggcattcctctctgctggggctgctgaattccatggtggctcaaccggtgatatcttcgaacggatcccacagtcgtggaagactttcccttcggatcttcgaggaattctttgccaaaatgatgagacagcacaataaccagcacgttgcccaggagctgtaggaaaaagaagaaggcatgaacatggttagcagaggctctagctcccggagcccctcaaggctttcacgcagccacagaaaggtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccaggacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagggtaccgaattcagatccaagcttcaccatgggagacgtcaccggttctagaacctagggagctctggtacccactagtagtcgacgaacgcgtaacctcccgcttcaaaatggagaccctgcgtgctcactcgggcttaaatacccagagctagcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 45 - DNA, 9348 bp, synthetic construct
  misc_feature 1..9348: note = PR014A vector first strand sequence
  source 1..9348: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgtcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacctttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccacccaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 46 - DNA, 9348 bp, synthetic construct
  misc_feature 1..9348: note = PR014A vector second strand sequence
  source 1..9348: mol_type = other DNA
gactgccggacgtcgacttggcgcgccctcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggttttctctacactgaagtcttgatggcatgcgctttagtctcctcactcatgaggactgcatatctaagagctctgtgacctcacttgtgcctggtgtccagggtaaacaaatcttcttgcctttgaaaattagaggatgcagactggtctcctctactgcactggactgactaacctgactggagctcttgttcacactgagaagaagctggtggactcttctttggctctgaaaggcagatttattttatctcatggagttaggctagagcatcttgtcagagggaatgatgtttgaagtccatgaggcttctaatgattagggctggtatgcagcaaacaaccatgactgcatttgtctctgatgcagattcctaacttctgatatgttcaaaactctgcttcctgattcagttgctctctttaacaaatgcagtgattcttttgataatttgaggggaaaaaatgtctctgtgttgtgtttcctccttgctggtgatccacagtaaggggttgaaaataatctctgacattttttagttaaagccttcctttcattcttcatggctactgcagcacaaagctctttcttgttttgcagaaggagtgagagcaaggtgttgggtgagttctcagtgccccagccccagcctgaagaggagactgaatgcctgccctctggctgtggctcagagtgaaaggaaaacacatacagcaggagtctgtgtagggattgggctccagccatcagagcggggggagcatcatgaaatgtttgtaagctctccaccatccagaatgactttgcagctctgagcagctgcacccatgactagtccttcacagtatgctgatgtgagatattctgttctttgaagatgggtagtgaccagaatcaggaggatgtgagaaatgtggagcatttgagagtggaactcctgcagctgttgaagctagaacacaacagacactgcagcttactactcctgagcctggccaggctggtagttttgtttttcttggccaagctctctgggaatagttctgcctctggggtcagctttccatgcaggggctgagatgaacacataaagcaggattcagcttggaggcttctgagagggatggagataaaagatggtgtggtagatggtaagaggtggtatggtagatggcagagtatggcagaagatgtagacaggatgtcagaaaatgagatggtagttgcaagagtaggctgtagacagtgggagatggtgagatgagagatgcagaagatggtcagagagcaggatgttgggagaggctaggaggtgagacaccagggttggagtgtggactgcaggctttgttaggctggagaggtgagtagctgtggagagaggatgacctgcacttgtctgaatctaacagaacctaaattagaaactttctcttgcagttctctctctggctcaggctggcttgtcttggggactgtctgtcactctggaacagggtggttagagctgtggctttcatggtacttctcttcactgggctctgatagcctggggctgctggtactatctgcttgcacttgctttgaggaggccaagaacatcctgtgtatttccagactctttcttgttctgtttgtcctctcaggctattccttgtgctcctgatgtctctgtctttacttccaaaactatttattaaaaaatctaagattcattcatgtttaaggggaattatgctaattctttcatacctaatgggataaaactaactgtgaaattacagaccatcttgtgtaaaatcttaatctcagcagtcacatttgctgttgaagtgagtatgaacaaacactgatatgtggagggctctggtgcaaaggtttgtaggctctgcctgtgttgcatagagcagctcatctgaagtggattagcactgcttctgagcatggaaggtcaggctgaagtcatgatgcctaattagggctggagaggacatcacagaaacagtggctcttgggtttgtcctgggtagggcagaggtaaggttgaggagagctagaaaactcaggaaaagaaaaggctcaggagaaagaagacagagagttccagtgagcagagctgcctctgctgctaggcttgtggtacacaggttaaagtttggccttgtggcattcaacagcctttgcatcctactgaccacactagccaggcagttgaggctcagggctggctttcttgcacttggagaacctttcctaccttcaaatgcttgaaccagaataccagagaaaaaatgcaactctgaaagcacaggcattctgtgaatagccatccagatttctgcaggctaaggaaaacagagatggaggtctatgagggaaagaagagaagtcctcatgagggctgataccagacagttgagtaggacactgagcacactcaccaccagatgctctgtctaagctgtagtttactaggaggttgtttattctctgagtcagatttgcttctgttttaagaaccctcacagcctctctttctatctattctgatgaaggttgagcctgaagaagctggagcagaaagggtcttctgcctcatgttctgctgataaactttgagaatcctgcctaagtctgacctagattttggctccaaaagccagggcttcttatctctgtcaagaccaaacctggatgcagtcagaggttgggcacagcctttgctccaaggctcctgggcacagtgcccacctcccatttgagcttgcagcaaggctattatgagaaactttcctcctcttccttcaagtctccacctattagctctgcaagagtgcagaaggctgcacagcactgcagatggagaaggtggcaggggcagcaggtggcaggggctccagggaactgtggtcagagttgattcagagcaggctaagagtggagtgactgaagacagaagctggaggcacatcaaccattacaagacagcttgacttttgctagaacccttggcttcaggtgatgagaataatggtagacactggttgtttggcttgatagcttaaaaaaggacaaccattataatggaagcagtttctctgagaaacaggatgttggcaagggcttagtgctcctgactgctgatgctggagtctggcttccctgaccccagtagcagtgggacttagaagcatctcaggctccaggttctcacagctgactcctccttcctgaactgtaagaaacagaaatccacttgctgctctaaagtggggtcactttaatggaaggaacagccaacatatttggagaggggctggtgtggggcttcttactgagagtgggcagccctgagcactggacaaactcaactgtgggcaacctgggtgggaagcagctgtggagatggggaagccatgctaatcagtgacatcatctattctaagtttcctacctctgaatgaatagttctgctggcaaaaatctgcttttttaagttgatacaaatgtgtcctgtcaaggaagtagagctccttctcaatccagcacatcagtaccataacttgttcctgtgcatttggttaaagatggtgataacatcatttatcagtaagtgcagccaggcatgatgcactatctccttgcatttagagcctaattttggccaaagaagtcttcatagaaaaggacttcccactcaaagtagtcctgagtagttctactgattttattattagtagagagggtttcatcatgttggccaggctggtctgcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagaccatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttatcctgcaggaattcctcgagaccgtacgagcggccgcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctgcagggaattcagtcaatatgttcaccccaaaaaagctgtttgttatcgtggatctctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgcggcctcgagggtttaaacttaattaacaattgggtggctcaaccggtgatatcttcgaacggatcccacagtcgtggaagactttcccttcggatcttcgaggaattctttgccaaaatgatgagacagcacaataaccagcacgttgcccaggagctgtaggaaaaagaagaaggcatgaacatggttagcagaggctctagctcccggagcccctcaaggctttcacgcagccacagaaaggtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccaggacaagccgtcattaaaccaagcgctaattacagcgctgaggagaagggccgtcccgcccgctcacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattcagatccaagcttcaccatgggagacgtcaccggttctagaacctagggagctctggtacccactagtagtcgacgaacgcgtaacctcccgcttcaaaatggagaccctgcgtgctcactcgggcttaaatacccagagctagcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - DNA, 263 bp, synthetic construct
  misc_feature 1..263: note = First strand sequence encoding aSyn shRNA
  source 1..263: mol_type = other DNA
cctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacc

SEQ ID NO: 48 - DNA, 263 bp, synthetic construct
  misc_feature 1..263: note = Second strand sequence encoding aSyn shRNA
  source 1..263: mol_type = other DNA
ggtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagg

(19) United States
(12) Patent Application Publication
(10) Pub. No.: US 2024/0123003 A1
(43) Pub. Date: Apr. 18, 2024

(54) GENE THERAPIES FOR LYSOSOMAL DISORDERS

(71) Applicant: Prevail Therapeutics, Inc., New York, NY (US)

(72) Inventors: Asa ABELIOVICH, New York, NY (US); Laura HECKMAN, New York, NY (US); Herve RHINN, New York, NY (US); Franz HEFTI, New York, NY (US)

(21) Appl. No.: 18/392,002

(22) Filed: Dec. 21, 2023

Related U.S. Application Data
(62) Division of application No. 16/846,210, filed on Apr. 10, 2020, now Pat. No. 11,903,985.
(60) Provisional application No. 62/990,246, filed on Mar. 16, 2020.
(60) Provisional application No. 62/831,840, filed on Apr. 10, 2019.

(51) Int. Cl. A61K 35/761; A61K 9/00; A61K 38/16; A61P 25/28; A61P 43/00; C12N 15/86

(52) U.S. Cl. CPC A61K 35/761; A61K 9/0085; A61K 38/162; A61P 25/28; A61P 43/00; C12N 15/86; A61K 9/0019; C12Y 302/01045

(57) ABSTRACT

The disclosure relates to compositions and methods for treatment of diseases associated with aberrant lysosomal function, such as Parkinson's disease and Gaucher disease. The disclosure provides expression constructs comprising a transgene encoding beta-glucocerebrosidase, an inhibitory RNA targeting alpha-Synuclein, or a combination of the foregoing. The disclosure further provides methods of treating Gaucher disease, Parkinson's disease or other synucleinopathies by administering such expression constructs to a subject in need thereof.

### CROSS-REFERENCE TO RELATED APPLICATIONS

This application is a division of U.S. patent application Ser. No. 16/846,210, filed on Apr. 10, 2020, which claims priority to U.S. Provisional Patent Application No. 62/990,246, filed on Mar. 16, 2020 and U.S. Provisional Patent Application No. 62/831,840, filed on Apr. 10, 2019. The disclosure of each of these applications is incorporated herein by reference in its entirety.

### REFERENCE TO AN ELECTRONIC SEQUENCE LISTING

The contents of the electronic sequence listing (PRVL_009_03US_SeqList_ST26.xml; Size: 322,283 bytes; and Date of Creation: Dec. 12, 2023) are herein incorporated by reference in their entirety.

### BACKGROUND

Aberrant expression of proteins such as lysosomal acid β-glucocerebrosidase (Gcase) and α-Synuclein is involved in many central nervous system disorders. Gaucher disease is a rare inborn error of glycosphingolipid metabolism due to deficiency of Gcase. Patients suffer from non-CNS symptoms and findings including hepatosplenomegaly, bone marrow insufficiency leading to pancytopenia, lung disorders and fibrosis, and bone defects. In addition, a significant number of patients suffer from neurological manifestations, including defective saccadic eye movements and gaze, seizures, cognitive deficits, developmental delay, and movement disorders including Parkinson's disease.

Several therapeutics exist that address the peripheral disease and the principal clinical manifestations in hematopoietic bone marrow and viscera, including enzyme replacement therapies, chaperone-like small molecule drugs that bind to defective Gcase and improve stability, and substrate reduction therapy that block the production of substrates that accumulate in Gaucher disease, leading to symptoms and pathology. However, other aspects of Gaucher disease appear refractory to treatment.

In addition to Gaucher disease patients (who possess mutations in both chromosomal alleles of GBA1 gene), patients with mutations in only one allele of GBA1 are at highly increased risk of Parkinson's disease (PD). Elevated α-Synuclein levels also underlie synucleinopathies such as PD. The severity of PD symptoms—which include gait difficulty, a tremor at rest, rigidity, and often depression, sleep difficulties, and cognitive decline—correlate with the degree of enzyme activity reduction. Thus, Gaucher disease patients have the most severe course, whereas patients with a single mild mutation in GBA1 typically have a more benign course. Mutation carriers are also at high risk of other PD-related disorders, including Lewy Body Dementia, characterized by executive dysfunction, psychosis, and a PD-like movement disorder, and multi-system atrophy, with characteristic motor and cognitive impairments. No therapies exist that alter the inexorable course of these disorders and other synucleinopathies.

### FIELD

The disclosure relates to the field of gene therapy and methods of using same.

### SUMMARY

Provided herein is a method for treating a subject having Type 2 Gaucher disease or Type 3 Gaucher disease, the method comprising administering to the subject a recombinant adeno-associated virus (rAAV) comprising: (i) a rAAV vector comprising a nucleic acid comprising an expression construct comprising a promoter operably linked to a transgene insert encoding a glucocerebrosidase (Gcase) protein, wherein the transgene insert comprises the nucleotide sequence of SEQ ID NO: 15; and (ii) an AAV9 capsid protein. In some embodiments, the rAAV is administered to the subject at a dose ranging from about 5×10¹⁰ vg/g brain to about 5×10¹¹ vg/g brain. In some embodiments, the rAAV is administered to the subject at a dose of about 1.3×10¹¹ vg/g brain.

Provided herein is a method for treating a subject having Parkinson's disease with a glucocerebrosidase-1 (GBA1) mutation, the method comprising administering to the subject a recombinant adeno-associated virus (rAAV) comprising: (i) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and (ii) an AAV9 capsid protein. In some embodiments, the rAAV is administered to the subject at a dose ranging from about 5×10¹³ vg to about 5×10¹⁴ vg. In some embodiments, the rAAV is administered to the subject at a dose of about 1×10¹⁴ vg or about 2×10¹⁴ vg.

In some embodiments, the rAAV is administered via a suboccipital injection into the cisterna magna.

Provided herein is a method for treating a subject having Type 1 Gaucher disease, the method comprising administering to the subject a recombinant adeno-associated virus (rAAV) comprising: (i) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and (ii) an AAV9 capsid protein. In some embodiments, the rAAV is administered to the subject at a dose ranging from about 5×10¹³ vg to about 5×10¹⁴ vg. In some embodiments, the rAAV is administered intravenously.

Provided herein is a method for treating a subject having a synucleinopathy or parkinsonism, the method comprising administering to the subject a recombinant adeno-associated virus (rAAV) comprising: (i) a rAAV vector comprising a nucleic acid comprising an expression construct comprising a transgene comprising (a) a Gcase protein coding sequence comprising the nucleotide sequence of SEQ ID NO: 15; and (b) an inhibitory nucleic acid coding sequence comprising the nucleotide sequence of SEQ ID NO: 20; and (ii) an AAV9 capsid protein.

Provided herein is a method for treating a subject having a synucleinopathy or parkinsonism, the method comprising administering to the subject a recombinant adeno-associated virus (rAAV) comprising: (i) a rAAV vector comprising a nucleic acid comprising an expression construct comprising a transgene comprising an inhibitory nucleic acid coding sequence comprising the nucleotide sequence of SEQ ID NO: 20; and an AAV9 capsid protein.

In some embodiments, the synucleinopathy or parkinsonism is multiple system atrophy, Parkinson's disease, Parkinson's disease with GBA1 mutation, Lewy body disease, dementia with Lewy bodies, dementia with Lewy bodies with GBA1 mutation, progressive supranuclear palsy, or corticobasal syndrome.

In some embodiments, the promoter is a chicken beta actin (CBA) promoter. In some embodiments, the rAAV vector further comprises a cytomegalovirus (CMV) enhancer. In some embodiments, the rAAV vector further comprises a Woodchuck Hepatitis Virus Posttranscriptional Regulatory Element (WPRE). In some embodiments, the rAAV vector further comprises a Bovine Growth Hormone polyA signal tail. In some embodiments, the nucleic acid comprises two adeno-associated virus inverted terminal repeats (ITR) sequences flanking the expression construct. In some embodiments, each ITR sequence is a wild-type AAV2 ITR sequence. In some embodiments, the rAAV vector further comprises a TRY region between the 5′ ITR and the expression construct, wherein the TRY region comprises SEQ ID NO: 28.

Provided herein is a method for treating a subject having Type 2 Gaucher disease or Type 3 Gaucher disease, the method comprising administering to the subject a rAAV comprising:
(i) a rAAV vector comprising a nucleic acid comprising, in 5′ to 3′ order:
   (a) an AAV2 ITR;
   (b) a CMV enhancer;
   (c) a CBA promoter;
   (d) a transgene insert encoding a Gcase protein, wherein the transgene insert comprises the nucleotide sequence of SEQ ID NO: 15;
   (e) a WPRE;
   (f) a Bovine Growth Hormone polyA signal tail; and
   (g) an AAV2 ITR; and
(ii) an AAV9 capsid protein;
wherein the rAAV is administered to the subject at a dose ranging from about 5×10¹⁰ vg/g brain to about 5×10¹¹ vg/g brain.

Provided herein is a method for treating a subject having Parkinson's disease with a GBA1 mutation, the method comprising administering to the subject a rAAV comprising:
(i) a rAAV vector comprising a nucleic acid comprising, in 5′ to 3′ order:
   (a) an AAV2 ITR;
   (b) a CMV enhancer;
   (c) a CBA promoter;
   (d) a transgene insert encoding a Gcase protein, wherein the transgene insert comprises the nucleotide sequence of SEQ ID NO: 15;
   (e) a WPRE;
   (f) a Bovine Growth Hormone polyA signal tail; and
   (g) an AAV2 ITR; and
(ii) an AAV9 capsid protein;
wherein the rAAV is administered to the subject at a dose ranging from about 5×10¹³ vg to about 5×10¹⁴ vg.

In some embodiments, the rAAV is administered via a suboccipital injection into the cisterna magna. In some embodiments, the rAAV is administered in a formulation comprising about 20 mM Tris, pH 8.0, about 1 mM MgCl₂, about 200 mM NaCl, and about 0.001% w/v poloxamer 188.

Provided herein is a pharmaceutical composition comprising
(i) a rAAV comprising:
   (a) a rAAV vector comprising a nucleic acid comprising an expression construct comprising a promoter operably linked to a transgene insert a Gcase protein, wherein the transgene insert comprises the nucleotide sequence of SEQ ID NO: 15; and
   (b) an AAV9 capsid protein; and
(ii) about 20 mM Tris, pH 8.0,
(iii) about 1 mM MgCl₂,
(iv) about 200 mM NaCl, and
(v) about 0.001% w/v poloxamer 188.

Provided herein is a rAAV comprising:
(a) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and
(b) an AAV9 capsid protein, for use in a method of treating Type 1 Gaucher disease, Type 2 Gaucher disease, Type 3 Gaucher disease or Parkinson's disease with a GBA1 mutation in a subject.

Provided herein is a rAAV comprising:
(i) a rAAV vector comprising a nucleic acid comprising an expression construct comprising a promoter operably linked to a transgene insert comprising:
   (a) a Gcase protein coding sequence comprising the nucleotide sequence of SEQ ID NO: 15; and
   (b) an inhibitory nucleic acid coding sequence comprising the nucleotide sequence of SEQ ID NO: 20; and
(ii) an AAV9 capsid protein, for use in a method of treating a synucleinopathy or parkinsonism in a subject.

Provided herein is a rAAV comprising:
(i) a rAAV vector comprising a nucleic acid comprising an expression construct comprising a promoter operably linked to a transgene insert comprising an inhibitory nucleic acid coding sequence comprising the nucleotide sequence of SEQ ID NO: 20; and
(ii) an AAV9 capsid protein, for use in a method of treating a synucleinopathy or parkinsonism in a subject.

### BRIEF DESCRIPTION OF THE DRAWINGS

FIG. 1 is a schematic depicting one embodiment of a plasmid comprising an rAAV vector that includes an expression construct encoding Gcase (e.g., GBA1 or a portion thereof).

FIG. 2 is a schematic depicting one embodiment of a plasmid comprising an rAAV vector that includes an expression construct encoding Gcase (e.g., GBA1 or a portion thereof) and LIMP2 (SCARB2) or a portion thereof. The coding sequences of Gcase and LIMP2 are separated by an internal ribosomal entry site (IRES).

FIG. 3 is a schematic depicting one embodiment of a plasmid comprising an rAAV vector that includes an expression construct encoding Gcase (e.g., GBA1 or a portion thereof) and LIMP2 (SCARB2) or a portion thereof. Expression of the coding sequences of Gcase and LIMP2 are each driven by a separate promoter.

FIG. 4 is a schematic depicting one embodiment of a plasmid comprising an rAAV vector that includes an expression construct encoding Gcase (e.g., GBA1 or a portion thereof), LIMP2 (SCARB2) or a portion thereof, and an interfering RNA for α-Syn.

FIG. 5 is a schematic depicting one embodiment of a plasmid comprising an rAAV vector that includes an expression construct encoding Gcase (e.g., GBA1 or a portion thereof), Prosaposin (e.g., PSAP or a portion thereof), and an interfering RNA for α-Syn.

FIG. 6 is a schematic depicting one embodiment of a plasmid comprising an rAAV vector that includes an expression construct encoding Gcase (e.g., GBA1 or a portion thereof) and Prosaposin (e.g., PSAP or a portion thereof). The coding sequences of Gcase and Prosaposin are separated by an internal ribosomal entry site (IRES).

FIG. 7 is a schematic depicting one embodiment of an rAAV vector that includes an expression construct encoding a Gcase (e.g., GBA1 or a portion thereof). In this embodiment, the vector comprises a CBA promoter element (CBA), consisting of four parts: the CMV enhancer (CMVe), CBA promoter (CBAp), Exon 1, and intron (int) to constitutively express the codon optimized coding sequence of human GBA1. The 3′ region also contains a WPRE regulatory element followed by a bGH polyA tail. Three transcriptional regulatory activation sites are included at the 5′ end of the promoter region: TATA, RBS, and YY1. The flanking ITRs allow for the correct packaging of the intervening sequences. Two variants of the 5′ ITR sequence (inset box) were evaluated; these have several nucleotide differences within the 20-nucleotide “D” region of wild-type AAV2 ITR. In some embodiments, an rAAV vector contains the “D” domain nucleotide sequence shown on the top line. In some embodiments, an rAAV vector comprises a mutant “D” domain (e.g., an “S” domain, with the nucleotide changes shown on the bottom line).

FIG. 8 is a schematic depicting one embodiment of a plasmid encoding the rAAV vector described in FIG. 7.

FIG. 9A-FIG. 9F show representative data for CBE mouse model validation. Survival (FIG. 9A) was checked 2 times a day and weight (FIG. 9B) was recorded daily and analyzed at P27 (FIG. 9C). All groups started with n=8. Behavior was assessed by latency to fall on rotarod (FIG. 9D) at P24 and by total distance traveled in Open Field (FIG. 9F). Due to early lethality, the number of animals in each group differs: n=8 for PBS and 25 mg/kg CBE, n=4 for the 37.5 mg/kg CBE. The 50 mg/kg CBE treatment group was not assessed in rotarod due to complete lethality by P24. Statistical results are presented for comparisons against the PBS group using ANOVA followed by Tukey's HSD test. Levels of the GCase substrates were analyzed in the cerebral cortex of mice in the PBS and 25 mg/kg CBE treatment groups. Aggregate GluSph and GalSph levels (FIG. 9E) are shown as pmol per mg wet weight of the tissue. Statistical results are presented using student's t-test. Means are presented. Error bars are standard error of the mean (SEM). *P<0.05; **P<0.01; ***P<0.001.

FIG. 10 is a schematic depicting one embodiment of a study design for maximal dose of a rAAV encoding GCase in a CBE mouse model. 4 μL PR001B or dPBS was delivered by ICV injection at P3, and daily 25 mg/kg CBE treatment was initiated at P8. Behavior was assessed in the rotarod assay at P24. Half of the animals were sacrificed at P36, 1 day after their final CBE dose at P35, while the remaining half were sacrificed at P38, 3 days after their final CBE dose at P35. “vg” refers to vector genomes.

FIG. 11A-FIG. 11D show representative data for in-life assessment of maximal PR001B rAAV dose in a CBE mouse model. At P3, mice were treated with either excipient or 8.8e9 vg rAAV via ICV delivery. Daily IP delivery of either PBS or 25 mg/kg CBE was initiated at P8. At the end of the study, half the mice were sacrificed one day after their last CBE dose at P36 (Day 1) while the remaining half went through 3 days of CBE withdrawal before sacrifice at P38 (Day3). All treatment groups (excipient+PBS n=8, rAAV+PBS n=7, excipient+CBE n=8, and rAAV+CBE n=9) were weighed daily (FIG. 11A), and the weight at P33 was analyzed (FIG. 11B). Behavior was assessed by total distance traveled in Open Field at P23 (FIG. 11D) and latency to fall on Rotarod at P24 (FIG. 11C), evaluated for each animal as the median across 3 trials. Due to lethality, n=7 for the excipient+CBE group for the behavioral assays, while n=8 for all other groups. Means across animals are presented. Error bars are SEM. *p<0.05; ***p<0.001, nominal p-values for treatment groups by linear regression in the CBE-treated animals.

FIG. 12A-FIG. 12B show representative data for biochemical assessment of maximal PR001B rAAV dose in a CBE mouse model. The cerebral cortex of all treatment groups was used to measure vector genomes (FIG. 12A) and GCase activity (FIG. 12B). Biodistribution is shown as vector genomes per 1 μg of genomic DNA (gDNA). Dashed line (at 100 vector genomes/μg gDNA) represents the detection threshold for positive vector presence. Enzymatic activity was evaluated by measuring the rate of glucose production by GCase using Amplex Red (Invitrogen; #A22189), then converted to an effective GCase activity level using a recombinant GCase reference standard curve. One unit was defined as the activity of 1 ng/mL of recombinant purified GCase normalized to mg of total protein. n=6-9 per group. Means are presented. Error bars are SEM. *P<0.05; nominal P values for treatment groups in the CBE-treated animals, with collection days and gender corrected for as covariates.

FIG. 12C-FIG. 12D show representative data for glycolipid analysis of maximal PR001B rAAV dose in a CBE mouse model. The cerebral cortex of all treatment groups (PBS+dPBS [left bars in each graph] n=4, CBE+dPBS [center bars in each graph] n=6, and CBE+PR001B [right bars in each graph] n=9) was used to measure GluSph levels (FIG. 12C) and GluCer levels (FIG. 12D) in the groups before (Day 1) or after (Day 3) CBE withdrawal. GluSph and GluCer levels are shown as pmol per nmol of phosphate. Means are presented. Error bars are SEM. *P<0.1; **P<0.01; ***P<0.001, nominal P values for treatment groups in the CBE-treated animals from multiple linear regression, with collection days and gender corrected for as covariates.

FIG. 13 shows representative data for behavioral and biochemical correlations in a CBE mouse model after administration of excipient+PBS, excipient+CBE, and PR001B rAAV+CBE treatment groups. Across treatment groups, performance on Rotarod was negatively correlated with GluCer accumulation (A, p=0.0012 by linear regression), and GluSph accumulation was negatively correlated with increased GCase activity (B, p=0.0086 by linear regression).

FIG. 14 shows representative data for biodistribution of PR001B rAAV in a CBE mouse model. Vector genome presence was quantified by quantitative PCR using a vector reference standard curve; genomic DNA concentration was evaluated by A260 optical density measurement. Dashed line (at 100 vector genomes/μg gDNA) represents the detection threshold for positive vector presence. Means are presented. Error bars are SEM. n=7-9, per group.

FIG. 15A is a schematic depicting one embodiment of a study design for dose-ranging of a rAAV encoding GCase in a CBE mouse model. PR001A was delivered by ICV injection at P3, and daily 25 mg/kg CBE treatment was initiated at P8. Behavior was assessed in the open field and rotarod assays at P21-P22, and by tapered beam at P28. Animals were sacrificed at P38-P40, 1 day after their final CBE dose. The cortices were analyzed for GluSph and GluCer substrate levels and GCase activity. There were 10 mice (5 males, 5 females) in each treatment group.

FIG. 15B-FIG. 15E show representative data for in-life assessment of PR001 rAAV dose-ranging in a CBE mouse model. Mice received excipient or 1 of 3 different doses of PR001A by ICV delivery in 4 μL at P3: low dose (middle bar), medium dose (bar second from right), or high dose (right-most bar). At P8, daily IP treatment of 25 mg/kg CBE was initiated. Mice that received excipient and CBE (bar second from left) or excipient and PBS (left-most bar) served as controls. All treatment groups started with n=10 (5M/5F) per group. All mice were sacrificed 1 day after their final CBE dose (P38-P40). All treatment groups were weighed daily (FIG. 15B), and their weight was analyzed at P37 (FIG. 15C). Motor performance was assessed by latency to fall on rotarod at P24 (FIG. 15D) and latency to traverse the tapered beam at P30 (FIG. 15E). Due to early lethality, the number of mice participating in the behavioral assays was: excipient+PBS (left-most bar) n=10; excipient+CBE (bar second from left) n=9; low dose PR001A+CBE (middle bar) n=6; medium dose PR001A+CBE (bar second from right) n=10; high dose PR001A+CBE (right-most bar) n=7. Means are presented. Error bars are SEM. *P<0.05; **P<0.01 for nominal P values in the CBE-treated groups, with gender corrected for as a covariate.

FIG. 16A shows representative data for biodistribution in a dose-ranging CBE model study of PR001A. Mice received excipient or 1 of 3 different doses of PR001A by ICV delivery at P3: low dose (middle bar), medium dose (bar second from right), or high dose (right-most bar). At P8, daily IP treatment of 25 mg/kg CBE was initiated. Mice that received excipient and CBE (bar second from left) or excipient and PBS (left-most bar) served as controls. All mice were sacrificed at P38-P40, 1 day after their final CBE dose. Presence of vector genomes was assessed in each tissue and all treatment groups, shown as number of vector copies per 1 μg of genomic DNA. Vector genome presence was quantified by qPCR using a vector reference standard curve; N=10, 9, 6, 10, 7 per group, respectively. Dashed lines represent the detection threshold for positive vector presence. Means are presented. Error bars are SEM.

FIG. 16B shows representative data for GCase enzymatic activity in a dose-ranging CBE model study of PR001A. Effective enzymatic GCase activity is shown for each tissue and all treatment groups. Activity is shown as units per mg of total protein with one unit defined as the activity of 1 ng/mL of recombinant purified GCase. Means are presented. Error bars are SEM. Statistical results are presented for comparisons against the excipient+CBE groups (bar second from left). N=10, 9, 6, 10, 7 per group, respectively. * P<0.05; ** P<0.01; *** P<0.001 by ANOVA followed by Tukey's HSD multiple tests correction.

FIG. 16C-FIG. 16D show representative data for glycolipid analysis in a dose-ranging CBE model study of PR001A. GluSph (FIG. 16C) and GluCer (FIG. 16D) levels are shown as pmol per nmol of phosphate. Means are presented. Error bars are SEM. **P<0.01; ***P<0.001 by ANOVA followed by Tukey's HSD multiple tests correction.

FIG. 16E shows representative data for hematoxylin and eosin staining analysis in a dose-ranging CBE model study of PR001A. Brain tissue was processed for staining with hematoxylin and eosin (H&E) and tissue sections were evaluated for pathological changes. The percentage of animals positive for cerebrocortical glial scars, a sign of neuroinflammation, is shown. CBE treatment led to a significant increase in glial scars compared to excipient-treated controls. PR001A significantly reduced CBE-induced glial scarring in a dose-dependent manner. Statistical results are presented for comparisons against the CBE+excipient groups (left bar). N=10, 9, 6, 10, 7 per group, respectively. *: P<0.05; **: P<0.01; ***: P<0.001 for Fischer's exact test.

FIG. 16F shows representative data for cerebrocortical immunohistochemistry analysis in a dose-ranging CBE model study of PR001A. Graph presents the means of immunoreactive area measured within the cerebral cortex (n=5-10 per group). Iba1 (ionizing calcium-binding adaptor molecule 1) immunoreactive area was significantly higher in CBE+excipient-treated animals (bar second from left) than in mice of all other groups investigated. Means are presented and error bars are SEM. Data were analyzed by one-way ANOVA and Sidak's post hoc test for multiple comparisons. **: P<0.01; ***: P<0.001.

FIG. 17 shows representative data for tapered beam analysis in maximal dose GBA1 rAAV in a genetic mouse model. Motor performance of the treatment groups (WT+excipient, n=5), 4L/PS-NA+excipient (n=6), and 4L/PS-NA+rAAV (n=5)) was assayed by Beam Walk 4 weeks post rAAV administration. The total slips and active time are shown as total over 5 trials on different beams. Speed and slips per speed are shown as the average over 5 trials on different beams. Means are presented. Error bars are SEM.

FIG. 18 shows representative data for in vitro expression of rAAV constructs encoding GBA1 in combination with Prosaposin (PSAP), SCARB2, and/or one or more inhibitory nucleic acids. Data indicate transfection of HEK293 cells with each construct resulted in overexpression of the transgenes of interest relative to GFP-transfected cells.

FIG. 19 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIG. 20 shows data for transduction of HEK293 cells using rAAVs having ITRs with wild-type (circles) or alternative (e.g., “outside”; squares) placement of the “D” sequence. The rAAVs having ITRs placed on the “outside” were able to transduce cells as efficiently as rAAVs having wild-type ITRs.

FIG. 21 is a schematic depicting one embodiment of a plasmid comprising an rAAV vector that includes an expression construct encoding Gcase (e.g., GBA1 or a portion thereof).

FIG. 22 is a schematic depicting one embodiment of a plasmid comprising an rAAV vector that includes an expression construct encoding Gcase (e.g., GBA1 or a portion thereof).

FIG. 23 is a schematic depicting one embodiment of a plasmid comprising an rAAV vector that includes an expression construct encoding Gcase (e.g., GBA1 or a portion thereof) and an interfering RNA for α-Syn.

FIG. 24 is a schematic depicting one embodiment of a plasmid comprising an rAAV vector that includes an expression construct encoding Gcase (e.g., GBA1 or a portion thereof) and an interfering RNA for α-Syn.

FIG. 25 is a schematic depicting one embodiment of a plasmid comprising an rAAV vector that includes an expression construct encoding Prosaposin (e.g., PSAP or a portion thereof).

FIG. 26 is a schematic depicting one embodiment of a plasmid comprising an rAAV vector that includes an expression construct encoding Gcase (e.g., GBA1 or a portion thereof).

FIG. 27 is a schematic depicting one embodiment of a plasmid comprising an rAAV vector that includes an expression construct encoding Gcase (e.g., GBA1 or a portion thereof), Prosaposin (e.g., PSAP or a portion thereof), and an interfering RNA for α-Syn.

FIG. 28 shows representative data indicating administration of an rAAV vector encoding Gcase reduces glial scarring in vivo. Tissues were processed for staining with hematoxylin and eosin (H&E) and slides were evaluated for pathological changes. The percentage of animals positive for glial scars, a reflection of reactive astrogliosis, in each group is shown in light shading, while those negative for glial scars are in black. CBE treatment led to a significant increase in glial scars compared to excipient-treated controls. rAAV-GBA1 significantly reduced CBE-induced glial scarring in a dose-dependent manner. Statistical results are presented for comparisons against the Excipient+CBE groups (red). N=10,9,6,10,7 per group, respectively. *: p<0.05; **: p<0.01; ***: p<0.001 for Fischer's exact test.

FIG. 29A-FIG. 29B show representative data for the means of immunofluorescent signal measured within the cortex (n=6-10 per group) of mice administered rAAV-GBA1 or excipient. Quantification of GCase (FIG. 19A) immunolabeling revealed strongest immunofluorescent labeling in high-dose rAAV-GBA1 treated animals, followed by mid- and low-dose rAAV-GBA1 treated animals. Iba1 (FIG. 29B) immunoreactive area was significantly higher in CBE/Excipient treated animals than in mice of all other groups investigated. Data were analyzed with one-way ANOVA and Sidak's post hoc test for multiple comparisons. Bar graphs represent group means+SEM.

FIG. 30 is a bar graph showing representative data for biodistribution of PR001A transgene in study PRV-2018-016 at Day 183. The study is described in Example 12. Transgene levels were analyzed using qPCR methodologies in NHP (non-human primates) 183 days after intra-cisterna magna (ICM) injection of either excipient, low dose of PR001A (6.2×10¹⁰ vg/g brain), or high dose of PR001A (2.3×10¹¹ vg/g brain). Each bar represents the average±SEM of 3 animals per group; values that were below the limit of quantitation were plotted as zero. As the qPCR values for the excipient-treated animals were zero for each region, the excipient bars are not shown on the graph with this scale.

FIG. 31A-FIG. 31B are graphs showing representative data for human GCase expression at day 183 in study PRV-2018-016. The study is described in Example 12. GCase expression levels were determined in NHP (non-human primate) cortex, hippocampus, and midbrain samples that were collected at Day 183 by a Simple Western™ (Jess) analysis. GCase expression levels from NHPs treated with excipient (left in each panel), low dose of PR001A (6.2×10¹⁰ vg/g brain weight; center in each panel) or high dose of PR001A (2.3×10¹¹ vg/g brain weight; right in each panel) are shown. FIG. 31A presents the data from individual cortex, hippocampus, and midbrain regions. FIG. 31B shows the percent change from the excipient-treated group (left), low dose (center), and high dose (right) groups. The data for this plot was mean-normalized within tissue and combined for cortex, hippocampus, and midbrain. Each bar represents percent of the excipient group median for each dose of the mean normalized data. To calculate significance, a one-way ANOVA was performed to consider significance for a combined treatment group that included both the low- and high-dose treated animals to the excipient group. P value=0.014 (*<0.05).

FIG. 32 is a series of plots showing representative data for biodistribution of PR001A transgene quantified by qPCR in Study PRV-2019-005. The study is described in Example 12. Transgene levels were analyzed using qPCR methodologies in NHPs (non-human primates) 30 and 90 days after intra-cisterna magna (ICM) injection of either excipient or PR001A (7.0×10¹¹ vg/g brain). Each plot represents each individual animal (n=3/group) with the average±SEM.

FIG. 33 is a line graph showing representative data for GCase activity after in vitro transduction of HEK293T cells with PR001A. HEK293T cells transduced with PR001A at different multiplicity of infection (MOI) were assayed for GCase activity. Activity was measured by hydrolysis of the pro-fluorescent substrate resorufin P-D-glucopyranoside. Fluorescence of the cleaved substrate was determined using a plate reader at an excitation of 573 nm and an emission of 610 nm. Values are means±SEM, n=2; a unit is equivalent to the activity of 1 ng/mL of recombinant purified GCase.

FIG. 34A-FIG. 34B are bar graphs showing representative data for GCase activity (FIG. 34A) and α-Synuclein levels (FIG. 34B) after in vitro transduction of HeLa cells with PR001A. HeLa cells treated with excipient (left bar) or transduced with 2×10⁵ vg/cell PR001A (center bar) or 2×10⁶ vg/cell PR001A (right bar) were collected 72 hours post treatment and analyzed for GCase activity levels (FIG. 34A) by a fluorometric enzyme assay or α-Synuclein levels (FIG. 34B) by ELISA. Effective enzymatic GCase activity is shown as units per mg of total protein with one unit defined as the activity of 1 ng/mL of recombinant purified GCase. α-Synuclein concentration is presented as ng/mL per mg of total protein. Studies were performed in biological triplicate. Means are presented. Error bars are SEM. One-way ANOVA was used followed by Dunnett's multiple comparisons test.

FIG. 35A-FIG. 35B are bar graphs showing representative data for GCase activity (FIG. 35A) and α-Synuclein levels (FIG. 35B) after in vitro transduction of mouse hippocampal neurons with PR001A. Primary cultures of mouse hippocampal neurons were treated with excipient (left bar) or transduced with 1.3×10⁵ vg/cell PR001A (center bar) or 1.3×10⁶ vg/cell PR001A (right bar) on Day in vitro (DIV) 2. On DIV9, cells were collected and analyzed for GCase activity levels (FIG. 35A) by a fluorometric enzyme assay or α-Synuclein levels (FIG. 35B) by ELISA. GCase activity is shown as relative fluorescent units (RFU) per hour per mg of total protein. α-Synuclein concentration is presented as ng/mL per mg of total protein. Studies were performed in biological duplicate. Means are presented. Error bars are SEM. One-way ANOVA was used followed by Dunnett's multiple comparisons test.

FIG. 36 is a schematic depicting one embodiment of a study design for long-term treatment with a rAAV encoding GCase in a CBE mouse model. PR001A was delivered by ICV injection at P3, and daily CBE treatment was initiated at P8. Behavior was assessed in the rotarod assay at Weeks 3, 6, and 15 and the tapered beam assay at Weeks 4, 7, 13. The animals were sacrificed around Week 26, 1 day after their final CBE dose. The cerebral cortices were analyzed for GluSph and GluCer substrate levels and GCase activity. There were 10-11 animals per treatment group, each including male and female mice.

FIG. 37A-FIG. 37D show representative data for assessment of long-term PR001A treatment in a CBE model. The cortex of all treatment groups (PBS+excipient: left bar, CBE+excipient: center bar, CBE+2.0×10¹⁰ vg PR001A: right bar) was used to measure vector genomes (FIG. 37A), GCase activity (FIG. 37B), GluSph levels (FIG. 37C), and GluCer levels (FIG. 37D). Presence of vector genomes was assessed in each tissue and all treatment groups, shown as number of vector copies per 1 μg of genomic DNA. Vector genome presence was quantified by qPCR using a vector reference standard curve. Effective enzymatic GCase activity is shown as units per mg of total protein with one unit defined as the activity of 1 ng/mL of recombinant purified GCase. GluSph, and GluCer levels are shown as pmol per nmol of phosphate. Means are presented. Error bars are SEM. N=10, 11, 10 per group, respectively. (*) P<0.1, *P<0.05; **P<0.01; ***P<0.001 by ANOVA followed by Tukey's HSD multiple tests correction.

FIG. 38A-FIG. 38E show representative data for in-life assessment of additional dose-ranging PR001A in a CBE model. All treatment groups were weighed daily (FIG. 38A), and their weight was analyzed at P45 (FIG. 38B). Motor performance was assessed by latency to fall on rotarod at Week 3 (FIG. 38C) and Week 5 (FIG. 38D), and latency to traverse the tapered beam (FIG. 38E) at Week 4. n=8-11 per group. Means are presented. Error bars are SEM. Statistical results are presented for comparisons against the CBE+excipient group (second bar from left). ***P<0.001 by ANOVA followed by Tukey's HSD test.

FIG. 39 shows representative data for biodistribution in an additional dose-ranging CBE model study. Presence of vector genomes was assessed in each tissue and all treatment groups, shown as number of vector copies per 1 μg of gDNA. Vector genome presence was quantified by qPCR using a vector reference standard curve; n=9-11 per group. Black dashed line (at 100 vector genomes/μg gDNA) represents the detection threshold for positive vector presence. Means are presented. Error bars are SEM.

FIG. 40 shows representative data for GCase enzymatic activity in an additional dose-ranging CBE model study. Effective enzymatic GCase activity was measured and is shown for the cerebral cortex of all treatment groups. Activity is shown as units per mg of total protein with one unit defined as the activity of 1 ng/mL of recombinant purified GCase. Means are presented. Error bars are SEM. Statistical results are presented for comparisons against the CBE+excipient group (second bar from left). n=9-11 per group. (*)P<0.1 by ANOVA followed by Tukey's HSD test.

FIG. 41A-FIG. 41B show representative data for glycolipid analysis in an additional dose-ranging CBE model study. GluSph (FIG. 41A) and GluCer (FIG. 41B) levels are shown as pmol per nmol of phosphate. Means are presented. Error bars are SEM. Statistical results are presented for comparisons against the CBE+excipient group (second bar from left). n=9-11 per group. **P<0.01; ***P<0.001, by ANOVA followed by Tukey's HSD test.

FIG. 42A-FIG. 42D show representative data for in-life assessment of further dose-ranging PR001A in a CBE model study. All treatment groups were weighed daily (FIG. 42A), and their weight was analyzed at P37 (FIG. 42B). Motor performance was assessed by latency to fall on rotarod at Week 3 (FIG. 42C) and latency to traverse the tapered beam (FIG. 42D) at Week 4. n=9-10 per group. Means are presented. Error bars are SEM. Statistical results are presented for comparisons against the CBE+excipient group (second bar from left). ***P<0.001 by ANOVA followed by Tukey's HSD test.

FIG. 43A-FIG. 43B show representative data for biodistribution and GCase enzymatic activity in further dose-ranging PR001A in a CBE model study. Vector genomes were measured in the cerebral cortex (FIG. 43A) of all treatment groups and are shown as number of vector copies per 1 μg of genomic DNA (gDNA). Vector genome presence was quantified by qPCR using a vector reference standard curve. Black dashed line represents the detection threshold for positive vector presence (at 100 vector genomes/μg gDNA). Effective enzymatic GCase activity was measured in the cerebral cortex (FIG. 43B) and is shown as units per mg of total protein with one unit defined as the activity of 1 ng/mL of recombinant purified GCase. Means are presented. Error bars are SEM. N=9-10 per group. ***P<0.001; by ANOVA followed by Tukey's HSD test.

FIG. 44A-FIG. 44B show representative data for glycolipid analysis in further dose-ranging PR001A in a CBE model study. GluSph (FIG. 44A) and GluCer (FIG. 44B) levels are shown as pmol per nmol of phosphate. Means are presented. Error bars are SEM. Statistical results are presented for comparisons against the CBE+excipient group (second bar from left). n=9-10 per group. ***P<0.001 by ANOVA followed by Tukey's HSD test.

FIG. 45 is a schematic depicting one embodiment of a study design for treatment with a rAAV encoding GCase in a 4L/PS-NA genetic mouse model. PR001A was delivered by ICV injection to 4L/PS-NA mice at 3-4 weeks of age. Beam walk was tested at Weeks 8, 12, and 18 of life (5, 9, and 15 weeks post-ICV treatment) and rotarod was tested at Weeks 12 and 18 of life (9 and 15 weeks post-ICV treatment). Mice were sacrificed at Week 18. The cerebral cortices were analyzed for GCase enzymatic activity and the cerebella were analyzed for GluSph and GluCer substrate levels. There were 3 male and 3 female mice in each treatment group.

FIG. 46 shows representative data for biodistribution in maximal dose PR001A in a 4L/PS-NA genetic mouse model. Presence of vector genomes was assessed in each tissue and all treatment groups, shown as number of vector copies per 1 μg of genomic DNA (gDNA). Vector genome presence was quantified by qPCR using a vector reference standard curve. Means are presented. Error bars are SEM. n=4-5 per group. Dashed lines represent the detection threshold for positive vector presence (at 100 vector genomes/μg gDNA).

FIG. 47 shows representative data for GCase enzymatic activity in maximal dose PR001A in a 4L/PS-NA genetic mouse model. Effective enzymatic GCase activity was measured and is shown for each tissue and all treatment groups. Activity is shown as units per mg of total protein with one unit defined as the activity of 1 ng/mL of recombinant purified GCase. Means are presented. Error bars are SEM. N=4-5 per group. *: P<0.05; **: P<0.01; ***: P<0.001 by ANOVA followed by Tukey's HSD multiple tests correction.

FIG. 48A-FIG. 48B show representative data for glycolipid analysis of PR001A in a 4L/PS-NA genetic mouse model. 4L/PS-NA mice received excipient (center bar) or 1.5×10¹⁰ vg PR001A (right bar), and control mice received excipient (left bar) by ICV delivery at postnatal Day P23. The cerebellum was used to measure GluSph (FIG. 48A) and GluCer (FIG. 48B) levels. Levels are shown as pmol per nmol of phosphate. Means are presented. Error bars are SEM. n=4,5,5 per group, respectively. *: P<0.05; **: P<0.01; ***: P<0.001 by ANOVA followed by Tukey's HSD multiple tests correction.

FIG. 49A-FIG. 49B show representative data for biochemical assessment of cerebral cortex α-Synuclein accumulation in a 4L/PS-NA genetic mouse model. 4L/PS-NA mice received ICV excipient (center bar) or 1.5×10¹⁰ vg PR001A (right bar), and control mice received ICV excipient (left bar) at postnatal Day 23. The Triton X-soluble and Triton X-insoluble fractions of brain lysates from the cerebral cortex were analyzed for α-Synuclein protein levels using a customized immunosorbent assay. Insoluble α-Synuclein (FIG. 49A) and the ratio of insoluble to soluble α-Synuclein (FIG. 49B) are shown. Means are presented. Error bars are SEM. N=3-5 per group. (*): P<0.20, ANOVA followed by Tukey's HSD multiple tests correction.

FIG. 50 is a schematic depicting one embodiment of a study design for dose-ranging PR001A rAAV in a 4L/PS-NA genetic mouse model. PR001A was delivered by ICV injection to 4L/PS-NA mice at 3-4 weeks. Beam walk was assessed at Weeks 8, 12, and 18 of life (5, 9, and 15 weeks post-ICV treatment) and rotarod was assessed at Weeks 12 and 18 of life (9 and 15 weeks post-ICV treatment). Mice were sacrificed at Week 18. The cerebral cortices were analyzed for GCase enzymatic activity, and the cerebella were analyzed for GluSph and GluCer substrate levels. There were 10-11 mice per treatment group.

FIG. 51 shows representative data for week 18 behavioral analyses in dose-ranging PR001A in a 4L/PS-NA genetic mouse model. Motor performance in beam walk was evaluated, and the average total slips per speed are shown over 2 trials on different beams. N=10, 6, 5, 7, 4, 8 for group, respectively. Means are presented. Error bars are SEM; *: P<0.05; ***: P<0.001 by ANOVA followed by Tukey's HSD multiple tests correction.

FIG. 52A-FIG. 52B show representative data for biodistribution and GCase enzymatic activity in dose-ranging PR001A in a 4L/PS-NA genetic mouse model. Vector genomes were measured in the cerebral cortex (FIG. 52A) of all treatment groups and are shown as number of vector copies per 1 μg of genomic DNA (gDNA). Vector genome presence was quantified by qPCR using a vector reference standard curve. Dashed line represents the detection threshold for positive vector genome presence (at 100 vector genomes/μg gDNA). Effective enzymatic GCase activity was measured in the cerebral cortex (FIG. 52B) and is shown as units per mg of total protein with one unit defined as the activity of 1 ng/mL of recombinant purified GCase. Means are presented. Error bars are SEM. n=10, 10, 10, 10, 7, 8 per group, respectively. ***P<0.001 by ANOVA followed by Tukey's HSD multiple tests correction.

FIG. 53A-FIG. 53B show representative data for glycolipid analysis in dose-ranging PR001A in a 4L/PS-NA genetic mouse model. The cerebellum was used to measure GluSph (FIG. 53A) and GluCer (FIG. 53B) levels. Levels are shown as pmol per nmol of phosphate. Means are presented. Error bars are SEM. n=10, 10, 10, 10, 7, 8 per group, respectively. ***: P<0.001 by ANOVA followed by Tukey's HSD multiple tests correction. (#): P<0.1; #: P<0.05 by multiple linear regression for genotype and dose across all animals.

FIG. 54A-FIG. 54B show representative data for biochemical assessment of α-Synuclein protein levels in CBE-treated α-Synuclein transgenic mice. Hippocampal brain lysates were analyzed for α-Synuclein concentration using the Simple Western™ (Jess) automated capillary western blot system and the MJFR-14-6-4-2 α-Synuclein antibody. Multiple bands were observed between 48 kDa and 230 kDa and were grouped as “high molecular weight” (UMW). A single band was present at 18 kDa, consistent with the predicted molecular weight of α-Synuclein monomer. Mean fold change over the normalized mean of the A53T+excipient group is presented. Error bars are SEM. N=3-5 per group. *: P<0.05 by ANOVA followed by Tukey's HSD multiple test correction.

FIG. 55 is a schematic depicting one embodiment of a plasmid encoding a recombinant adeno-associated virus vector (PR001A) comprising an expression construct encoding human Gcase. “bp” refers to “base pairs”. “kan” refers to a gene that confers resistance to kanamycin. “ORF1” refers to an open reading frame for Gcase. “ITR” refers to an adeno-associated virus inverted terminal repeat sequence. “TRY” refers to a sequence comprising three transcriptional regulatory activation sites: TATA, RBS, and YY1. “CBAp” refers to a chicken β-actin promoter. “CMVe” refers to a cytomegalovirus enhancer. “WPRE” refers to a woodchuck hepatitis virus post-transcriptional regulatory element. “bGH” refers to a bovine Growth Hormone polyA signal tail. “int” refers to an intron. The nucleotide sequences of the two strands of PR001A are provided in SEQ ID NOs: 39 and 40.

FIG. 56 is a schematic depicting one embodiment of a plasmid encoding a recombinant adeno-associated virus vector (PR004X) comprising an expression construct encoding human Gcase and a shRNA targeting α-Synuclein. “bp” refers to “base pairs”. “kan” refers to a gene that confers resistance to kanamycin. “aSyn MshRNA” refers to a region encoding a shRNA inhibiting α-Synuclein. “GBA_CDSopt” refers to an open reading frame for Gcase. “ITR” refers to an adeno-associated virus inverted terminal repeat sequence. “TRY” refers to a sequence comprising three transcriptional regulatory activation sites: TATA, RBS, and YY1. “CBAp” refers to a chicken β-actin promoter. “CMVe” refers to a cytomegalovirus enhancer. “WPRE” refers to a woodchuck hepatitis virus post-transcriptional regulatory element. “bGH” refers to a bovine Growth Hormone polyA signal tail. “int” refers to an intron. The nucleotide sequences (sequence verified) of the two strands of PR004X are provided in SEQ ID NOs: 41 and 42.

FIG. 57 is a schematic depicting one embodiment of a plasmid encoding a recombinant adeno-associated virus vector (PR004Y) comprising an expression construct encoding human Gcase and a shRNA targeting α-Synuclein. “bp” refers to “base pairs”. “kan” refers to a gene that confers resistance to kanamycin. “shSNCA” refers to a region encoding a shRNA inhibiting α-Synuclein. “GBA_CDSopt” refers to an open reading frame for Gcase. “ITR” refers to an adeno-associated virus inverted terminal repeat sequence. “TRY” refers to a sequence comprising three transcriptional regulatory activation sites: TATA, RBS, and YY1. “CBAp” refers to a chicken β-actin promoter. “CMVe” refers to a cytomegalovirus enhancer. “WPRE” refers to a woodchuck hepatitis virus post-transcriptional regulatory element. “bGH” refers to a bovine Growth Hormone polyA signal tail. “int” refers to an intron. The nucleotide sequences (theoretical) of the two strands of PR004Y are provided in SEQ ID NOs: 43 and 44.

FIG. 58 is a schematic depicting one embodiment of a plasmid encoding a recombinant adeno-associated virus vector (PR014X) comprising an expression construct encoding a shRNA targeting α-Synuclein. “bp” refers to “base pairs”. “kan” refers to a gene that confers resistance to kanamycin. “aSyn_MshRNA” refers to a region encoding a shRNA inhibiting α-Synuclein. “ITR” refers to an adeno-associated virus inverted terminal repeat sequence. “TRY” refers to a sequence comprising three transcriptional regulatory activation sites: TATA, RBS, and YY1. “CBAp” refers to a chicken β-actin promoter. “CMVe” refers to a cytomegalovirus enhancer. “WPRE” refers to a woodchuck hepatitis virus post-transcriptional regulatory element. “bGH” refers to a bovine Growth Hormone polyA signal tail. “int” refers to an intron. The nucleotide sequences (theoretical) of the two strands of PR014X are provided in SEQ ID NOs: 45 and 46. The nucleotide sequences (theoretical) of the two strands of the region encoding the shRNA are provided in SEQ ID NOs: 47 and 48.

FIG. 59 is a schematic depicting one embodiment of a study design for dose-ranging PR001 rAAV in a D409V Hom genetic mouse model. PR001 was delivered by intravenous (IV) injection to D409V Hom mice. The parameters listed in the figure were assessed 5 weeks later.

FIG. 60A-FIG. 60C show representative data for liver biochemistry of PR001 intravenous (IV) administration in a D409V Hom genetic mouse model. Mice were sacrificed 5-weeks post-IV injection. Cytokine levels (FIG. 60A) and glycolipid levels (FIG. 60B; FIG. 60C) were quantified. Statistics were determined using ANOVA followed by Dunnett's test comparing to the D409V Hom+Excipient group. Means are presented +/−SEM (n=8-10/group). ****: p<0.0001; ***: p<0.001; **: p<0.01; *: p<0.05; (*): p=0.10. GluCer =glucosylceramide. GluSph=glucosylsphingosine. WT=wild type.

FIG. 61A-FIG. 61B show representative data for brain biochemistry of PR001 intravenous (IV) administration in a D409V Hom genetic mouse model. Mice were sacrificed 5-weeks post-IV injection. Glycolipid levels were quantified. Statistics were determined using ANOVA followed by Dunnett's test comparing to the D409V Hom+Excipient group. Means are presented +/−SEM (n=8-10/group). ****: p<0.0001; *: p<0.05. GluCer=glucosylceramide. GluSph=glucosylsphingosine. WT=wild type.

FIG. 62 shows representative data for lung biochemistry of PR001 intravenous (IV) administration in a D409V Hom genetic mouse model. Mice were sacrificed 5-weeks post-IV injection. Cytokine levels were quantified. Statistics were determined using ANOVA followed by Dunnett's test comparing to the D409V Hom+Excipient group. Means are presented +/−SEM (n=8-10/group). *: p<0.05. WT=wild type.

FIG. 63 is a schematic depicting one embodiment of a study design for dose-ranging PR001 rAAV in a 4L/PS-NA genetic mouse model. PR001 was delivered by intravenous (IV) injection to 4L/PS-NA mice. The parameters listed in the figure were assessed at the time points shown.

FIG. 64A-FIG. 64B show representative data for liver biochemistry of PR001 intravenous (IV) administration in a 4L/PS-NA genetic mouse model. Mice were sacrificed 15-weeks post-IV injection. Glycolipid levels were quantified. Statistics were determined using ANOVA followed by Dunnett's test comparing to the 4L/PS-NA+Excipient group. Means are presented +/−SEM (n=10/group). ****: p<0.0001; ***: p<0.001; *: p<0.05. GluCer=glucosylceramide. GluSph=glucosylsphingosine.

FIG. 65A-FIG. 65B show representative data for brain biochemistry of PR001 intravenous (IV) administration in a 4L/PS-NA genetic mouse model. Mice were sacrificed 15-weeks post-IV injection. Glycolipid levels were quantified. Statistics were determined using ANOVA followed by Dunnett's test comparing to the 4L/PS-NA+Excipient group. Means are presented +/−SEM (n=10/group). ****: p<0.0001; **: p<0.01; *: p<0.05. GluCer=glucosylceramide. GluSph=glucosylsphingosine.

FIG. 66A-FIG. 66B show representative data for α-Synuclein protein levels and Gcase activity in HeLa cells after transduction with PR004 or PR014. HeLa cells were treated with PR004, PR014 or excipient, and α-Synuclein levels (FIG. 66A) and GCase activity (FIG. 66B) in cell lysates were measured after 72 hours. Data is presented as mean±SEM (n=3/condition).

FIG. 67A-FIG. 67C show representative data for PR004 efficacy in neuronal cultures from Parkinson's disease patient-derived induced pluripotent stem cells (iPSC). Induced pluripotent stem cells derived from a Parkinson's disease patient with a SNCA triplication were differentiated into neurons (FIG. 67A). iPSC-derived neurons were treated with PR004 or excipient, and GCase activity (FIG. 67B) and α-Synuclein levels (FIG. 67C) were measured in cell lysates after two weeks. Statistics determined by unpaired t-test; *=p<0.05, **=p<0.01. Data is presented as mean±SEM (n=2-3/group).

FIG. 68A-FIG. 68B show representative data for studies assessing shRNA targeting SNCA from the PR004 vector in HEK293 cells by qRT-PCR. HEK293 cells were transfected with PR004 or control, and RNA was extracted after 72 hours. qRT-PCR for various genes was performed and normalized to GAPDH expression. Data is normalized to the control condition and presented as mean±SEM (n=3/group).

FIG. 69 is a schematic depicting one embodiment of a study design examining gastrointestinal, motor behavior, and biochemical endpoints in the SNCA-A53T PAC mouse model after administration of PR004. ICV=intracerebroventricular.

FIG. 70 is a schematic depicting one embodiment of a study design examining motor behavior and biochemical analysis in the AAV2-SNCA-A53T IPa injection mouse model after administration of PR004. IPa=intraparenchymal. SN=substantia nigra. ICV=intracerebroventricular.

FIG. 71A-FIG. 71B show representative data for studies assessing motor phenotypes after PR004 administration in the AAV2-SNCA-A53T mouse model. Ten-week old mice were dosed with (1) AAV-Null or AAV-SNCA-A53T via IPa injection to the SN, and (2) excipient or PR004 via ICV injection. Fine motor kinematic gait analysis (MotoRater) was performed at 4 weeks (FIG. 71A) and 9 weeks (FIG. 71B) after treatment. Statistics determined by ANOVA followed by Dunnett's multiple tests correction; *=p<0.05, **=p<0.01. Data is presented as mean±SEM (n=10/group). IPa=intraparenchymal. SN=substantia nigra. ICV=intracerebroventricular.

### DETAILED DESCRIPTION

The disclosure is based, in part, on compositions and methods for expression of combinations of certain gene products (e.g., gene products associated with CNS disease) in a subject. A gene product can be a protein, a fragment (e.g., portion) of a protein, an interfering nucleic acid that inhibits a CNS disease-associated gene, etc. In some embodiments, a gene product is a protein or a protein fragment encoded by a CNS disease-associated gene. In some embodiments, a gene product is an interfering nucleic acid (e.g., shRNA, siRNA, miRNA, amiRNA, etc.) that inhibits a CNS disease-associated gene.

A CNS disease-associated gene refers to a gene encoding a gene product that is genetically, biochemically or functionally associated with a central nervous system (CNS) disease, such as Parkinson's disease (PD), Gaucher disease (GD) or a synucleinopathy. For example, individuals having mutations in the GBA1 gene (which encodes the protein Gcase), have been observed to be have an increased risk of developing PD compared to individuals that do not have a mutation in GBA1. In another example, PD is associated with accumulation of protein aggregates comprising α-Synuclein (α-Syn) protein; accordingly, SNCA (which encodes α-Syn) is a CNS disease-associated gene. In some embodiments, an expression cassette described herein encodes a wild-type or non-mutant form of a CNS disease-associated gene (or coding sequence thereof). Examples of CNS disease-associated genes are listed in Table 1.

TABLE 1
Examples of CNS disease-associated genes

| | | | NCBI Accession |
|---|---|---|---|
| Name | Gene | Function | No. |
| Lysosome membrane | SCARB2/LIMP2 | lysosomal receptor for | NP_005497.1 |
| protein 2 | | glucosylceramidase | (Isoform 1), |
| | | (GBA targeting) | NP_001191184.1 |
| | | | (Isoform 2) |
| Prosaposin | PSAP | precursor for saposins | AAH01503.1, |
| | | A, B, C, and D, which | AAH07612.1, |
| | | localize to the | AAH04275.1, |
| | | lysosomal compartment | AAA60303.1 |
| | | and facilitate the | |
| | | catabolism of | |
| | | glycosphingolipids with | |
| | | short oligosaccharide | |
| | | groups | |
| beta-Glucocerebrosidase | GBA1 | cleaves the beta- | NP_001005742.1 |
| | | glucosidic linkage of | (Isoform 1), |
| | | glucocerebroside | NP_001165282.1 |
| | | | (Isoform 2), |
| | | | NP_001165283.1 |
| | | | (Isoform 3) |
| alpha-Synuclein | SNCA | plays a role in | NP_001139527.1 |
| | | maintaining a supply | |
| | | of synaptic vesicles in | |
| | | presynaptic terminals | |
| | | by clustering synaptic | |
| | | vesicles, and may help | |
| | | regulate the release of | |
| | | dopamine | |

Deficits in enzymes such as lysosomal acid β-glucocerebrosidase (e.g., the gene product of GBA1 gene; also referred to as GCase), as well as common variants in many genes implicated in lysosome function or trafficking of macromolecules to the lysosome (e.g., Lysosomal Membrane Protein 1 (LIMP), also referred to as SCARB2), have been associated with increased PD risk and/or increased risk of Gaucher disease (e.g., neuronopathic Gaucher disease, such as Type 2 Gaucher disease or Type 3 Gaucher disease). The disclosure is based, in part, on expression constructs (e.g., vectors) encoding Gcase (or a portion thereof), prosaposin (or a portion thereof), LIMP2 (or a portion thereof), or a combination of Gcase (or a portion thereof) and one or more additional gene products from genes (e.g., LIMP2, Prosaposin, and/or α-Synuclein (α-Syn)) associated with central nervous system (CNS) diseases, for example PD, Gaucher disease, etc. In some embodiments, combinations of gene products described herein act together (e.g., synergistically) to reduce one or more signs and symptoms of a CNS disease when expressed in a subject.

Accordingly, in some aspects, the disclosure provides an isolated nucleic acid comprising an expression construct encoding a Gcase (e.g., the gene product of GBA1 gene). In some embodiments, the isolated nucleic acid comprises a Gcase-encoding sequence that has been codon optimized (e.g., codon optimized for expression in mammalian cells, for example human cells). In some embodiments, the nucleic acid sequence encoding the Gcase encodes a protein comprising an amino acid sequence as set forth in SEQ ID NO: 14 (e.g., as set forth in NCBI Reference Sequence NP_000148.2). In some embodiments, the isolated nucleic acid comprises the sequence set forth in SEQ ID NO: 15. In some embodiments the expression construct comprises adeno-associated virus (AAV) inverted terminal repeats (ITRs), for example AAV ITRs flanking the nucleic acid sequence encoding the Gcase.

In some aspects, the disclosure provides an isolated nucleic acid comprising an expression construct encoding Prosaposin (e.g., the gene product of PSAP gene). In some embodiments, the isolated nucleic acid comprises a prosaposin-encoding sequence that has been codon optimized (e.g., codon optimized for expression in mammalian cells, for example human cells). In some embodiments, the nucleic acid sequence encoding the prosaposin encodes a protein comprising an amino acid sequence as set forth in SEQ ID NO: 16 (e.g., as set forth in NCBI Reference Sequence NP_002769.1). In some embodiments, the isolated nucleic acid comprises the sequence set forth in SEQ ID NO: 17. In some embodiments the expression construct comprises adeno-associated virus (AAV) inverted terminal repeats (ITRs), for example AAV ITRs flanking the nucleic acid sequence encoding the prosaposin.

In some aspects, the disclosure provides an isolated nucleic acid comprising an expression construct encoding LIMP2/SCARB2 (e.g., the gene product of SCARB2 gene). In some embodiments, the isolated nucleic acid comprises a SCARB2-encoding sequence that has been codon optimized (e.g., codon optimized for expression in mammalian cells, for example human cells). In some embodiments, the nucleic acid sequence encoding the LIMP2/SCARB2 encodes a protein comprising an amino acid sequence as set forth in SEQ ID NO: 18 (e.g., as set forth in NCBI Reference Sequence NP_005497.1). In some embodiments, the isolated nucleic acid comprises the sequence set forth in SEQ ID NO: 29. In some embodiments the expression construct comprises adeno-associated virus (AAV) inverted terminal repeats (ITRs), for example AAV ITRs flanking the nucleic acid sequence encoding the SCARB2.

In some aspects, the disclosure provides an isolated nucleic acid comprising an expression construct encoding a first gene product and a second gene product, wherein each gene product independently is selected from the gene products, or portions thereof, set forth in Table 1.

In some embodiments, a first gene product or a second gene product is a Gcase protein, or a portion thereof. In some embodiments, a first gene product or a second gene product is LIMP2 or a portion thereof, or Prosaposin or a portion thereof. In some embodiments, the first gene product is a Gcase protein, and the second gene product is LIMP2 or a portion thereof, or Prosaposin or a portion thereof.

In some embodiments, an expression construct encodes (e.g., alone or in addition to another gene product) an interfering nucleic acid (e.g., shRNA, miRNA, dsRNA, etc.). In some embodiments, an interfering nucleic acid inhibits expression of α-Synuclein (α-Synuclein). In some embodiments, an interfering nucleic acid that targets α-Synuclein comprises a sequence set forth in any one of SEQ ID NOs: 20-25. In some embodiments, an interfering nucleic acid that targets α-Synuclein comprises a sequence set forth in SEQ ID NO: 20. In some embodiments, an interfering nucleic acid that targets α-Synuclein binds to (e.g., hybridizes with) a sequence set forth in any one of SEQ ID NO: 20-25. In some embodiments, an interfering nucleic acid that targets α-Synuclein binds to (e.g., hybridizes with) a sequence set forth in SEQ ID NO: 20.

In some embodiments, an expression construct further comprises one or more promoters. In some embodiments, a promoter is a chicken-beta actin (CBA) promoter, a CAG promoter, a CD68 promoter, or a JeT promoter. In some embodiments, a promoter is a RNA pol II promoter or an RNA pol III promoter (e.g., U6, etc.).

In some embodiments, an expression construct further comprises an internal ribosomal entry site (IRES). In some embodiments, an IRES is located between a first gene product and a second gene product.

In some embodiments, an expression construct further comprises a self-cleaving peptide coding sequence. In some embodiments, a self-cleaving peptide is a T2A peptide.

In some embodiments, an expression construct comprises two adeno-associated virus (AAV) inverted terminal repeat (ITR) sequences. In some embodiments, ITR sequences flank a first gene product and a second gene product (e.g., are arranged as follows from 5′-end to 3′-end: ITR-first gene product-second gene product-ITR). In some embodiments, one of the ITR sequences of an isolated nucleic acid lacks a functional terminal resolution site (trs). For example, in some embodiments, one of the ITRs is a ΔITR.

The disclosure relates, in some aspects, to rAAV vectors comprising an ITR having a modified “D” region (e.g., a D sequence that is modified relative to wild-type AAV2 ITR, SEQ ID NO: 29). In some embodiments, the ITR having the modified D region is the 5′ ITR of the rAAV vector. In some embodiments, a modified “D” region comprises an “S” sequence, for example as set forth in SEQ ID NO: 26. In some embodiments, the ITR having the modified “D” region is the 3′ ITR of the rAAV vector. In some embodiments, a modified “D” region comprises a 3′ITR in which the “D” region is positioned at the 3′ end of the ITR (e.g., on the outside or terminal end of the ITR relative to the transgene insert of the vector). In some embodiments, a modified “D” region comprises a sequence as set forth in SEQ ID NO: 26 or 27.

In some embodiments, an isolated nucleic acid (e.g., an rAAV vector) comprises a TRY region. In some embodiments, a TRY region comprises the sequence set forth in SEQ ID NO: 28.

In some embodiments, an isolated nucleic acid described by the disclosure comprises or consists of the sequence set forth in any one of SEQ ID NOs: 1 to 13, 15, 17, 19, and 32-48. In some embodiments, an isolated nucleic acid described by the disclosure encodes a peptide comprising or consisting of the sequence set forth in any one of SEQ ID NOs: 14, 16, and 18.

In some aspects, the disclosure provides a vector comprising an isolated nucleic acid as described by the disclosure. In some embodiments, a vector is a plasmid, or a viral vector. In some embodiments, a viral vector is a recombinant AAV (rAAV) vector. In some embodiments, an rAAV vector is single-stranded (e.g., single-stranded DNA).

In some aspects, the disclosure provides a host cell comprising an isolated nucleic acid as described by the disclosure or a vector as described by the disclosure.

In some aspects, the disclosure provides a recombinant adeno-associated virus (rAAV) comprising a capsid protein and an isolated nucleic acid or a vector as described by the disclosure.

In some embodiments, a capsid protein is capable of crossing the blood-brain barrier, for example an AAV9 capsid protein or an AAVrh.10 capsid protein. In some embodiments, an rAAV transduces neuronal cells and non-neuronal cells of the central nervous system (CNS).

In some aspects, the disclosure provides a method for treating a subject having or suspected of having or suspected of having a central nervous system (CNS) disease, the method comprising administering to the subject a composition (e.g., a composition comprising an isolated nucleic acid or a vector or a rAAV) as described by the disclosure. In some embodiments, the CNS disease is a neurodegenerative disease, such as a neurodegenerative disease listed in Table 4. In some embodiments, the CNS disease is a synucleinopathy, such as a synucleinopathy listed in Table 5. In some embodiments, the CNS disease is a tauopathy, such as a tauopathy listed in Table 6. In some embodiments, the CNS disease is a lysosomal storage disease, such as a lysosomal storage disease listed in Table 7. In some embodiments, the lysosomal storage disease is neuronopathic Gaucher disease, such as Type 1 Gaucher disease, Type 2 Gaucher disease or Type 3 Gaucher disease.

In some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

In some embodiments, the disclosure provides a method for treating a subject having Type 2 Gaucher disease or Type 3 Gaucher disease, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the rAAV is administered to a subject having Type 2 Gaucher disease or Type 3 Gaucher disease at a dose of about 1.3×10¹¹ vector genomes (vg)/g brain.

In some embodiments, the disclosure provides a method for treating a subject having Parkinson's disease with a glucocerebrosidase-1 (GBA1) mutation, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the rAAV is administered to a subject having Parkinson's disease at a dose of about 1×10¹⁴ vector genomes (vg) or about 2×10¹⁴ vg.

In some embodiments, the rAAV is administered via a suboccipital injection into the cisterna magna.

In some embodiments, a composition comprises a nucleic acid (e.g., an rAAV genome, for example encapsidated by AAV capsid proteins) that encodes two or more gene products (e.g., CNS disease-associated gene products), for example 2, 3, 4, 5, or more gene products described in this application. In some embodiments, a composition comprises two or more (e.g., 2, 3, 4, 5, or more) different nucleic acids (e.g., two or more rAAV genomes, for example separately encapsidated by AAV capsid proteins), each encoding one or more different gene products. In some embodiments, two or more different compositions are administered to a subject, each composition comprising one or more nucleic acids encoding different gene products. In some embodiments, different gene products are operably linked to the same promoter type (e.g., the same promoter). In some embodiments, different gene products are operably linked to different promoters.

### Isolated Nucleic Acids and Vectors

An isolated nucleic acid may be DNA or RNA. The disclosure provides, in some aspects, an isolated nucleic acid comprising an expression construct encoding a Gcase (e.g., the gene product of GBA1 gene) or a portion thereof. Gcase, also referred to as β-glucocerebrosidase or GBA, refers to a lysosomal protein that cleaves the beta-glucosidic linkage of the chemical glucocerebroside, an intermediate in glycolipid metabolism. Deficiency in Gcase, a key lysosomal enzyme required for the normal metabolism of glycolipids, leads to the accumulation of the Gcase glycolipid substrates glucosylceramide (GluCer) and glucosylsphingosine (GluSph). In humans, Gcase is encoded by the GBA1 gene, located on chromosome 1. In some embodiments, GBA1 encodes a peptide that is represented by NCBI Reference Sequence NP_000148.2 (SEQ ID NO: 14). In some embodiments, the isolated nucleic acid comprises a Gcase-encoding sequence that has been codon optimized (e.g., codon optimized for expression in mammalian cells, for example human cells), such as the sequence set forth in SEQ ID NO: 15.

In some aspects, the disclosure provides an isolated nucleic acid comprising an expression construct encoding Prosaposin (e.g., the gene product of PSAP gene). Prosaposin is a precursor glycoprotein for sphingolipid activator proteins (saposins) A, B, C, and D, which facilitate the catabolism of glycosphingolipids with short oligosaccharide groups. In humans, the PSAP gene is located on chromosome 10. In some embodiments, PSAP encodes a peptide that is represented by NCBI Reference Sequence NP_002769.1 (e.g., SEQ ID NO: 16). In some embodiments, the isolated nucleic acid comprises a prosaposin-encoding sequence that has been codon optimized (e.g., codon optimized for expression in mammalian cells, for example human cells), such as the sequence set forth in SEQ ID NO: 17.

Aspects of the disclosure relate to an isolated nucleic acid comprising an expression construct encoding LIMP2/SCARB2 (e.g., the gene product of SCARB2 gene). SCARB2 refers to a membrane protein that regulates lysosomal and endosomal transport within a cell. In humans, SCARB2 gene is located on chromosome 4. In some embodiments, the SCARB2 gene encodes a peptide that is represented by NCBI Reference Sequence NP_005497.1 (SEQ ID NO: 18). In some embodiments, the isolated nucleic acid comprises the sequence set forth in SEQ ID NO: 19. In some embodiments the isolated nucleic acid comprises a SCARB2-encoding sequence that has been codon optimized.

In some aspects, the disclosure provides an isolated nucleic acid comprising an expression construct encoding a first gene product and a second gene product, wherein each gene product independently is selected from the gene products, or portions thereof, set forth in Table 1.

In some embodiments, an isolated nucleic acid or vector (e.g., rAAV vector) described by the disclosure comprises or consists of a sequence set forth in any one of SEQ ID NOs: 1-48. In some embodiments, an isolated nucleic acid or vector (e.g., rAAV vector) described by the disclosure comprises or consists of a sequence that is complementary (e.g., the complement of) a sequence set forth in any one of SEQ ID NOs: 1-48. In some embodiments, an isolated nucleic acid or vector (e.g., rAAV vector) described by the disclosure comprises or consists of a sequence that is a reverse complement of a sequence set forth in any one of SEQ ID NOs: 1-48. In some embodiments, an isolated nucleic acid or vector (e.g., rAAV vector) described by the disclosure comprises or consists of a portion of a sequence set forth in any one of SEQ ID NOs: 1-48. A portion may comprise at least 25%, 50%, 60%, 70%, 80%, 90%, 95%, or 99% of a sequence set forth in any one of SEQ ID NOs: 1-48. In some embodiments, a nucleic acid sequence described by the disclosure is a nucleic acid sense strand (e.g., 5′ to 3′ strand), or in the context of a viral sequences a plus (+) strand. In some embodiments, a nucleic acid sequence described by the disclosure is a nucleic acid antisense strand (e.g., 3′ to 5′ strand), or in the context of viral sequences a minus (−) strand.

In some embodiments, a gene product is encoded by a coding portion (e.g., a cDNA) of a naturally occurring gene. In some embodiments, a first gene product is a protein (or a fragment thereof) encoded by the GBA1 gene. In some embodiments, a gene product is a protein (or a fragment thereof) encoded by the SCARB2/LIMP2 gene and/or the PSAP gene. However, the skilled artisan recognizes that the order of expression of a first gene product (e.g., Gcase) and a second gene product (e.g., LIMP2) can generally be reversed (e.g., LIMP2 is the first gene product and Gcase is the second gene product). In some embodiments, a gene product is a fragment (e.g., portion) of a gene listed in Table 1. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1.

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornoe et al. (2002) Gene 297(1-2):21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34(Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7:2193. In some embodiments, the self-cleaving peptide is a T2A peptide.

Pathologically, disorders such as PD and Gaucher disease are associated with accumulation of protein aggregates composed largely of α-Synuclein (α-Syn) protein. Accordingly, in some embodiments, isolated nucleic acids described herein comprise an inhibitory nucleic acid that reduces or prevents expression of α-Syn protein. A sequence encoding an inhibitory nucleic acid may be placed in an untranslated region (e.g., intron, 5′UTR, 3′UTR, etc.) of the expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, shRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets SNCA (the gene encoding α-Syn protein). In some embodiments, the miRNA does not comprise any mismatches with the region of SNCA mRNA to which it hybridizes (e.g., the miRNA is “perfected”). In some embodiments, the inhibitory nucleic acid is an shRNA (e.g., an shRNA targeting SNCA). In some embodiments, an shRNA that targets SNCA is encoded by SEQ ID NO: 47. In some embodiments, an shRNA that targets SNCA is encoded by a sequence comprising SEQ ID NO: 20.

The skilled artisan recognizes that when referring to nucleic acid sequences comprising or encoding inhibitory nucleic acids (e.g., dsRNA, siRNA, shRNA, miRNA, amiRNA, etc.) any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, the vector is a recombinant AAV (rAAV) vector. In some embodiments, an rAAV vector is single-stranded (e.g., single-stranded DNA). In some embodiments, a vector is a Baculovirus vector (e.g., an Autographa cahfornica nuclear polyhedrosis (AcNPV) vector).

Typically an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26):2112-8.

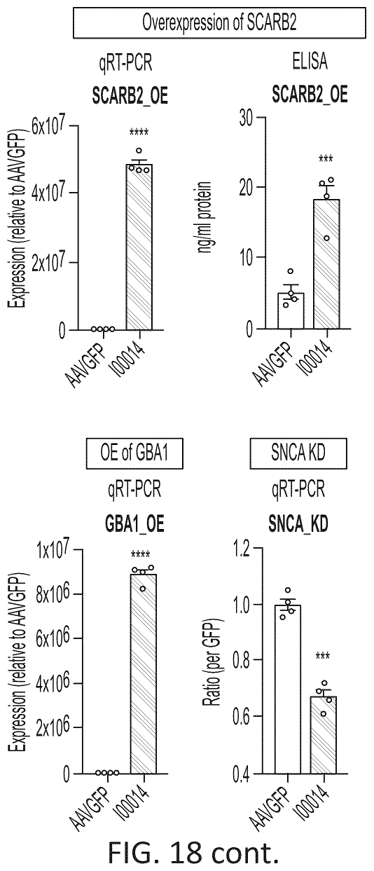

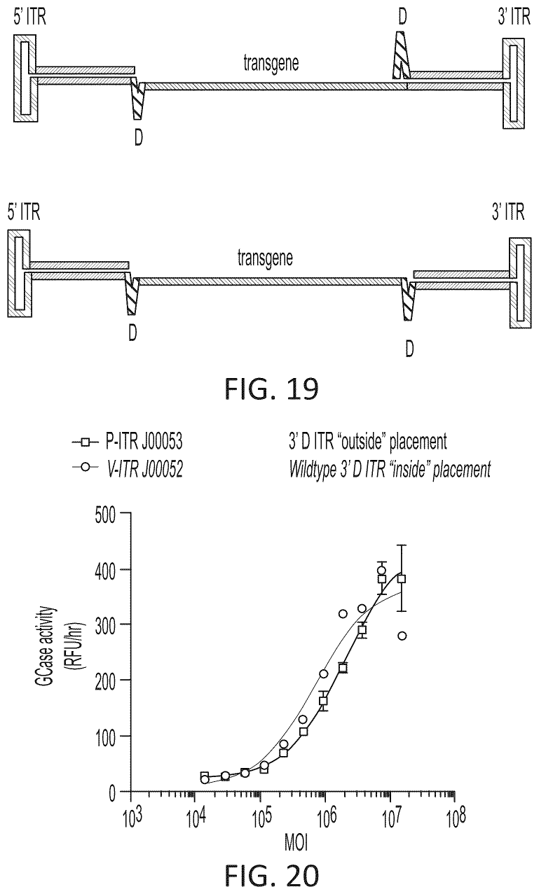

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 29). The structure of wild-type AAV2 ITR is shown in FIG. 19. Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region. (FIG. 19). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 27. The “D” region has been observed to play an important role in encapsidation of rAAV vectors by capsid proteins, for example as disclosed by Ling et al. (2015) J Mol Genet Med 9(3).

The disclosure is based, in part, on the surprising discovery that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs. In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 27). A modified “D” sequence may have at least 1, 2, 3, 4, 5, 6, 7, 8, 9, 10, or more than 10 nucleotide substitutions relative to a wild-type “D” sequence (e.g., SEQ ID NO: 27). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 27). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 27). In some embodiments, a modified “D” sequence comprises the sequence set forth in SEQ ID NO: 26, also referred to as an “S” sequence as described in Wang et al. (1995) J Mol Biol 250(5):573-80.

An isolated nucleic acid or rAAV vector as described by the disclosure may further comprise a “TRY” sequence, for example as set forth in SEQ ID NO: 28 or as described in Francois et al., (2005) J. Virol. 79(17):11082-11094. In some embodiments, a TRY sequence is positioned between an ITR (e.g., a 5′ ITR) and an expression construct (e.g., a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

### rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20, 25, 50, 100, etc.) amino acid substitutions (e.g., mutations) relative to the wild-type AAV capsid protein from which it is derived. In some embodiments, an AAV capsid protein variant is an AAV1RX capsid protein, for example as described by Albright et al. Mol Ther. 2018 Feb. 7; 26(2):510-523. In some embodiments, a capsid protein variant is an AAV TM6 capsid protein, for example as described by Rosario et al. Mol Ther Methods Clin Dev. 2016; 3:16026.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63(9):3822-8 and Wright (2009) Hum Gene Ther. 20(7):698-706. In some embodiments, an rAAV comprises a capsid protein that specifically or preferentially targets myeloid cells, for example microglial cells.

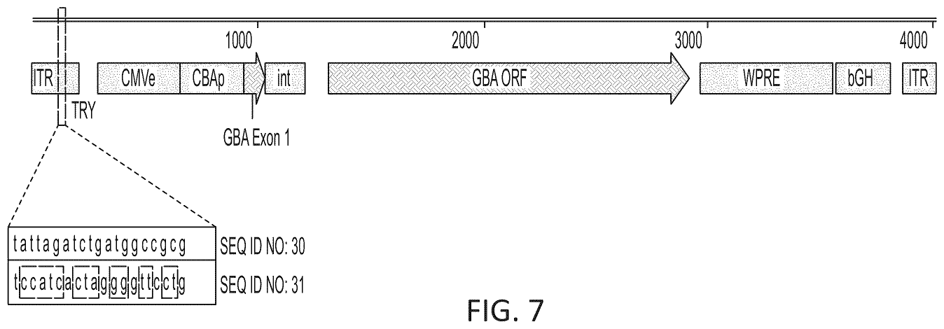

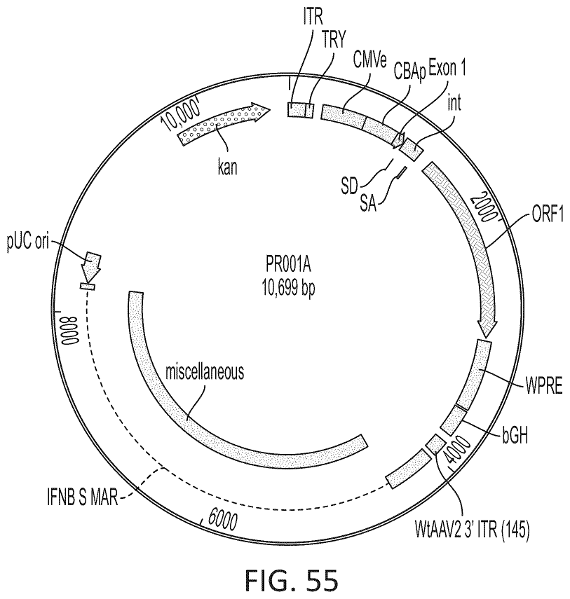

In some embodiments, the disclosure provides an rAAV referred to as “PR001”. This rAAV expresses the codon-optimized coding sequence of human GBA1 (SEQ ID NO:15). In some embodiments, the disclosure provides an rAAV referred to as “PR001A”. PR001A (AAV9.CBA.GBA1.A) is a rAAV that delivers a functional human GBA1 gene, leading to increased expression of functional human Gcase. The PR001A vector insert comprises the chicken β-actin (CBA) promoter element, comprising 4 parts: the cytomegalovirus (CMV) enhancer, CBA promoter, exon 1, and intron (int) to constitutively express the codon-optimized coding sequence of human GBA1 (SEQ ID NO:15). The 3′ region also contains a woodchuck hepatitis virus post-transcriptional regulatory element (WPRE) followed by a bovine growth hormone polyadenylation signal tail. Three well described transcriptional regulatory activation sites are included at the 5′ end of the promoter region: TATA, RBS, and YY1 (see, e.g., Francois et al., (2005) J. Virol. 79(17):11082-11094). The flanking inverted terminal repeats (ITRs) allow for the correct packaging of the intervening sequences. Two variants of the 5′ ITR sequence (FIG. 7, inset box, bottom sequence) are provided; these variants have several nucleotide differences within the 20-nucleotide “D” region of the ITR, which is believed to impact the efficiency of packaging and expression. PR001A contains the “D” domain nucleotide sequence shown in FIG. 7 (inset box, top sequence; SEQ ID NO:30). In some embodiment, the disclosure provides a variant vector referred to as “PR001B”, which harbors a mutant “D” domain (termed an “S” domain herein, with the nucleotide changes shown by shading in SEQ ID NO:31 in FIG. 7). Except for the different 5′ITR sequence, PR001B is identical to PR001A. The backbone contains the gene to confer resistance to kanamycin as well as a stuffer sequence to prevent reverse packaging. A schematic depicting a plasmid encoding the rAAV vector is shown in FIG. 55. SEQ ID NO: 39 provides the nucleotide sequence of the first strand (in 5′ to 3′ order) of the plasmid encoding the PR001A vector shown in FIG. 55. SEQ ID NO: 40 provides the nucleotide sequence of the second strand (in 5′ to 3′ order) of the plasmid encoding PR001A vector shown in FIG. 55. PR001A comprises AAV9 capsid proteins.

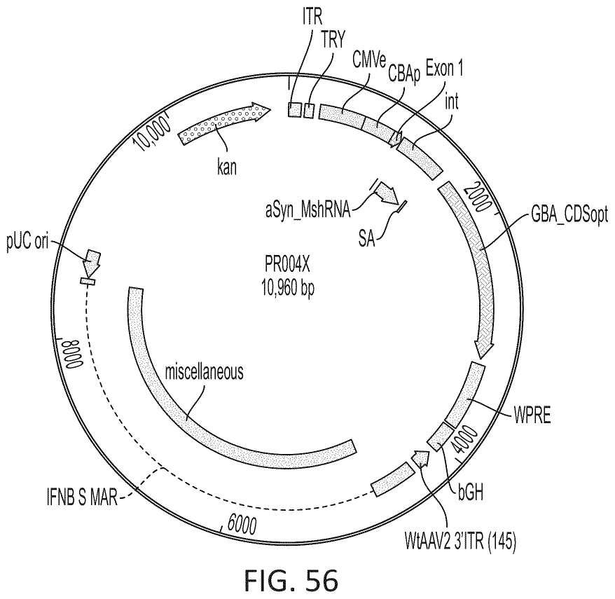

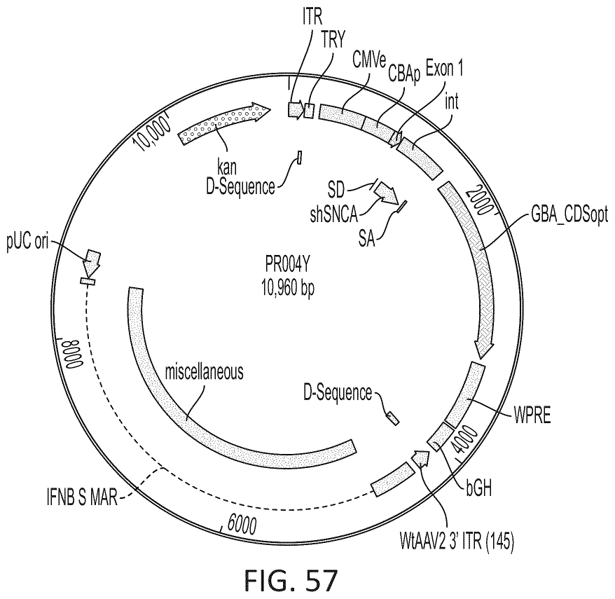

In some embodiments, the disclosure provides an rAAV referred to as “PR004”. This rAAV expresses the codon-optimized coding sequence of human GBA1 (SEQ ID NO:15) and an inhibitory nucleic acid coding sequence that targets reduces α-Synuclein and comprises the nucleotide sequence of SEQ ID NO: 20. In some embodiments, the disclosure provides an rAAV referred to as “PR004X”. In some embodiments, the disclosure provides an rAAV referred to as “PR004Y”. Each of PR004X and PR004Y is a rAAV that (i) delivers a functional human GBA1 gene, leading to increased expression of functional human Gcase, and (ii) encodes a shRNA that reduces α-Synuclein levels via RNA interference. The PR004 vector insert comprises the chicken β-actin (CBA) promoter element, comprising 4 parts: the cytomegalovirus (CMV) enhancer, CBA promoter, exon 1, and intron (int) to constitutively express the codon-optimized coding sequence of human GBA1 (SEQ ID NO:15) and an inhibitory nucleic acid coding sequence comprising the nucleotide sequence of SEQ ID NO: 20. The 3′ region also contains a woodchuck hepatitis virus post-transcriptional regulatory element (WPRE) followed by a bovine growth hormone polyadenylation signal tail. Three well described transcriptional regulatory activation sites are included at the 5′ end of the promoter region: TATA, RBS, and YY1 (see, e.g., Francois et al., (2005) J. Virol. 79(17):11082-11094). The flanking inverted terminal repeats (ITRs) allow for the correct packaging of the intervening sequences. The backbone contains the gene to confer resistance to kanamycin as well as a stuffer sequence to prevent reverse packaging. A schematic depicting a plasmid encoding the rAAV PR004X vector is shown in FIG. 56. SEQ ID NO: 41 provides the nucleotide sequence of the first strand (in 5′ to 3′ order) of the plasmid encoding the PR004X vector shown in FIG. 56. SEQ ID NO: 42 provides the nucleotide sequence of the second strand (in 5′ to 3′ order) of the plasmid encoding the PR004X vector shown in FIG. 56. A schematic depicting a plasmid encoding the rAAV PR004Y vector is shown in FIG. 57. SEQ ID NO: 43 provides the nucleotide sequence of the first strand (in 5′ to 3′ order) of the plasmid encoding the PR004Y vector shown in FIG. 57. SEQ ID NO: 44 provides the nucleotide sequence of the second strand (in 5′ to 3′ order) of the plasmid encoding the PR004Y vector shown in FIG. 57. PR004X and PR004Y each comprise AAV9 capsid proteins. The PR004X and PR004Y vectors are designed to reduce accumulation of all forms of α-Synuclein, including aggregated and extracellular forms.

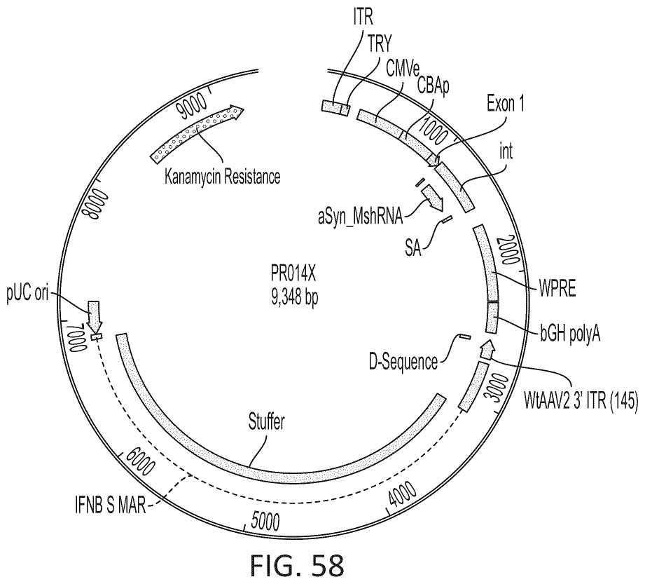

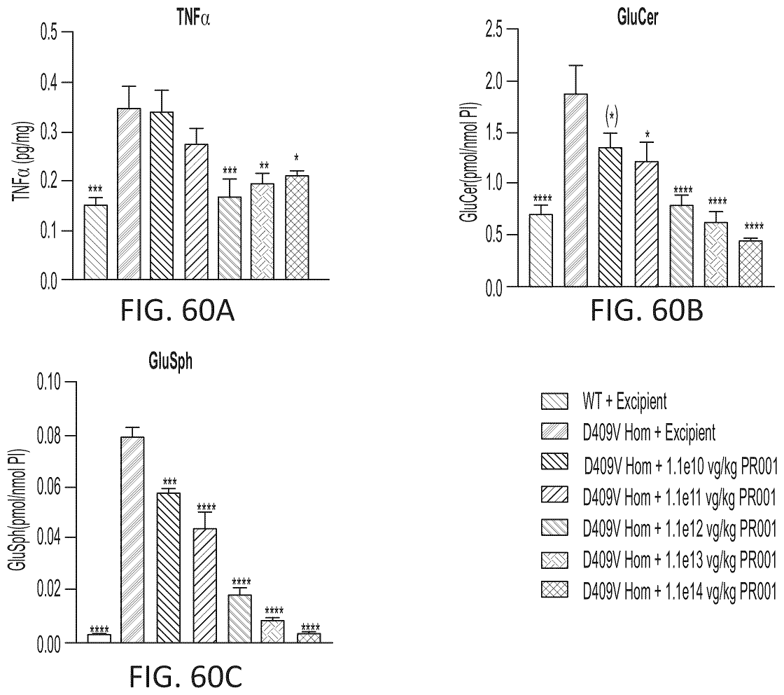

In some embodiments, the disclosure provides an rAAV referred to as “PR014”. This rAAV expresses an inhibitory nucleic acid coding sequence that targets reduces α-Synuclein and comprises the nucleotide sequence of SEQ ID NO: 20. In some embodiments, the disclosure provides an rAAV referred to as “PR014X”. PR014X is a rAAV that encodes a shRNA that reduces α-Synuclein levels via RNA interference. The PR014X vector insert comprises the chicken β-actin (CBA) promoter element, comprising 4 parts: the cytomegalovirus (CMV) enhancer, CBA promoter, exon 1, and intron (int) to constitutively express an inhibitory nucleic acid coding sequence comprising the nucleotide sequence of SEQ ID NO: 20. The 3′ region also contains a woodchuck hepatitis virus post-transcriptional regulatory element (WPRE) followed by a bovine growth hormone polyadenylation signal tail. Three well described transcriptional regulatory activation sites are included at the 5′ end of the promoter region: TATA, RBS, and YY1 (see, e.g., Francois et al., (2005) J. Virol. 79(17):11082-11094). The flanking inverted terminal repeats (ITRs) allow for the correct packaging of the intervening sequences. The backbone contains the gene to confer resistance to kanamycin as well as a stuffer sequence to prevent reverse packaging. A schematic depicting a plasmid encoding the rAAV vector is shown in FIG. 58. SEQ ID NO: 45 provides the nucleotide sequence of the first strand (in 5′ to 3′ order) of the plasmid encoding the PR014X vector shown in FIG. 60. SEQ ID NO: 46 provides the nucleotide sequence of the second strand (in 5′ to 3′ order) of the plasmid encoding the PR014X vector shown in FIG. 60. SEQ ID NO: 47 provides the nucleotide sequence of the first strand (in 5′ to 3′ order) of the shRNA in the plasmid encoding the PR014X vector shown in FIG. 58. SEQ ID NO: 48 provides the nucleotide sequence of the second strand (in 5′ to 3′ order) of the shRNA in the plasmid encoding the PR014X vector shown in FIG. 58. PR014X comprises AAV9 capsid proteins. The PR014X vector is designed to reduce accumulation of all forms of α-Synuclein, including aggregated and extracellular forms.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes). In some embodiments, an rAAV as disclosed herein is produced in HEK293 (human embryonic kidney) cells.

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

In some embodiments, the disclosure provides a PR001 (e.g., PR001A) finished drug product comprising the PR001 rAAV described above presented in aqueous solution. In some embodiments, the final formulation buffer comprises about 20 mM Tris [pH 8.0], about 1 mM MgCl₂, about 200 mM NaCl, and about 0.001% [w/v] poloxamer 188. In some embodiments, the finished drug product and the final formulation buffer are suitable for intra-cisterna magna (ICM) injection or intravenous administration.

### Methods

Aspects of the disclosure relate to delivery of compositions (e.g., isolated nucleic acids, rAAVs, etc.) engineered to express CNS disease-associated gene products to a cell or cells (e.g., a cell or cells of a subject).

As described further in the Examples section, aspects of the disclosure relate to compositions expressing gene products that inhibit or prevent glial scarring (e.g., gliosis). Accordingly, in some aspects, the disclosure provides a method for inhibiting glial scarring in a subject, the method comprising administering to the subject a composition (e.g., an isolated nucleic acid or rAAV) as described herein.

In some embodiments, the subject has or is suspected of having a central nervous system (CNS) disease. In some embodiments, the subject has Gaucher disease (GD). In some embodiments, the subject has neuronopathic GD (nGD) (e.g., Type 2 GD or Type 3 GD). In some embodiments, the subject has Type 1 GD. In some embodiments, a subject having GD does not have PD or PD symptoms. In some embodiments, the subject has parkinsonism. In some embodiments, a subject has Parkinson's disease (PD). In some embodiments, the subject has an atypical Parkinsonian disorder. In some embodiments, an atypical Parkinsonian disorder is dementia with Lewy bodies, progressive supranuclear palsy, multiple system atrophy or corticobasal syndrome.

The disclosure is based, in part, on compositions for expression of one or more CNS disease-associated gene products in a subject to treat CNS-associated diseases. The one or more CNS disease-associated gene products may be encoded by one or more isolated nucleic acids or rAAV vectors. In some embodiments, a subject is administered a single vector (e.g., isolated nucleic acid, rAAV, etc.) encoding one or more (1, 2, 3, 4, 5, or more) gene products. In some embodiments, a subject is administered a plurality (e.g., 2, 3, 4, 5, or more) vectors (e.g., isolated nucleic acids, rAAVs, etc.), where each vector encodes a different CNS disease-associated gene product. In some embodiments, the composition expresses GBA or a portion thereof. In some embodiments, the composition expresses an interfering RNA that targets alpha-Synuclein. In some embodiments, the composition expresses GBA or a portion thereof and an interfering RNA that targets alpha-Synuclein.

A CNS-associated disease may be a neurodegenerative disease, synucleinopathy, tauopathy, or a lysosomal storage disease. Examples of neurodegenerative diseases and their associated genes are listed in Table 4.

A “synucleinopathy” refers to a disease or disorder characterized by the accumulation of alpha-Synuclein (the gene product of SNCA) in a subject (e.g., relative to a healthy subject, for example a subject not having a synucleinopathy). Examples of synucleinopathies and their associated genes are listed in Table 5.

A “tauopathy” refers to a disease or disorder characterized by accumulation of abnormal Tau protein in a subject (e.g., relative to a healthy subject not having a tauopathy). .Examples of tauopathies and their associated genes are listed in Table 6.

A “lysosomal storage disease” refers to a disease characterized by abnormal build-up of toxic cellular products in lysosomes of a subject. Examples of lysosomal storage diseases and their associated genes are listed in Table 7.

As used herein “treat” or “treating” refers to (a) preventing or delaying onset of a CNS disease; (b) reducing severity of a CNS disease; (c) reducing or preventing development of symptoms characteristic of a CNS disease; (d) and/or preventing worsening of symptoms characteristic of a CNS disease. Symptoms of CNS disease may include, for example, motor dysfunction (e.g., shaking, rigidity, slowness of movement, difficulty with walking, paralysis), cognitive dysfunction (e.g., dementia, depression, anxiety, psychosis), difficulty with memory, and emotional and behavioral dysfunction.

The disclosure is based, in part, on compositions for expression of one or more PD-associated gene products in a subject that act together (e.g., synergistically) to treat Parkinson's disease.

Accordingly, in some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

The disclosure is based, in part, on compositions for expression of one or more CNS disease-associated gene products in a subject to treat Gaucher disease (GD). The diagnosis of GD is established by the presence of biallelic pathogenic mutations in GBA1 or a finding of less than 15% of normal GCase activity in peripheral blood leukocytes. GBA1 mutations causing more profound enzyme deficiencies are associated with earlier onset of disease, faster progression of symptoms, and a higher likelihood to develop neurological symptoms (Svennerholm et al., Clin Genet. 1986;30(2):131-5; Cox, Biologics. 2010;4:299-313). GD has traditionally been subdivided into three broader phenotypes distinguished by the presence of neurologic manifestations (neuronopathic [Type 2 GD and Type 3 GD; nGD] or non-neuronopathic [Type 1 GD]).

Within nGD, the distinctions between Type 2 GD and Type 3 GD may represent a phenotypic continuum of an acute to chronic presentation of CNS and visceral symptoms. Infants with Type 2 GD, known as the acute neuronopathic form, classically present with early bulbar signs (such as squint and/or swallowing difficulty), opisthotonus or spasticity, supranuclear gaze palsy, and failure to achieve motor, behavior, and cognitive milestones. Most children die by age 2. (Goker-Alpan et al., J Pediatr. 2003;143(2):273-6; Roshan and Sidransky, Diseases. 2017;5(1):pii:E10). In Type 3 GD, the hallmark clinical sign is a slow horizontal supranuclear gaze palsy, with other neurologic manifestations ranging from cognitive impairment to ataxia to seizures to death in childhood or early adolescence (Goker-Alpan et al., J Pediatr. 2003;143(2):273-6; Tylki-Szymańska et al., J Inherit Metab Dis. 2010;33(4):339-46).

Accordingly, in some aspects, the disclosure provides a method for treating a subject having or suspected of having neuronopathic Gaucher disease, the method comprising administering to the subject a composition (e.g., a composition comprising an isolated nucleic acid or a vector or a rAAV) as described by the disclosure.

In some aspects, the disclosure provides a method for treating a subject having Type 2 Gaucher disease or Type 3 Gaucher disease, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the disclosure provides a method for treating a neurological symptom of a subject having Type 2 Gaucher disease or Type 3 Gaucher disease, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, a neurological symptom of Type 2 Gaucher disease or Type 3 Gaucher disease is supranuclear gaze palsy, hypotonia, seizures, spasticity, hypokinesia, motor or behavioral developmental delay or impairment, cognitive delay or impairment, ataxia, intention tremor, or rigidity.

In some embodiments, patients having certain forms of Gaucher disease exhibit symptoms of peripheral neuropathy, for example as described in Biegstraaten et al. (2010) Brain 133(10):2909-2919. In some embodiments, the disclosure provides a method for treating peripheral neuropathy in a subject having Gaucher disease (e.g., Type 1 Gaucher disease), the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the disclosure provides a method for treating Type 1 Gaucher disease in a subject, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the rAAV is administered to the subject intravenously for treating Type 1 Gaucher disease.

In some embodiments, the disclosure provides a method for treating a subject having Parkinson's disease (PD) with a glucocerebrosidase-1 (GBA1) mutation (e.g., a pathogenic GBA1 mutation), the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, the disclosure provides a method for treating a symptom of a subject having PD with a GBA1 mutation, the method comprising administering to the subject a rAAV comprising a nucleic acid comprising an expression construct comprising a promoter operably linked to a sequence encoding a Gcase protein, wherein the sequence encoding a Gcase protein comprises SEQ ID NO:15; and wherein the rAAV comprises a capsid protein having an AAV9 serotype. In some embodiments, a motor symptom of PD is resting tremor, bradykinesia, rigidity, or gait difficulty. In some embodiments, a non-motor symptom of PD is cognitive impairment/dementia, depression, delusions/hallucinations, psychosis, sleep disturbances, constipation, urinary symptoms, pain, anosmia, difficulty swallowing, or hypotension. In some embodiments, the subject having PD has one GBA1 mutation. In some embodiments, the subject having PD has two GBA1 mutations.

In some embodiments, a rAAV encoding a Gcase protein for treating Type 1 Gaucher disease, Type 2 Gaucher disease or Type 3 Gaucher disease or Parkinson's disease with a GBA1 mutation is administered to a subject at a dose ranging from about 1×10¹² vector genomes (vg) to about 1×10¹⁵ vg, or from about 1×10¹³ vg to about 5×10¹⁴ vg, or from about 5×10¹³ vg to about 5×10¹⁴ vg, or from about 3.4×10¹³ vg to about 1×10¹⁴ vg, or from about 1×10¹⁴ vg to about 5×10¹⁴ vg, or from about 1×10¹⁴ vg to about 3×10¹⁴ vg, or from about 1×10¹⁴ vg to about 2×10¹⁴ vg. The total dose assumes an adult brain mass of 1.3 kg (Hakim and Mathieson, Neurology, 1979;29(9 Pt 1):1209-14). For pediatric subjects, the dose may be scaled accordingly. In some embodiments, the dose for pediatric subjects may be adjusted using estimates of brain weight by age, for example, based on a composite dataset that includes derived brain weights from 21 autopsy and neuroimaging publications (Vannucci and Vannucci, Am J Phys Anthropol. 2019;168(2):247-61).

In some embodiments, a rAAV encoding a Gcase protein for treating Parkinson's disease with a GBA1 mutation is administered to a subject (e.g., a human adult subject) at a dose of about 1×10¹⁴ vg, about 2×10¹⁴ vg, about 3×10¹⁴ vg, about 4×10¹⁴ vg, or about 5×10¹⁴ vg. In some embodiments, a rAAV for treating Parkinson's disease with a GBAJ mutation is administered to a subject (e.g., a human adult subject) at a dose of about 1×10¹⁴ vg (about 7.7×10¹⁰ vg/g brain), about 2×10¹⁴ vg (about 1.5×10¹¹ vg/g brain), or about 3×10¹⁴ vg (about 1.9×10¹¹ vg/g brain).

In some embodiments, a rAAV encoding a Gcase protein for treating Type 2 or Type 3 Gaucher disease is administered to a subject (e.g., a human pediatric subject) at a dose ranging from about 5×10¹⁰ vg/g brain to about 5×10¹¹ vg/g brain. In some embodiments, a rAAV for treating Type 2 Gaucher disease or Type 3 Gaucher disease is administered to a subject (e.g., a human pediatric subject) at a dose of about 1.3×10¹¹ vg/g brain (from about 5.9×10¹³ vg to about 1.7×10¹⁴ vg). In some embodiments, a rAAV for treating Type 2 Gaucher disease or Type 3 Gaucher disease is administered to a subject (e.g., a human pediatric subject) at a dose of about 1.9×10¹¹ vg/g brain (from about 8.6×10¹³ vg to about 2.5×10¹⁴ vg). In some embodiments, a rAAV for treating Type 2 Gaucher disease or Type 3 Gaucher disease is administered to a subject (e.g., a human pediatric subject) at a dose of about 7.7×10¹⁰ vg/g brain (from about 3.4×10¹³ vg to about 1×10¹⁴ vg) or a dose of about 2.3×10¹¹ vg/g brain (from about 1×10¹⁴ vg to about 3×10¹⁴ vg).

In some embodiments, a rAAV encoding a Gcase protein for treating Type 1, Type 2 or Type 3 Gaucher disease or Parkinson's disease with a GBA1 mutation is administered to a subject as a single dose, and the rAAV is not administered to the subject subsequently.

In some embodiments, a rAAV encoding a Gcase protein is administered via a single suboccipital injection into the cisterna magna. In some embodiments, the injection into the cisterna magna is performed under radiographic guidance.

In some embodiments, the disclosure provides a method for treating a subject having a synucleinopathy or parkinsonism, the method comprising administering to the subject a rAAV comprising a nucleic acid comprising an expression construct comprising a transgene comprising (a) a Gcase protein coding sequence comprising the nucleotide sequence of SEQ ID NO: 15; and (b) an inhibitory nucleic acid coding sequence comprising the nucleotide sequence of SEQ ID NO: 20 or SEQ ID NO: 47; wherein the rAAV comprises a capsid protein having an AAV9 serotype.

In some embodiments, the disclosure provides a method for treating a subject having multiple system atrophy, Parkinson's disease, Parkinson's disease with GBA1 mutation, Lewy body disease, dementia with Lewy bodies, dementia with Lewy bodies with GBA1 mutation, progressive supranuclear palsy, or corticobasal syndrome, the method comprising administering to the subject a rAAV comprising a nucleic acid comprising an expression construct comprising a transgene comprising (a) a Gcase protein coding sequence comprising the nucleotide sequence of SEQ ID NO: 15; and (b) an inhibitory nucleic acid coding sequence comprising the nucleotide sequence of SEQ ID NO: 20 or SEQ ID NO: 47; wherein the rAAV comprises a capsid protein having an AAV9 serotype.

In some embodiments, the disclosure provides a method for treating a subject having a synucleinopathy or parkinsonism, the method comprising administering to the subject a rAAV comprising a nucleic acid comprising an expression construct comprising a transgene comprising an inhibitory nucleic acid coding sequence comprising the nucleotide sequence of SEQ ID NO: 20 or SEQ ID NO: 47; wherein the rAAV comprises a capsid protein having an AAV9 serotype.

In some embodiments, the disclosure provides a method for treating a subject having multiple system atrophy, Parkinson's disease, Parkinson's disease with GBA1 mutation, Lewy body disease, dementia with Lewy bodies, dementia with Lewy bodies with GBA1 mutation, progressive supranuclear palsy, or corticobasal syndrome, the method comprising administering to the subject a rAAV comprising a nucleic acid comprising an expression construct comprising a transgene comprising an inhibitory nucleic acid coding sequence comprising the nucleotide sequence of SEQ ID NO: 20 or SEQ ID NO: 47; wherein the rAAV comprises a capsid protein having an AAV9 serotype.

A subject is typically a mammal, preferably a human. In some embodiments, a subject is between the ages of 1 month old and 10 years old (e.g., 1 month, 2 months, 3 months, 4, months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3, years, 4 years, 5 years, 6 years, 7 years, 8 years, 9 years, 10 years, or any age therebetween). In some embodiments, a subject is between 2 years old and 20 years old. In some embodiments, a subject is between 30 years old and 100 years old. In some embodiments, a subject is older than 55 years old.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, a composition is administered to a subject by intra-cisterna magna (ICM) injection. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

In some embodiments, a subject is administered an immunosuppressant prior to (e.g., between 1 month and 1 minute prior to) or at the same time as a composition as described herein. In some embodiments, the immunosuppressant is a corticosteroid (e.g., prednisone, budesonide, etc.), an mTOR inhibitor (e.g., sirolimus, everolimus, etc.), an antibody (e.g., adalimumab, etanercept, natalizumab, etc.), or methotrexate.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10 ¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection. In some embodiments, a rAAV as described herein is administered to a subject at a dose ranging from about 1×10¹⁰ vector genomes (vg) to about 1×10¹⁷ vg by intravenous injection. In some embodiments, a rAAV as described herein is administered to a subject at a dose ranging from about 1×10¹⁰ vg to about 1×10¹⁶ vg by injection into the cisterna magna.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Example 1: rAAV Vectors

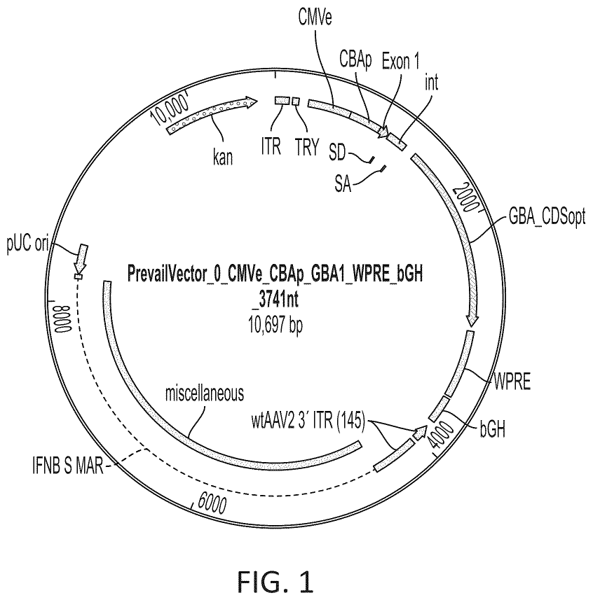

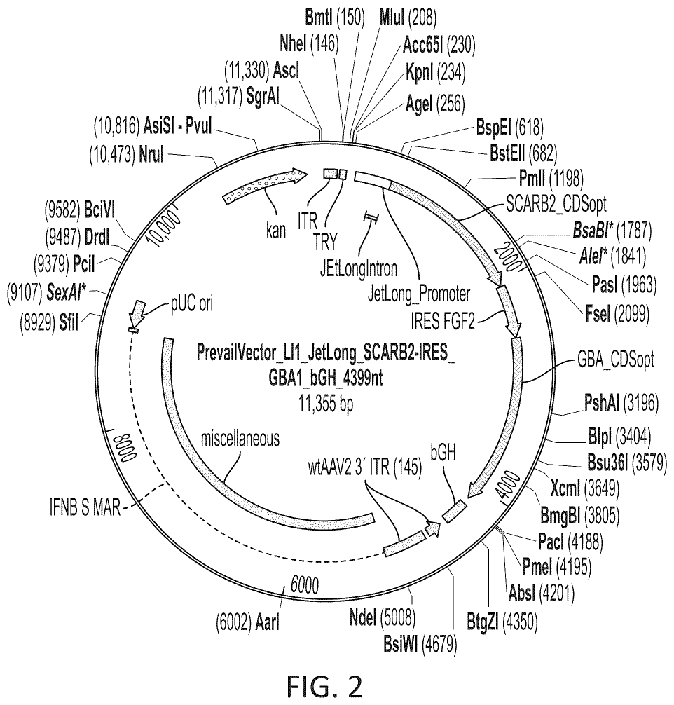

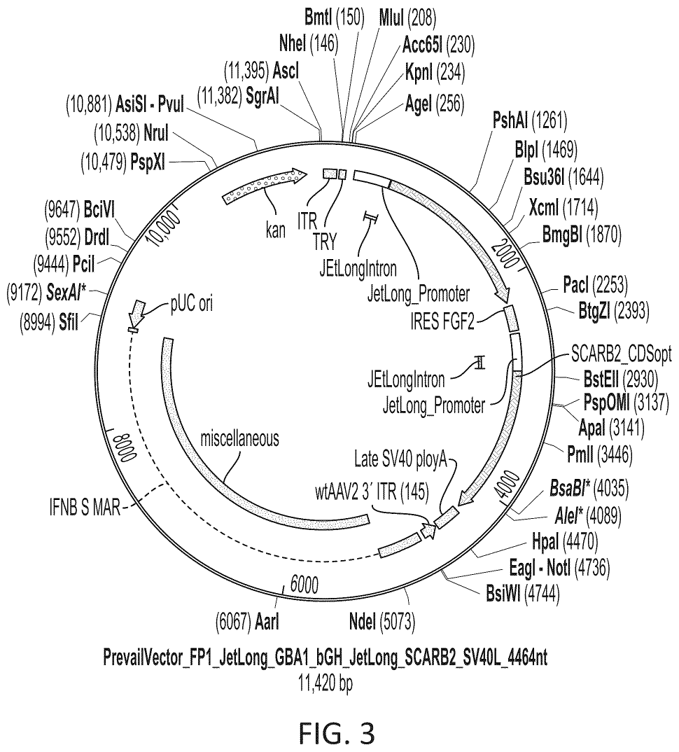

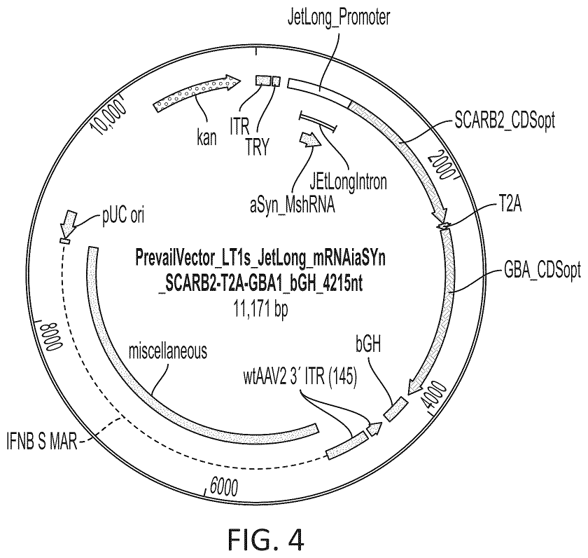

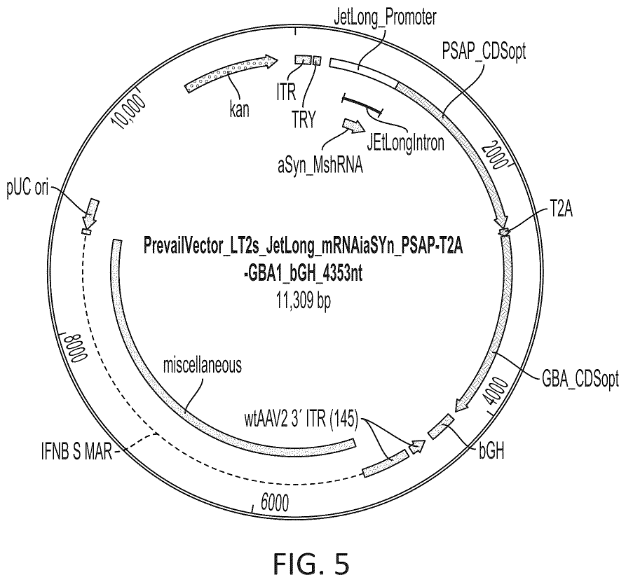

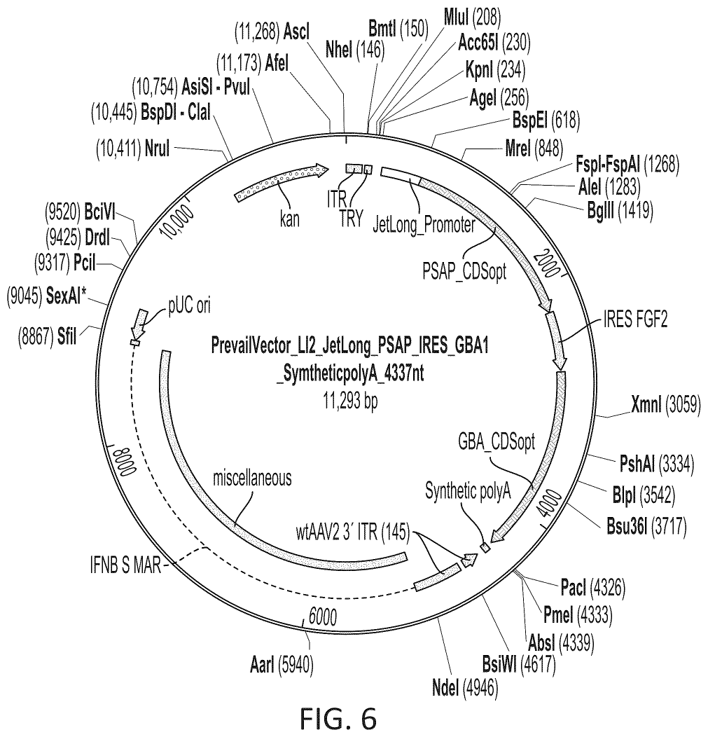

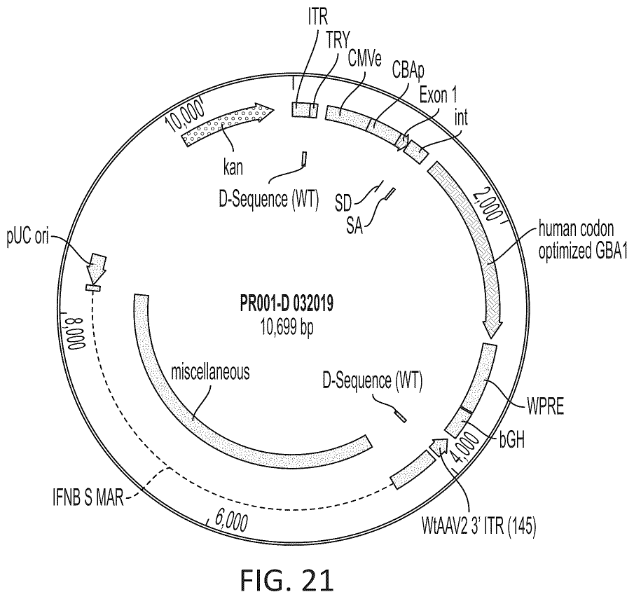

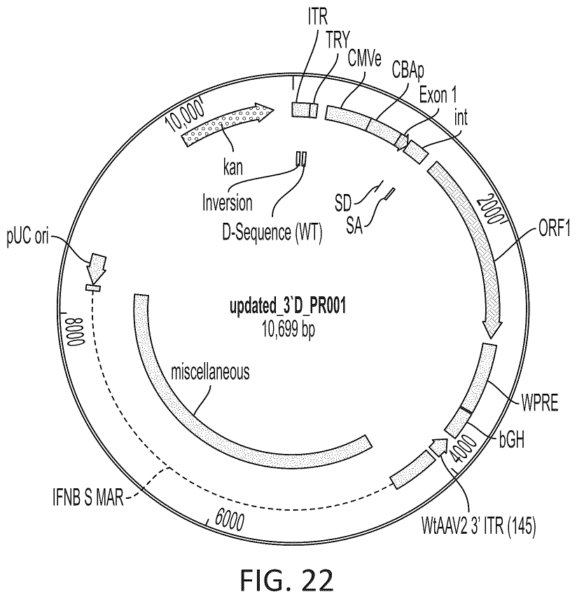

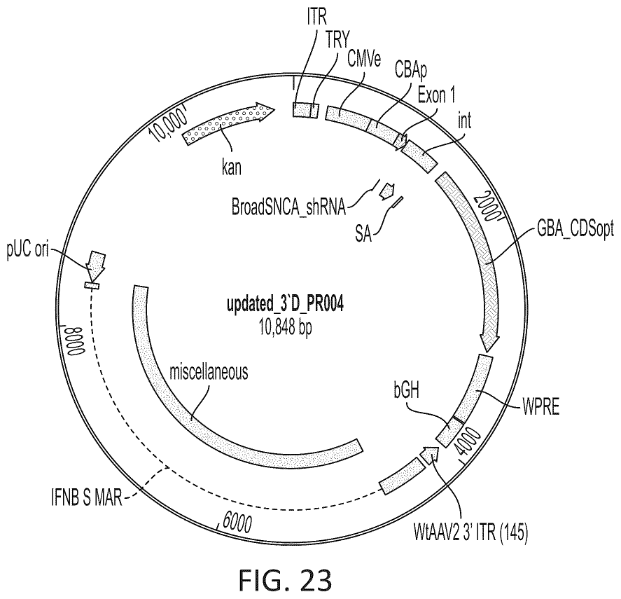

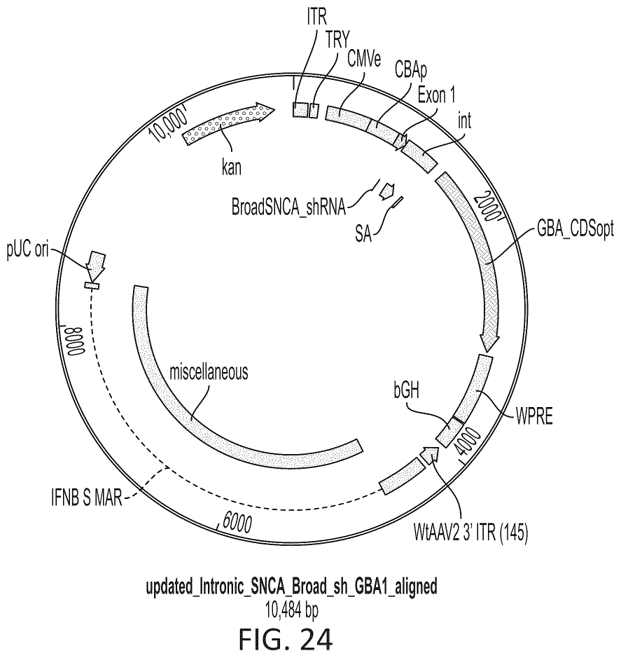

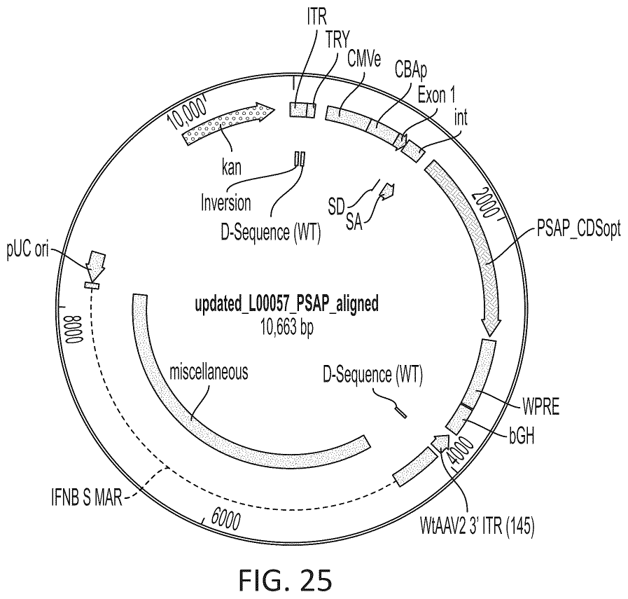

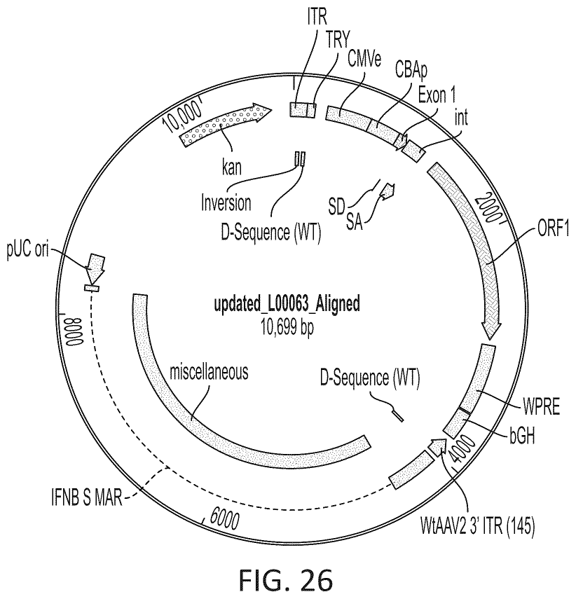

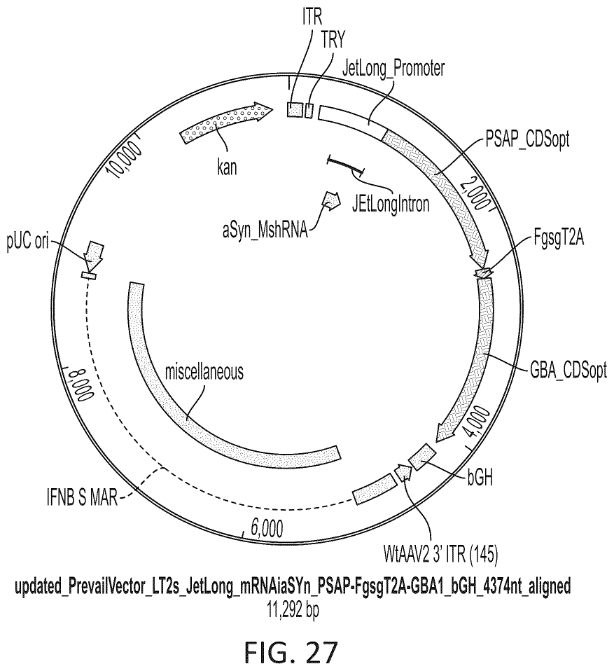

AAV vectors are generated using cells, such as HEK293 cells for triple-plasmid transfection. The ITR sequences flank an expression construct comprising a promoter/enhancer element for each transgene of interest, a 3′ polyA signal, and posttranslational signals such as the WPRE element. Multiple gene products can be expressed simultaneously such as GBA1 and LIMP2 and/or Prosaposin, by fusion of the protein sequences; or using a 2A peptide linker, such as T2A or P2A, which leads 2 peptide fragments with added amino acids due to prevention of the creation of a peptide bond; or using an IRES element; or by expression with 2 separate expression cassettes. The presence of a short intronic sequence that is efficiently spliced, upstream of the expressed gene, can improve expression levels. shRNAs and other regulatory RNAs can potentially be included within these sequences. Examples of plasmids comprising rAAV vectors described by the disclosure are shown in FIGS. 1-6, FIGS. 21-27, and FIGS. 55-58 and in Table 2 below.

TABLE 2

| | Pro- | | | | Bicis- | Pro- | | | Length |
|---|---|---|---|---|---|---|---|---|---|
| | moter | | | PolyA | tronic | moter | | | between |
| Name | 1 | shRNA | CDS1 | 1 | element | 2 | CDS2 | PolyA2 | ITRs |
| CMVe_CBAp_GBA1_WPRE_bGH | CBA | | GBA1 | WPRE- | | | | | 3741 |
| | | | | bGH | | | | | |
| LT1s_JetLong_mRNAiaSYn_SCARB2-T2A-GBA1_bGH | JetLong | aSyn | SCARB2 | bGH | T2A | | GBA1 | | 4215 |
| LI1_JetLong_SCARB2-IRES-GBA1_bGH | JetLong | | SCARB2 | bGH | IRES | | GBA1 | | 4399 |
| FP1_JetLong_GBA1_bGH_JetLong_SCARB2_SV40L | JetLong | | GBA1 | bGH | | JetLong | SCARB2 | SV40L | 4464 |
| PrevailVector_LT2s_JetLong_mRNAiaSYn_PSAP- | JetLong | aSyn | PSAP | bGH | T2A | — | GBA1 | — | 4353 |
| T2A-GBA1_bGH_4353nt | | | | | | | | | |
| PrevailVector_LI2_JetLong_PSAP_IRES_GBA1_ | JetLong | — | PSAP | Synthetic | IRES | — | GBA1 | — | 4337 |
| SymtheticpolyA_4337nt | | | | pA | | | | | |

### Example 2: Cell Based Assays of Viral Transduction into GBA-Deficient Cells

Cells deficient in GBA1 are obtained, for example as fibroblasts from GD patients, monocytes, or hES cells, or patient-derived induced pluripotent stem cells (iPSCs). These cells accumulate substrates such as glucosylceramide and glucosylsphingosine (GluCer and GluSph). Treatment of wild-type or mutant cultured cell lines with Gcase inhibitors, such as CBE, is also be used to obtain GBA deficient cells.

Using such cell models, lysosomal defects are quantified in terms of accumulation of protein aggregates, such as of α-Synuclein with an antibody for this protein or phospho-aSyn, followed by imaging using fluorescent microscopy. Imaging for lysosomal abnormalities by ICC for protein markers such as LAMP1, LAMP2, LIMP 1, LIMP2, or using dyes such as Lysotracker, or by uptake through the endocytic compartment of fluorescent dextran or other markers is also performed. Imaging for autophagy marker accumulation due to defective fusion with the lysosome, such as for LC3, can also be performed. Western blotting and/or ELISA is used to quantify abnormal accumulation of these markers. Also, the accumulation of glycolipid substrates and products of GBA1 is measured using standard approaches.

Therapeutic endpoints (e.g., reduction of PD-associated pathology) are measured in the context of expression of transduction of the AAV vectors, to confirm and quantify activity and function. Gcase can also be quantified using protein ELISA measures, or by standard Gcase activity assays.

### Example 2.1: In Vitro Pharmacology Studies with rAAV Encoding Gcase

### Transduction and Potency

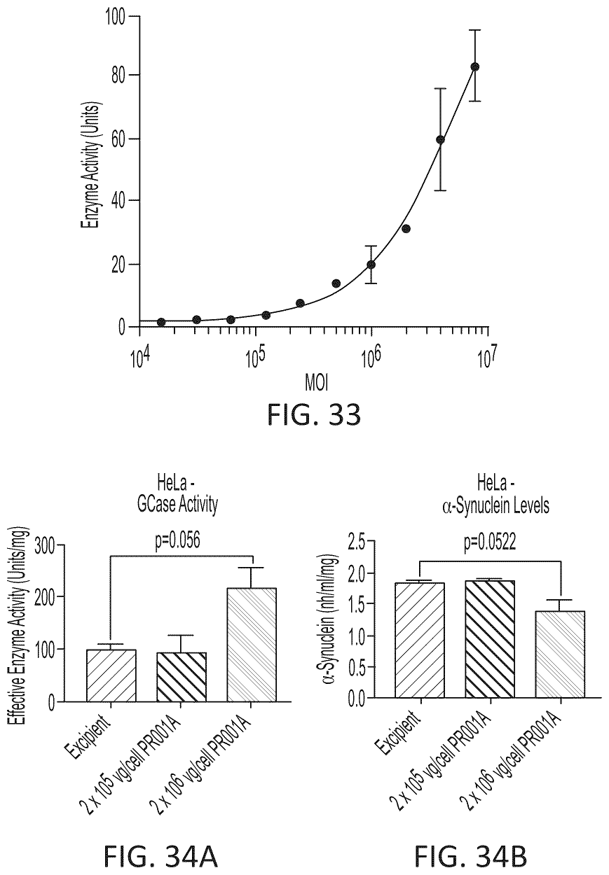

An in vitro study to evaluate the ability of PR001A (AAV9.CBA.GBA1.A) (schematic of a plasmid encoding the vector provided in FIG. 55), comprising the codon-optimized coding sequence of human GBA1 (SEQ ID NO:15), to express the GBA1 transgene in HEK293T cells demonstrated a dose-dependent increase in GCase activity following PR001A transduction in HEK293T cells (FIG. 33).

### Efficacy Measures (α-Synuclein)

In vitro studies were also conducted in HeLa cells, a human cell line, and in primary mouse hippocampal neurons. In HeLa cells treated with 2×10⁶ vg/cell PR001A, an approximately 2-fold increase in GCase activity levels and a reduction in total α-Synuclein levels compared to excipient-treated control cells was observed (FIG. 34A and FIG. 34B). A similar effect was not observed with a lower dose of PR001A.

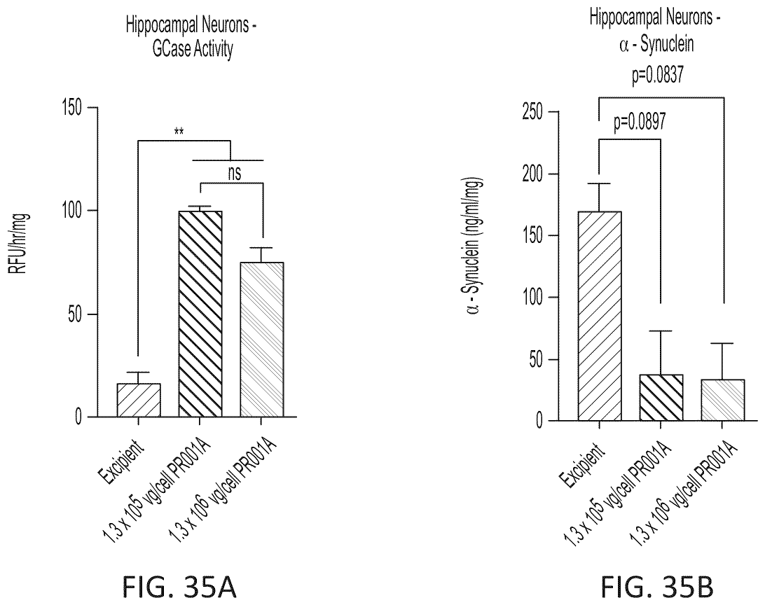

Mouse hippocampal neurons transduced with 1.3×10⁵ vg/cell or 1.3×10⁶ vg/cell PR001A showed increased GCase activity levels and trended to decreased total α-Synuclein levels (FIG. 35A and FIG. 35B).

In summary, PR001A transduction in cell lines and primary neuron cultures resulted in increased GCase activity. In HeLa cells and mouse hippocampal neurons, PR001A transduction also resulted in decreased α-Synuclein levels, supporting the link between GCase activity and α-Synuclein levels (Mazzulli et al., Cell. 2011;146(1):37-52).

### Example 3: In Vivo Assays Using Mutant Mice

This example describes in vivo assays of AAV vectors using mutant mice. In vivo studies of AAV vectors as above in mutant mice are performed using assays described, for example, by Liou et al. (2006) J. Biol. Chem. 281(7):4242-4253, Sun et al. (2005) J. Lipid Res. 46:2102-2113, and Farfel-Becker et al. (2011) Dis. Model Mech. 4(6):746-752.

The intrathecal or intraventricular delivery of vehicle control and AAV vectors (e.g., at a dose of 2×10¹¹ vg/mouse) are performed using concentrated AAV stocks, for example at an injection volume between 5-10 μL. Intraparenchymal delivery by convection enhanced delivery is performed.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 4: Chemical Models of Disease

This example describes in vivo assays of AAV vectors using a chemically-induced mouse model of Gaucher disease (e.g., the CBE mouse model). In vivo studies of these AAV vectors are performed in a chemically-induced mouse model of Gaucher disease, for example as described by Vardi et al. (2016) J Pathol. 239(4):496-509.

Intrathecal or intraventricular delivery of vehicle control and AAV vectors (e.g., at a dose of 2×10¹¹ vg/mouse) are performed using concentrated AAV stocks, for example with injection volume between 5-10 μL. Intraparenchymal delivery by convection enhanced delivery is performed. Peripheral delivery is achieved by tail vein injection.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 5: Clinical Trials in PD, LBD, Gaucher Disease Patients

In some embodiments, patients having certain forms of Gaucher disease (e.g., GD1) have an increased risk of developing Parkinson's disease (PD) or Lewy body dementia (LBD). This Example describes clinical trials to assess the safety and efficacy of rAAVs as described by the disclosure, in patients having Gaucher disease, PD and/or LBD.

Clinical trials of such vectors for treatment of Gaucher disease, PD and/or LBD are performed using a study design similar to that described in Grabowski et al. (1995) Ann. Intern. Med. 122(1):33-39.

### Example 6: Treatment of Peripheral Disease

In some embodiments, patients having certain forms of Gaucher disease exhibit symptoms of peripheral neuropathy, for example as described in Biegstraaten et al. (2010) Brain 133(10):2909-2919.

This example describes in vivo assays of AAV vectors as described herein for treatment of peripheral neuropathy associated with Gaucher disease (e.g., Type 1 Gaucher disease). Briefly, Type 1 Gaucher disease patients identified as having signs or symptoms of peripheral neuropathy are administered a rAAV as described by the disclosure. In some embodiments, the peripheral neuropathic signs and symptoms of the subject are monitored, for example using methods described in Biegstraaten et al., after administration of the rAAV.

Levels of transduced gene products as described by the disclosure present in patients (e.g., in serum of a patient, in peripheral tissue (e.g., liver tissue, spleen tissue, etc.)) of a patient are assayed, for example by Western blot analysis, enzymatic functional assays, or imaging studies.

### Example 7: Treatment of CNS Forms

This example describes in vivo assays of rAAVs as described herein for treatment of CNS forms of Gaucher disease. Briefly, Gaucher disease patients identified as having a CNS form of Gaucher disease (e.g., Type 2 or Type 3 Gaucher disease) are administered a rAAV as described by the disclosure. Levels of transduced gene products as described by the disclosure present in the CNS of patients (e.g., in serum of the CNS of a patient, in cerebrospinal fluid (CSF) of a patient, or in CNS tissue of a patient) are assayed, for example by Western blot analysis, enzymatic functional assays, or imaging studies.

### Example 8: Gene Therapy of Parkinson's Disease in Subjects Having Mutations in GBA1

This example describes administration of a recombinant adeno-associated virus (rAAV) encoding GBA1 to a subject having Parkinson's disease characterized by a mutation in GBAI gene.

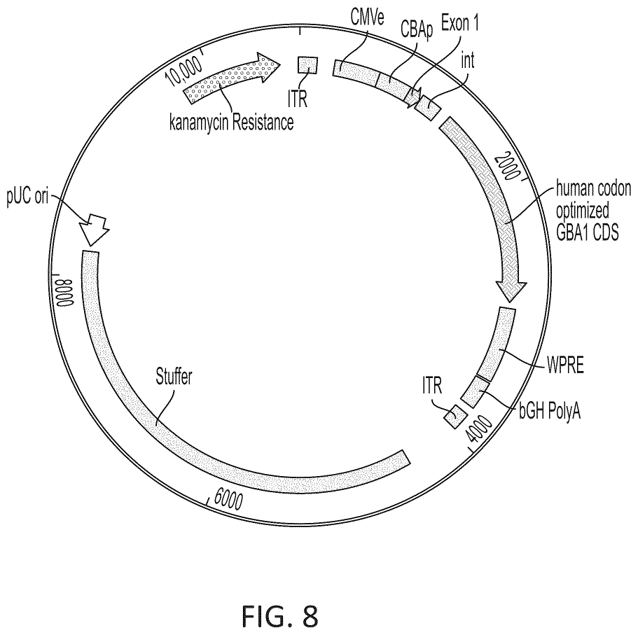

The rAAV vector insert contains the CBA promoter element (CBA), consisting of four parts: the CMV enhancer (CMVe), CBA promoter (CBAp), Exon 1, and intron (int) to constitutively express the codon optimized coding sequence (CDS) of human GBA1 (maroon). The 3′ region also contains a Woodchuck hepatitis virus Posttranscriptional Regulatory Element (WPRE) followed by a bovine Growth Hormone polyA signal (bGH polyA) tail. The flanking ITRs allow for the correct packaging of the intervening sequences. Two variants of the 5′ ITR sequence (FIG. 7, inset box, bottom sequence) were evaluated; these variants have several nucleotide differences within the 20-nucleotide “D” region of the ITR, which is believed to impact the efficiency of packaging and expression. The rAAV product contains the “D” domain nucleotide sequence shown in FIG. 7 (inset box, top sequence). A variant vector, harbors a mutant “D” domain (termed an “S” domain herein, with the nucleotide changes shown by shading), performed similarly in preclinical studies. The backbone contains the gene to confer resistance to kanamycin as well as a stuffer sequence to prevent reverse packaging. A schematic depicting the rAAV vector is shown in FIG. 8. The rAAV vector is packaged into an rAAV using AAV9 serotype capsid proteins.

GBA1-rAAV is administered to a subject as a single dose via a fluoroscopy guided sub-occipital injection into the cisterna magna (intracisternal magna; ICM). One embodiment of a dosing regimen study is as follows:

### Example 8.1: In Vivo Pharmacology Studies with rAAV Encoding Gcase

Initial studies were conducted in a chemical mouse model involving daily delivery of conduritol-P-epoxide (CBE), an inhibitor of GCase to assess the efficacy and safety of the PR001A rAAV vector (AAV9.CBA.GBA1.A) (schematic of a plasmid encoding the vector provided in FIG. 55), comprising the codon-optimized coding sequence of human GBA1 (SEQ ID NO:15), and a PR001B rAAV S-variant construct (as described further below). Additionally, initial studies were performed in a genetic mouse model, which carries a homozygous GBA1 mutation and is partially deficient in saposins (4L/PS-NA). Additional dose-ranging studies in mice and nonhuman primates (NHPs) are conducted to further evaluate vector safety and efficacy.

These mouse models exhibit phenotypes characteristic of nGD (neuronopathic Gaucher disease) and PD-GBA (having Parkinson's disease characterized by a mutation in GBA1 gene), including reduced GCase activity, accumulation of the glycolipid substrates of GCase, deficits in motor behavior, and neuropathological changes including astrogliosis and microgliosis, reflecting inflammation. Intracerebroventricular injection of PR001A suppressed all of these disease-associated phenotypes. Additionally, the 4L/PS-NA mouse model displayed accumulation of α-Synuclein, and ICV administration of PR001A in the 4L/PS-NA model reduced the accumulation of α-Synuclein.

Two slightly different versions of the 5′ inverted terminal repeat (ITR) in the AAV backbone were tested to assess manufacturability and transgene expression (FIG. 7). The 20 bp “D” domain within the 145 bp 5′ ITR is thought to be necessary for optimal viral vector production, but mutations within the “D” domain have also been reported to increase transgene expression in some cases. Thus, in addition to the PR001A viral vector, which harbors an intact “D” domain, a second vector form (PR001B) with a mutant D domain (termed an “S” domain herein) was also evaluated. Both PR001A rAAV and variant PR001B rAAV express the same transgene. While both vectors produced virus that was efficacious in vivo as detailed below, the PR001A rAAV, which contains a wild-type “D” domain, was selected for further development.

The nonclinical in vivo pharmacology (efficacy) studies are summarized in Table 14. A total of 10 studies were completed; the 4 principal studies are discussed in detail in subsequent sections.

### Example 8.1.1: CBE Mouse Model Studies

Overview of the CBE model

In the CBE chemical mouse model, a pharmacological inhibition of GCase activity is achieved using a selective and irreversible covalent competitive inhibitor of GCase, leading to glycolipid (GluCer and GluSph) accumulation, neuropathological changes including astrogliosis and microgliosis, and motor behavior deficits (Manning-Bo\check{g} et al., Neurotoxicology. 2009;30(6):1127-32; Farfel-Becker et al., Dis Model Mech. 2011;4(6):746-52; Rocha et al., Antioxid Redox Signal. 2015;23(6):550-64).

CBE is a pharmacological inhibitor of GCase, and mice treated with CBE display phenotypes consistent with GCase loss-of-function. By varying CBE dosage and, thus, the degree of GCase inhibition in vivo, it is possible to recapitulate the varied degrees of enzyme deficiency seen in different GBA1-associated disorders, thereby modulating the severity of the resulting phenotype. For this reason, the CBE mouse model has significant technical advantages over genetic models of GCase deficiency, making it an attractive model for PD-GBA. The systemic reduction in GCase activity in the CBE model recapitulates the human disease as patients with PD-GBA present with a reduction in GCase activity throughout the CNS and peripheral organs. It is expected that this model will underestimate the effects of PR001A since CBE will inhibit both endogenous GCase activity as well as exogenous GCase activity resulting from PR001A treatment.

### Study PRV-2017-001: CBE Dose-Ranging Study

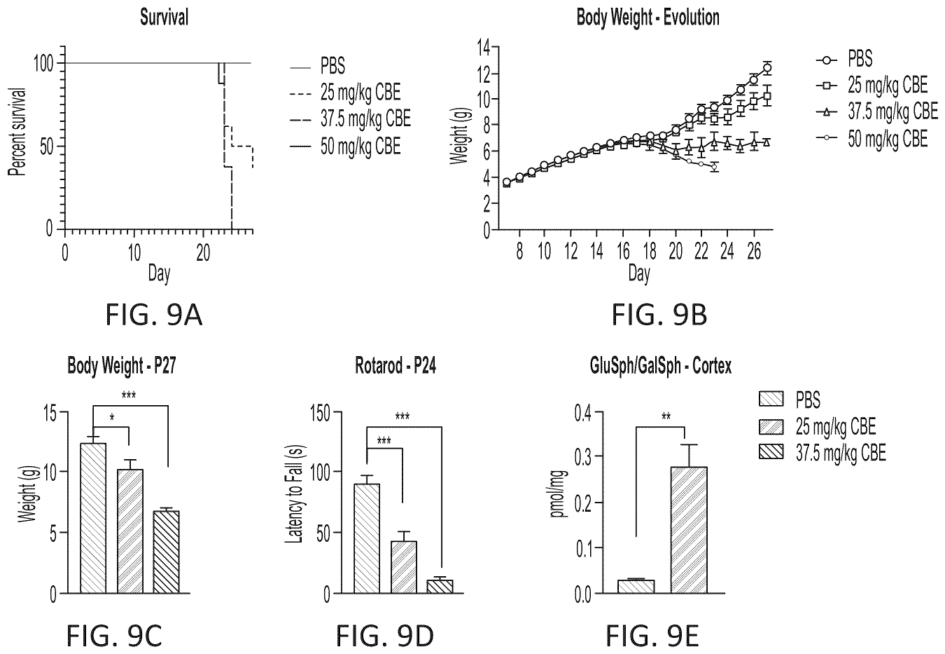

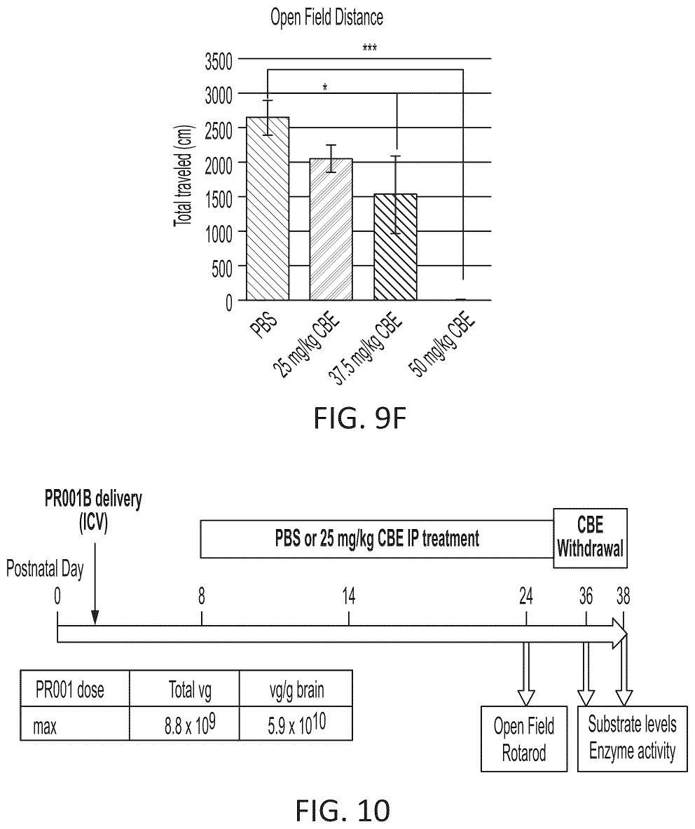

To establish the CBE model of GCase deficiency, juvenile mice were dosed with CBE, a specific inhibitor of GCase. Mice were given CBE by IP injection daily, starting at postnatal day 8 (P8). Three different CBE doses (25 mg/kg, 37.5 mg/kg, 50 mg/kg) or daily intraperitoneal (IP) vehicle (PBS) were tested to establish a model that exhibits a behavioral phenotype (FIG. 9A-FIG. 9F). Higher doses of CBE led to lethality in a dose-dependent manner. All mice treated with 50 mg/kg CBE died by P23, and 5 of the 8 mice treated with 37.5 mg/kg CBE died by P27 (FIG. 9A). There was no lethality in mice treated with 25 mg/kg CBE. Mice treated with CBE showed a failure to gain weight that correlated with CBE dose. At P27, the end of the in-life portion of the study, the weight difference was statistically significant between control animals and those treated with either 25 or 37.5 mg/kg CBE; no mice treated with 50 mg/kg CBE survived to P27 (FIG. 9B, FIG. 9C). Whereas CBE-injected mice showed no general motor deficits in the open field assay (traveling the same distance and at the same velocity as mice given PBS; FIG. 9F), CBE-treated mice exhibited a motor coordination and balance deficit as measured by the rotarod assay (FIG. 9D).

Mice surviving to the end of the study were sacrificed on the day after their last CBE dose (P27, “Day 1”) or after three days of CBE withdrawal (P29, “Day 3”). Lipid analysis was performed on the cortex of mice given 25 mg/kg CBE to evaluate the accumulation of GCase substrates in both the Day 1 and Day 3 cohorts. GluSph and GalSph levels (measured in aggregate in this example) were significantly accumulated in the CBE-treated mice compared to PBS-treated controls, consistent with GCase insufficiency (FIG. 9E).

In summary, a dose of 25 mg/kg CBE injected IP daily resulted in motor behavior deficits and accumulation of GCase substrates (aggregate of GluSph and GalSph levels), which is consistent with inhibition of GCase activity. Therefore, the 25 mg/kg dose was selected for subsequent studies since this recapitulated the core features of the human disease while permitting longer studies to evaluate persistence of vector.

### Study PRV-2018-002: Efficacy of PR001B in the CBE Model

Based on the study described above, the 25 mg/kg CBE dose was selected since it produced behavioral deficits without impacting survival. For all nonclinical mouse studies, intracerebroventricular (ICV) injection was chosen as the route of administration (ROA). As intra-cisterna magna (ICM) injection (the intended clinical ROA) is technically difficult in mice, ICV injection was deemed the most suitable alternative approach to recapitulate the ICM delivery of the therapeutic agent into the cerebrospinal fluid (CSF). To achieve widespread GBA1 distribution throughout the brain and transgene expression during CBE treatment, 4μL vehicle (dPBS+0.001% Pluronic F68, “dPBS”) or 8.8×10⁹ vg (5.9×10¹⁰ vg/g brain, based on a brain weight of 150 mg) PR001B was delivered via ICV injection at P3 and daily IP injection of PBS or 25 mg/kg CBE treatment was initiated at P8 (FIG. 10). To determine if CBE treatment would completely mask the effect of PR001B, half the animals were sacrificed on P36, 1 day after their final CBE injection, while the other half underwent CBE withdrawal and were sacrificed on P38, 3 days after their final CBE injection. For all measures, the groups were combined for analysis correcting for day of collection as a covariate.

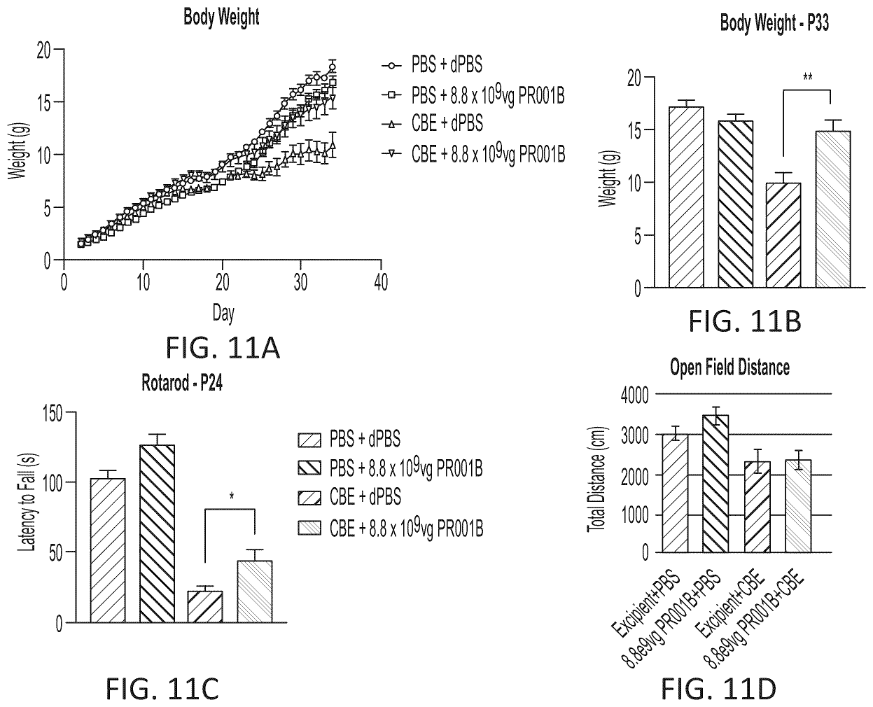

The CBE-treated mice showed decreased body weight evolution that was attenuated with PR001B treatment (FIG. 11A; FIG. 11B). CBE-treated mice that received rAAV performed statistically significantly better on the rotarod than those that received excipient (FIG. 11C). Mice in the variant vector treatment group did not differ from excipient treated mice in terms of total distance traveled during testing (FIG. 11D).

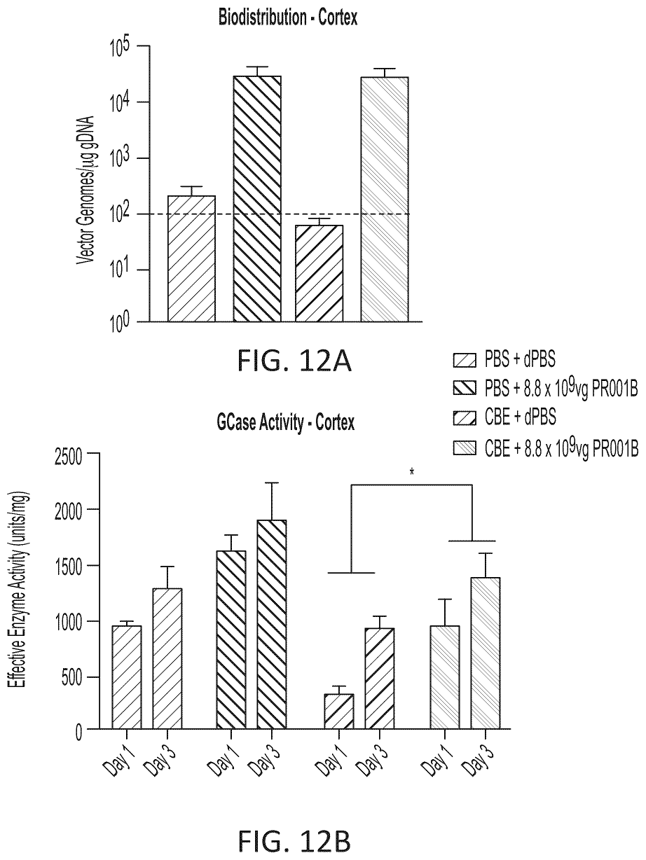

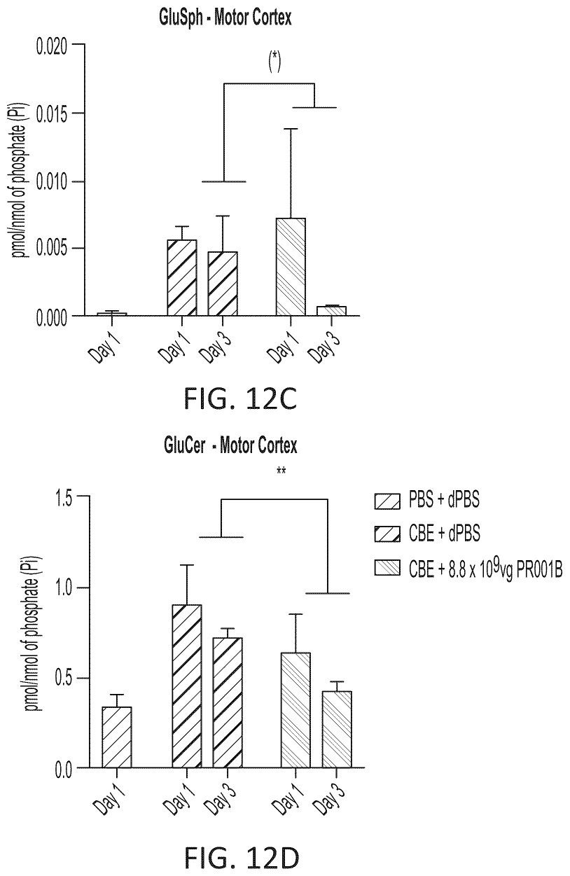

At the completion of the in-life study, half of the mice were sacrificed the day after the last CBE dose (P36, “Day 1”) or after three days of CBE withdrawal (P38, “Day 3”) for biochemical analysis (FIG. 12B-FIG. 12D). Using a fluorometric enzyme assay performed in biological triplicate, GCase activity was assessed in the cortex. GCase activity was increased in mice that were treated with PR001B rAAV, while CBE treatment reduced GCase activity (FIG. 12B). Additionally, mice that received both CBE and PR001B rAAV had GCase activity levels that were similar to the PBS-treated group, indicating that delivery of rAAV is able to overcome the inhibition of GCase activity induced by CBE treatment. Lipid analysis was performed on the motor cortex of the mice to examine levels of the substrates GluCer and GluSph. Both lipids accumulated in the brains of mice given CBE, and rAAV treatment significantly reduced GluCer accumulation and tended to reduce GluSph accumulation (FIG. 12C; FIG. 12D).

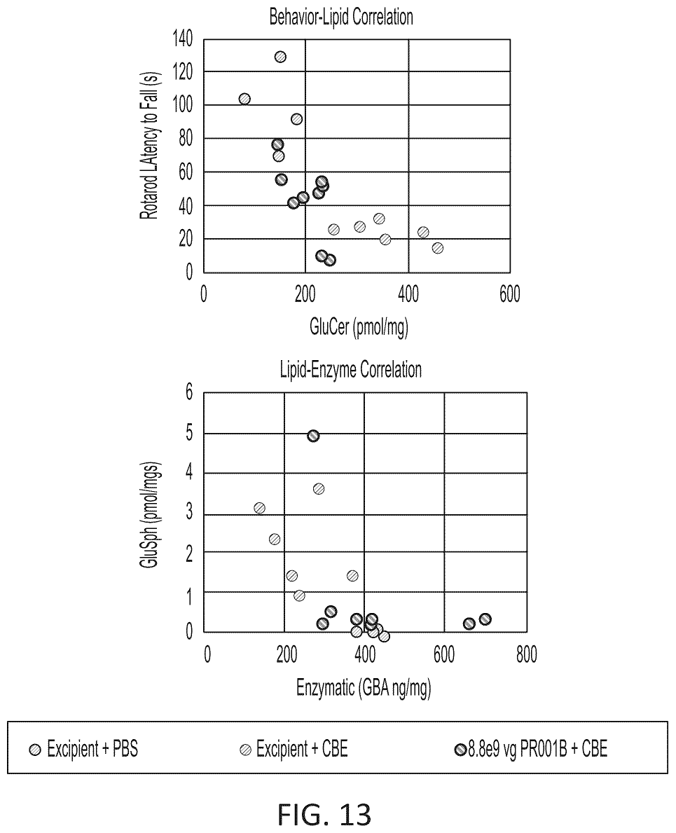

Lipid levels were negatively correlated with both GCase activity and performance on the Rotarod across treatment groups. The increased GCase activity after rAAV administration was associated with substrate reduction and enhanced motor function (FIG. 13).

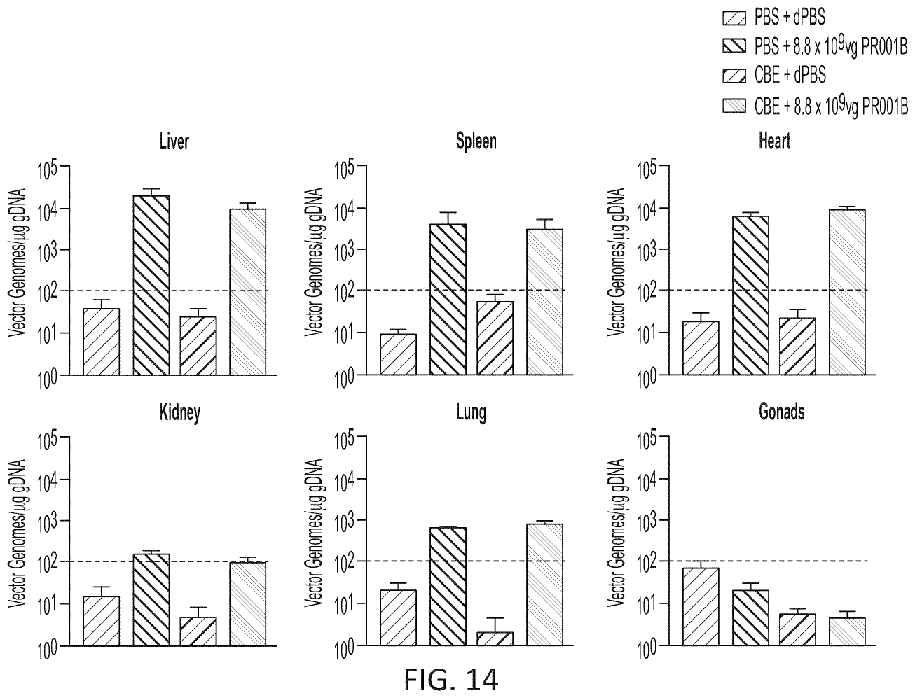

As shown in FIG. 14, preliminary biodistribution was assessed by vector genome presence, as measured by qPCR (with >100 vector genomes per 1 μg genomic DNA defined as positive). Mice that received PR001B rAAV, both with and without CBE, were positive for rAAV vector genomes in the cortex (FIG. 12A), indicating that ICV delivery results in rAAV delivery to the cortex. Additionally, vector genomes were detected in the liver, spleen, heart, and lung, with lower levels in the kidney, and none in the gonads (FIG. 14). For all measures, there was no statistically significant difference between the Day 1 and Day 3 groups (data not shown).

In summary, at a dose of 8.8×10⁹ vg (5.9×10¹⁰ vg/g brain) injected ICV, PR001B was distributed in the brain and peripheral tissues, and enzymatically active GCase was expressed in the brain. PR001B improved the biochemical (i.e., glycolipid levels) deficits and performance on rotarod. Because CBE withdrawal was not necessary in order to see the effects of PR001B, mice were sacrificed 1 day following the last CBE dose in all future studies.

### Study PRV-2018-005: Dose-Ranging PR001A in CBE Model

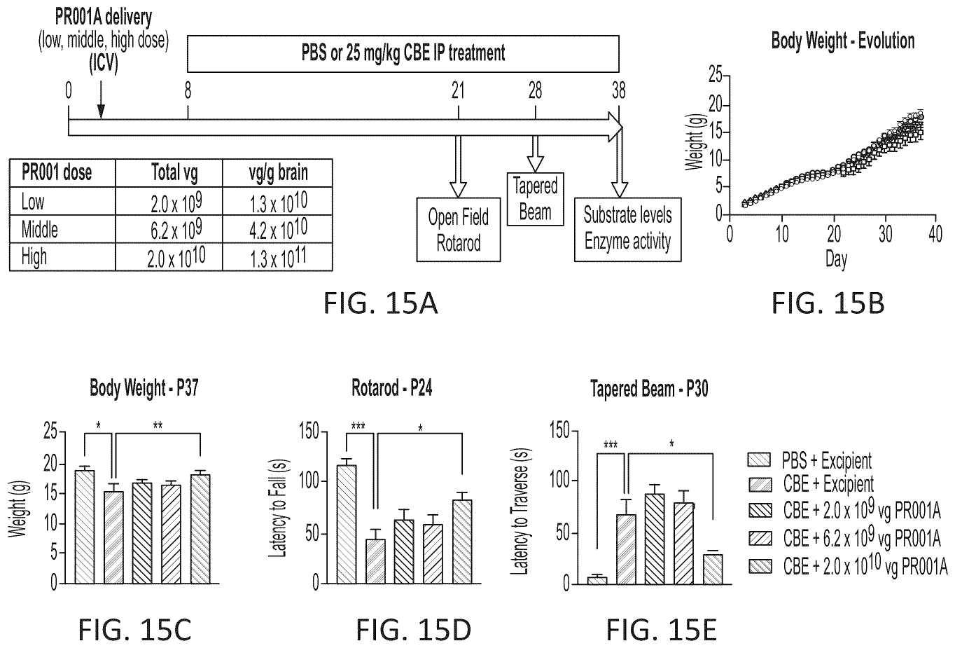

A schematic showing an illustrative dose-ranging study design is provided in FIG. 15A.

A larger study in the CBE model further explored efficacious doses of PR001 rAAV in the CBE model. Using the 25 mg/kg CBE dose model, excipient or PR001 rAAV was delivered via ICV at P3, and daily IP PBS or CBE treatment initiated at P8. Given the similarity between the groups with and without CBE withdrawal observed in the previous studies, all mice were sacrificed one day after the final CBE dose (P38-40). The effect of three different rAAV doses was assessed, resulting in the following five groups, with 10 mice (5M/5F) per group:
Excipient ICV+PBS IP
Excipient ICV+25 mg/kg CBE IP
2.0e9 vg (1.3e10 vg/g brain) rAAV ICV+25 mg/kg CBE IP
6.2e10 vg (4.2e10 vg/g brain) rAAV ICV+25 mg/kg CBE IP
2.0e10 vg (1.3e11 vg/g brain) rAAV ICV+25 mg/kg CBE IP.

The CBE-treated animals gained weight at a lower rate than control animals, a typical observation in this animal model. At the highest dose, PR001A corrected the CBE treatment-related failure to gain weight. Additionally, this dose resulted in a statistically significant improvement on the rotarod and tapered beam tasks, compared to the CBE-treated group that did not receive PR001A (FIG. 15B-FIG. 15E). Brain GCase activity was positively correlated with performance on the rotarod. Lethality was observed in several groups, including both excipient-treated and rAAV-treated group.

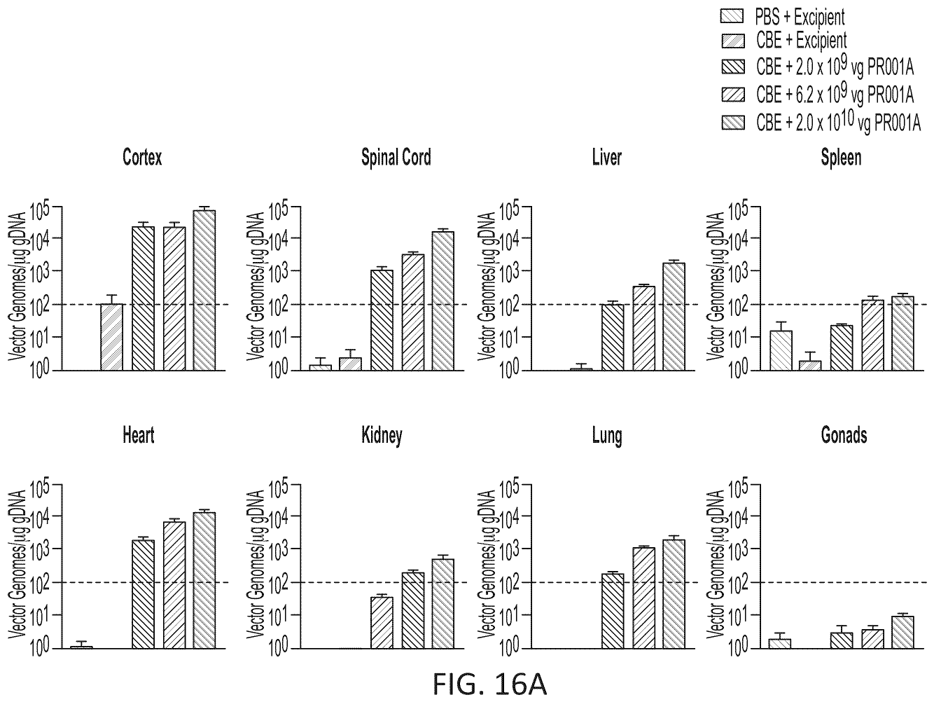

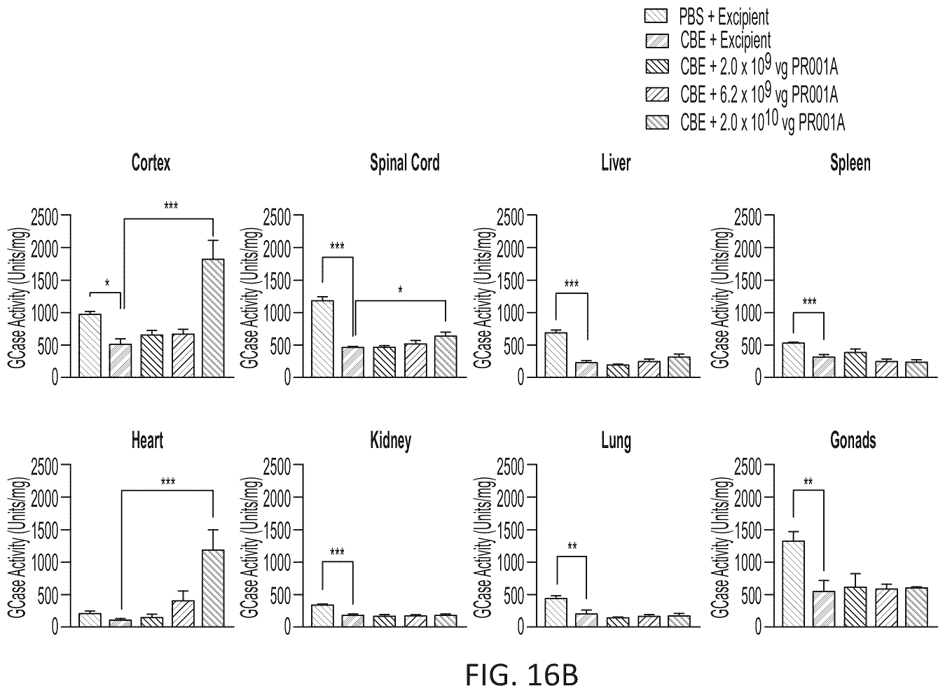

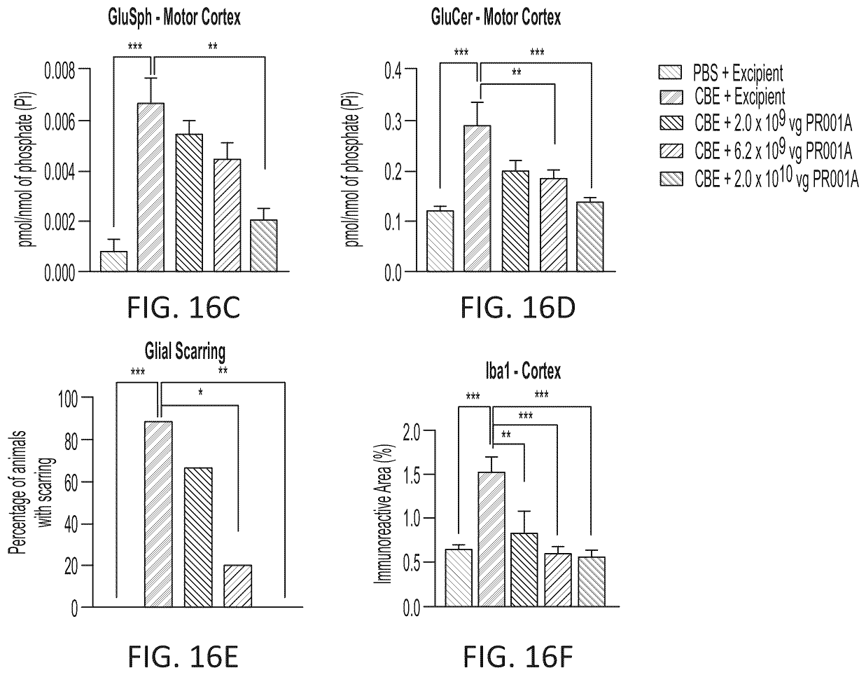

At the completion of the in-life study, mice were sacrificed for biodistribution and biochemical analysis (FIG. 16A-FIG. 16D). Of the tissues examined, brain, spinal cord, liver, spleen, heart, kidney, and lungs were positive for vector genomes at the middle and highest doses. The brain, spinal cord, lung, and heart were also positive at the low dose (FIG. 16A). Gonads were not positive at any dose. Effective GCase activity, evaluated by measuring enzymatic activity in all tissues using a fluorometric assay, was reduced by up to 60% following treatment with CBE (FIG. 16B). At the highest dose of PR001A, GCase activity was significantly increased in the brain, spinal cord, and heart. Note that because CBE treatment inhibits the activity of PR001A-encoded GCase to the same extent as endogenous GCase by approximately 50%, CBE model studies likely underestimate the potency of PR001A as measured by GCase activity by approximately 2-fold. The CBE-treated mice exhibited accumulation of GluCer and GluSph in the brain cortex. The high dose of PR001A reduced their accumulation (FIG. 16C; FIG. 16D).

Reactive astrogliosis and microglial activation are prominent inflammatory aspects of the CNS pathology described in neuronopathic GD and PD-GBA patients (Wong et al 2004; Ginns et al 2014). In this study, CBE-treated mice displayed glial scarring, a manifestation of reactive astrogliosis, in the cerebral cortex, consistent with prior studies showing CNS activation in the context of CBE (Sun et al 2011). PR001A treatment led to a statistically significant, dose-dependent reduction of the glial scarring phenotype (FIG. 16E). Thus, PR001 treatment suppresses the neuropathology associated with GCase deficiency in the CNS. A full description of the histopathological findings from this study are discussed in the toxicology section.

Immunohistochemistry was performed for GCase and ionizing calcium-binding adaptor molecule 1 (Iba1; a marker of microgliosis) expression in the cortex (FIG. 16F) (Wong et al 2004; Vitner et al 2016). The expression of GCase was significantly increased in all mice treated with PR001A compared to CBE+excipient-treated animals and correlated with the dose delivered. Iba1 staining was significantly reduced in a dose-dependent manner in mice treated with PR001A compared with mice receiving CBE+excipient, in which Iba1 staining was significantly increased compared with mice receiving PBS.

In summary, the results of Study PRV-2018-005 show that ICV administration of PR001A at 3 dose levels led to broad vector genome biodistribution, increase in GCase activity, improvement on motor behavioral endpoints, and reduction in glycolipid accumulation. Two different measures of neuroinflammation (microgliosis and astrogliosis) showed a dose dependent, statistically significant decrease in mice treated with PR001A. The CBE model inherently underestimates the potency of PR001A since CBE also inhibits enzyme activity due to PR001A treatment. Taken together, these results indicate that ICV administration of PR001A at 2.0×10¹⁰ vg (1.3×10¹¹ vg/g brain) was effective in the CBE mouse model. A trend towards efficacy was observed at lower doses of PR001A in a subset of endpoints.

### Study PRV-2018-007: Long-Term PR001A Effects in CBE Model

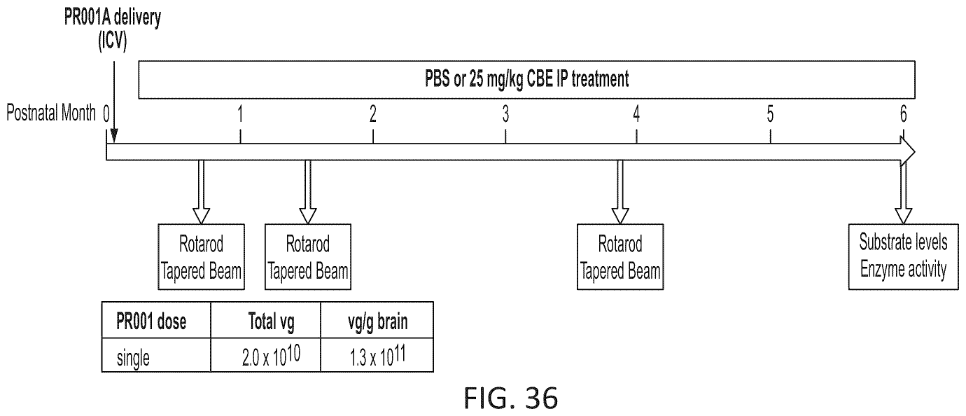

This study assessed the persistence of PR001A vector copy number biodistribution and the durability of PR001A-mediated expression of GCase in the CBE mouse model. A single dose of excipient or PR001A was delivered via ICV at P3, and daily IP PBS or CBE treatment was initiated at P8 and continued until P183 through P185 (FIG. 36). All mice were sacrificed 1 day after the final CBE dose. No lethality was observed in any group.

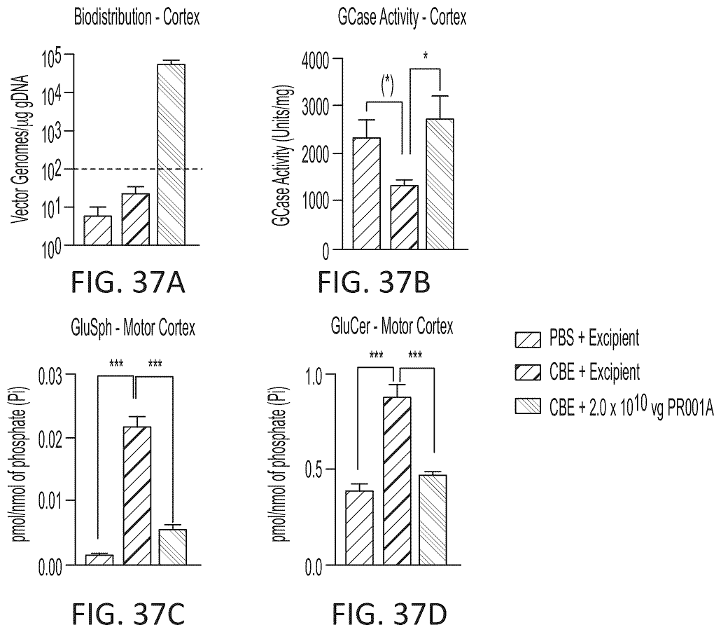

A single ICV dose of PR001A in CBE-treated mice led to the presence of vector genome copies 6 months after dosing (FIG. 37A) at levels comparable to levels seen at approximately 1 month after ICV dosing (see Study PRV-2018-005). Chronic CBE treatment resulted in reduced GCase activity levels; GCase activity was nearly normalized in the mice receiving PR001A, indicating that a single dose of PR001A leads to a durable expression of GCase (FIG. 37B). Six month CBE-treated mice showed a pronounced accumulation of the glycolipid substrates GluCer and GluSph in the cerebral cortex, compared to those receiving 1 month of CBE treatment. A single administration of PR001A at P3 resulted in a significant reduction of GluCer and GluSph levels to near wildtype levels (FIG. 37C; FIG. 37D).

### Study PRV-2018-008: Additional Dose-Ranging PR001A in CBE Model

This study was intended to evaluate additional doses of PR001A to determine the minimum effective dose and examine higher doses for tolerability. However, due to an unexpected dosing deviation, this study replicated the doses from PRV-2018-005. Following a similar design as PRV-2018-005 (FIG. 15A), 4 μL excipient or PR001A was delivered via ICV at P3, and daily IP PBS or CBE treatment was initiated at P8 and continued until P51 through P53.

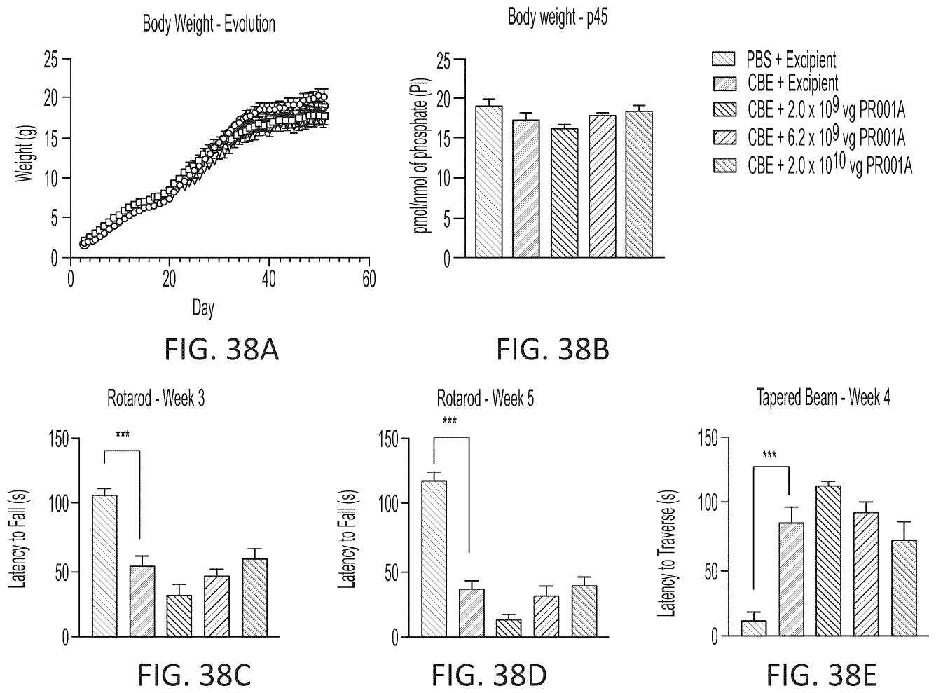

Unlike previous studies, CBE treatment did not lead to a significant change in body weight. Although CBE treatment resulted in significantly poorer performance on the rotarod and tapered beam, treatment with PR001A did not significantly alter this performance (FIG. 38A-FIG. 38E).

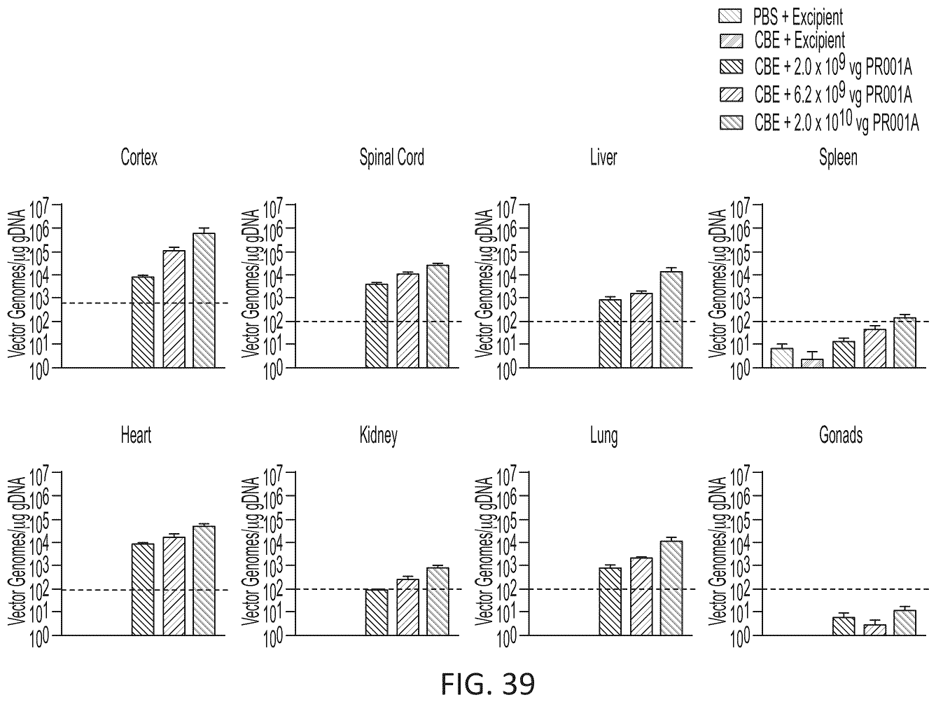

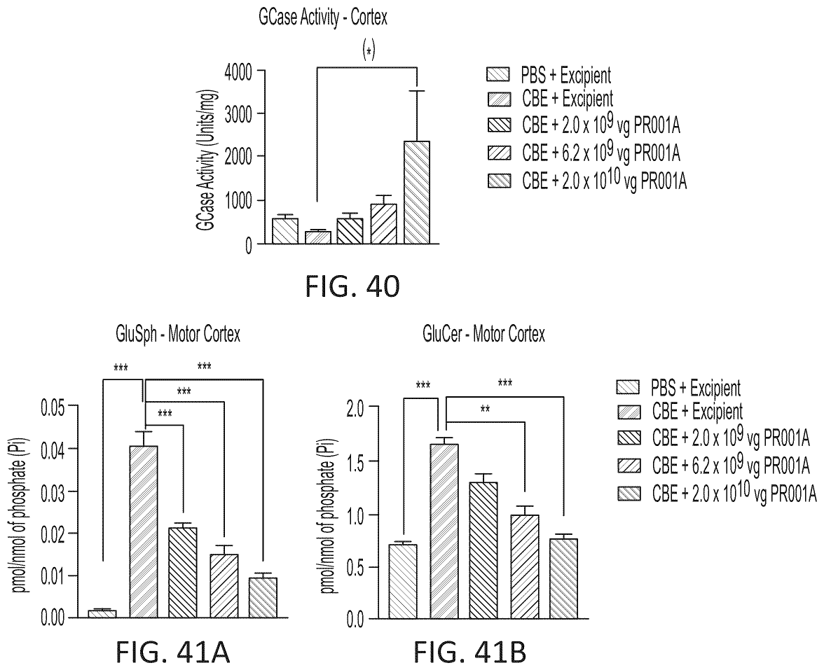

Of the tissues examined, brain, spinal cord, liver, heart, and lungs were positive for PR001A at all dose levels. The kidney was also positive at the middle and highest doses, while the spleen was only positive at the highest dose. Gonads were also examined but were not positive at any dose level (FIG. 39). GCase activity was assessed only in select organs. In the cerebral cortex, the low and middle dose of PR001A restored GCase activity levels to the equivalent of PBS+excipient levels or greater, although this did not reach statistical significance. The high dose of PR001A trended toward a significant increase in GCase activity compared to levels in CBE+excipient-treated animals (FIG. 40).

Consistent with the other studies in this model, CBE-treated mice exhibited accumulation of GluSph and GluCer in the brain, which was reduced by administering PR001A (FIG. 41; FIG. 41B). In a dose-dependent manner, all doses of PR001A significantly decreased GluSph levels while the middle and high dose significantly decreased GluCer levels.

This study confirmed the findings from PRV-2018-005, showing that PR001A treatment results in broad biodistribution and a robust elevation of GCase activity that significantly reduces the glycolipid substrate accumulation caused by CBE treatment. This study did not replicate the behavioral phenotypes observed in PRV-2018-005; however, these phenotypes are known to be variable and less reliable in mice.

### PRV-2018-025: Further Dose-Ranging PR001A in CBE Model

Given the study deviation in PRV-2018-008, an additional study was performed in the CBE model to expand on previous dose-ranging studies. The ICV dosing of PR001A and IP injection of PBS or CBE followed the same protocol as PRV-2018-005. However, this study included a lower PR001A dose to examine the minimum effective dose and a higher dose to examine tolerability.

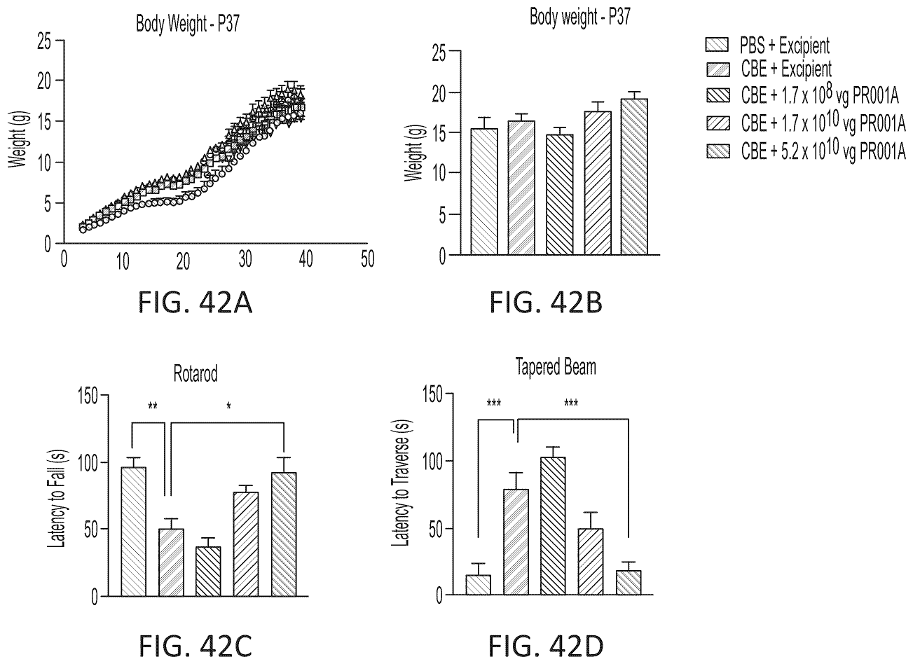

In this study, CBE treatment did not lead to a failure to gain weight over time; however, a statistically significant decrease in motor performance was observed in CBE+excipient animals in both the rotarod and tapered beam. Treatment with PR001A at 5.2×10¹⁰ vg significantly improved motor performance to nearly the same level as PBS+excipient animals. An improvement was also observed in animals treated with 1.7×10¹⁰ vg PR001A, though this did not reach significance (FIG. 42A-FIG. 42D).

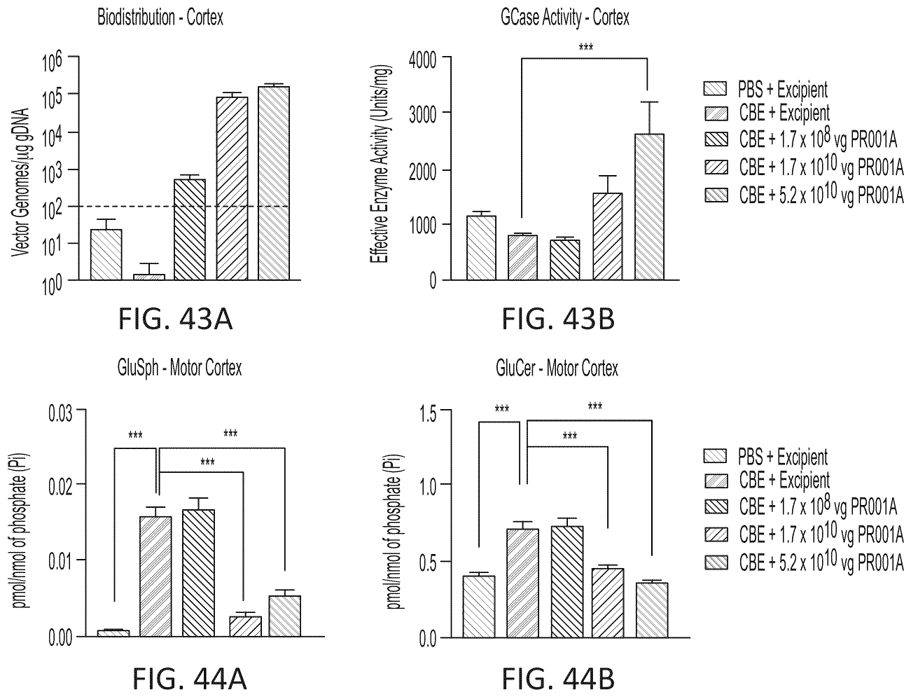

The cerebral cortex of animals treated with PR001A was positive for vector genomes at all doses, and treatment with 5.2×10¹⁰ vg PR001A led to a significant increase in GCase activity. Treatment with 1.7×10¹⁰ vg PR001A restored activity to near wildtype levels (FIG. 43A-FIG. 43B), although this did not reach statistical significance.

Consistent with the other studies in this model, CBE-treated mice exhibited an accumulation of GluSph and GluCer in the brain, which was significantly reduced by administering PR001A at either 1.7×10¹⁰ vg or 5.2×10¹⁰ vg (FIG. 44A-FIG. 44B).

This study confirmed and expanded on the findings from the previous studies in the CBE model. Although this study did not completely replicate the behavioral phenotypes observed in PRV-2018-005, nonsignificant improvements were seen in both rotarod and tapered beam with 1.7×10¹⁰ vg PR001A, and treatment with 5.2×10¹⁰ vg PR001A significantly improved performance in both tasks. Additionally, treatment at either dose decreased glycolipid substrate accumulation, confirming the results from the other CBE studies.

### Summary of CBE Model Studies

Results from CBE model studies show that PR001A can be effectively delivered to the CNS and also peripheral tissues by ICV injection. Within the CNS, ICV delivery of PR001A resulted in a consistent increase in GCase activity, a reduction of the glycolipid substrates GluCer and GluSph, a reduction of glial scarring, and improvement in some motor deficits. These effects, where assessed, persisted at 6 months post treatment.

### Example 8.1.2: 4L/PS-NA Genetic Mouse Model Studies

Overview of the 4L/PS-NA model

4L/PS-NA mice are an established genetic model of GD and PD-GBA (Sun et al., J Lipid Res. 2005;46(10):2102-13; Mazzulli et al., Cell. 2011;146(1):37-52; Xu et al., Mol Genet Metab. 2011;102(4):436-47). These mice are homozygous for the V394L mutation in GBA1 and additionally harbor mutations in PSAP, which encodes saposin C, an activator of GCase; the presence of a mutant GCase enzyme and the low levels of the GCase activator saposin C together lead to a severe reduction in GCase activity, accumulation of glycolipid substrates, as well as motor behavior deficits. These mice exhibit motor strength, coordination, and balance deficits, as evidenced by their performance in the beam walk, rotarod, and wire hang assays. Typically the lifespan of these mice is less than 22 weeks. The “control” mice in this study are homozygous for the V394L mutation in Gba1, but wild-type for the endogenous prosaposin gene, and thus harbor a more modest reduction in GCase activity. Note that because treatment with PR001A does not have an effect on saposin C, results obtained in the 4L/PS-NA mice likely underestimate the predicted effect in humans. Two studies were conducted with PR001A in these mice.

### Study PRV-2018-006: PR001A in 4L/PS-NA Genetic Model

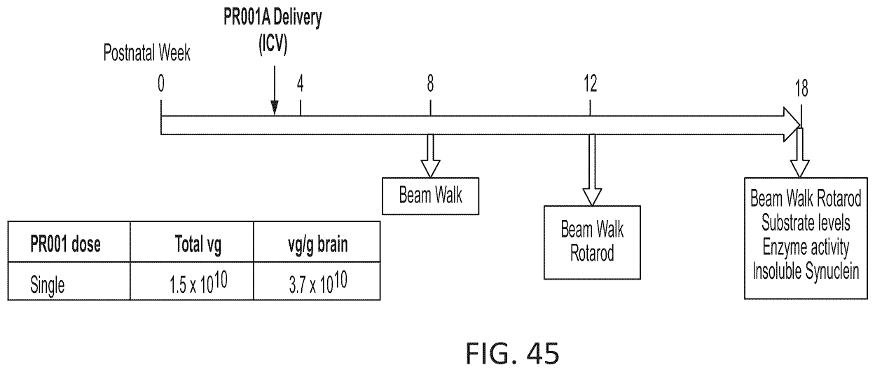

In Study PRV-2018-006, PR001A or excipient was delivered ICV to 3 to 4 week old 4L/PS-NA mice, and animals were sacrificed 15 weeks post-PR001A administration. A dose of 3 μL of undiluted vector (1.5×10¹⁰ vg total; 3.7×10¹⁰ vg/g brain) was administered (FIG. 45).

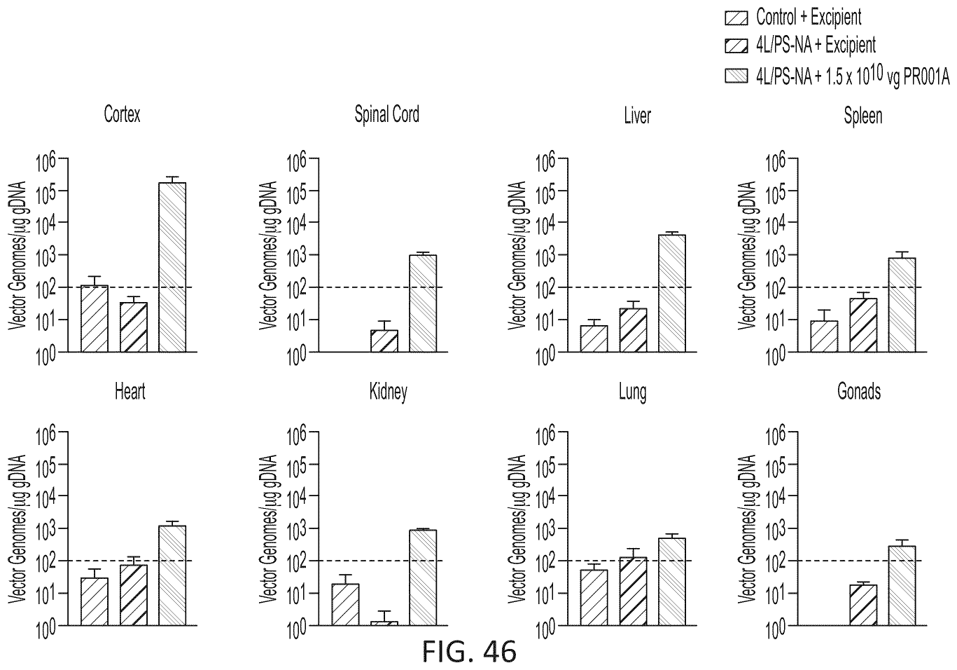

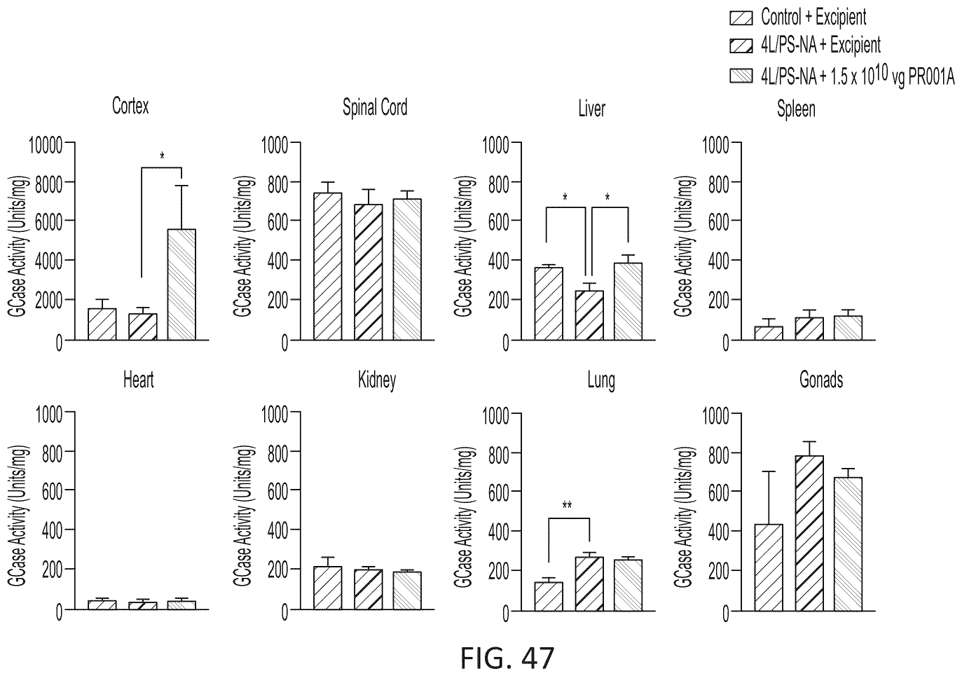

Progressive motor deficits were observed in the 4L/PS-NA mice, and treatment with PR001A resulted in a nonsignificant improvement on beam walk 5 and 9 weeks after treatment. At 15 weeks post treatment, there was no statistically significant difference among the groups. Biodistribution of PR001A vector genomes in the 4L/PS-NA mice was quantified approximately 15 weeks after dosing. All tissues examined, including cerebral cortex, spinal cord, liver, kidney, heart, lung, spleen, and gonads, were positive for vector genomes. (FIG. 46). Analysis of GCase activity in tissue lysates, evaluated using a fluorometric assay, revealed significant increases in effective GCase activity in the cortex and liver (FIG. 47).

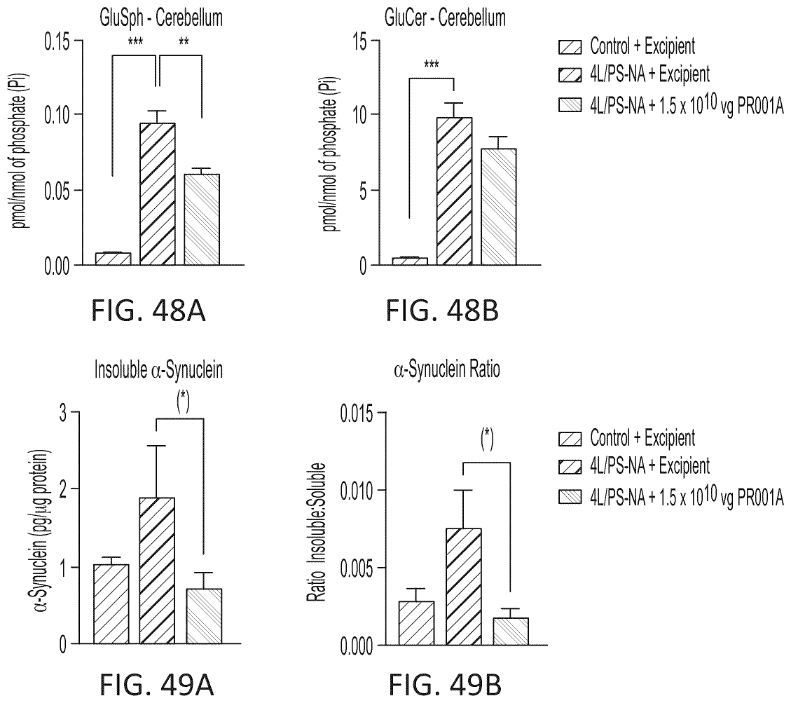

There was a statistically significant accumulation of GluSph and GluCer in the brain lysates from 4L/PS-NA mice relative to lysates from control animals. In the 4L/PS-NA mice, treatment with PR001A led to a statistically significant reduction in GluSph accumulation and a trend (P=0.16) towards a reduction in GluCer (FIG. 48A; FIG. 48B).

Prior studies have demonstrated increased accumulation of α-Synuclein protein in the cortex of the 4L/PS-NA mouse model, consistent with the proposed role of GCase in α-Synuclein pathology (Sun et al., J Lipid Res. 2005;46(10):2102-13; Mazzulli et al., Cell. 2011;146(1):37-52; Xu et al., Mol Genet Metab. 2011;102(4):436-47). Cerebral cortical levels of soluble and insoluble α-Synuclein were examined biochemically. In 4L/PS-NA mice treated with excipient, there was a nonsignificant increase in insoluble α-Synuclein and the ratio of insoluble to soluble α-Synuclein in the cerebral cortex; treatment with ICV PR001A reversed these effects (P=0.19, P=0.87, respectively) (FIG. 49A; FIG. 49B). These data are consistent with in vitro studies described in Example 2.1 that demonstrate reduced accumulation of α-Synuclein.

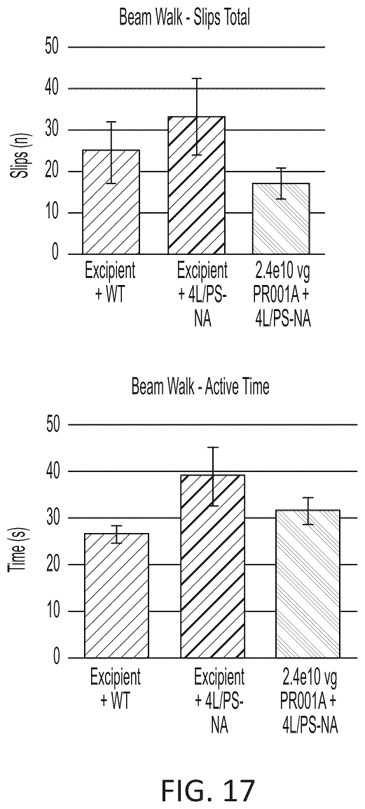

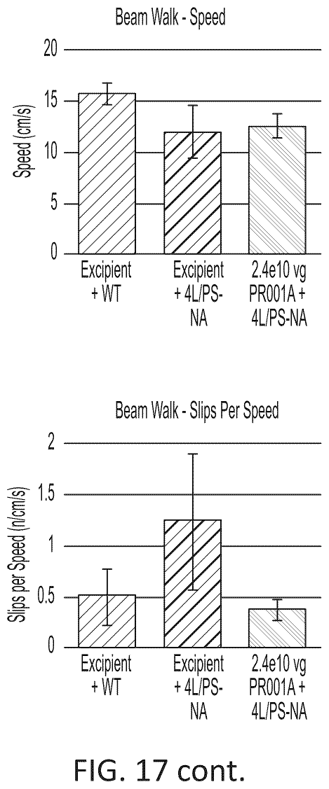

Motor performance by the beam walk test was assessed 4 weeks post-rAAV delivery. The group of mutant mice that received PR001A rAAV showed a trend towards fewer total slips and fewer slips per speed when compared to mutant mice treated with excipient, restoring motor function to near wild-type levels (FIG. 17).

### Study PRV-2018-011: Dose-Ranging PR001A in 4L/PS-NA Genetic Model

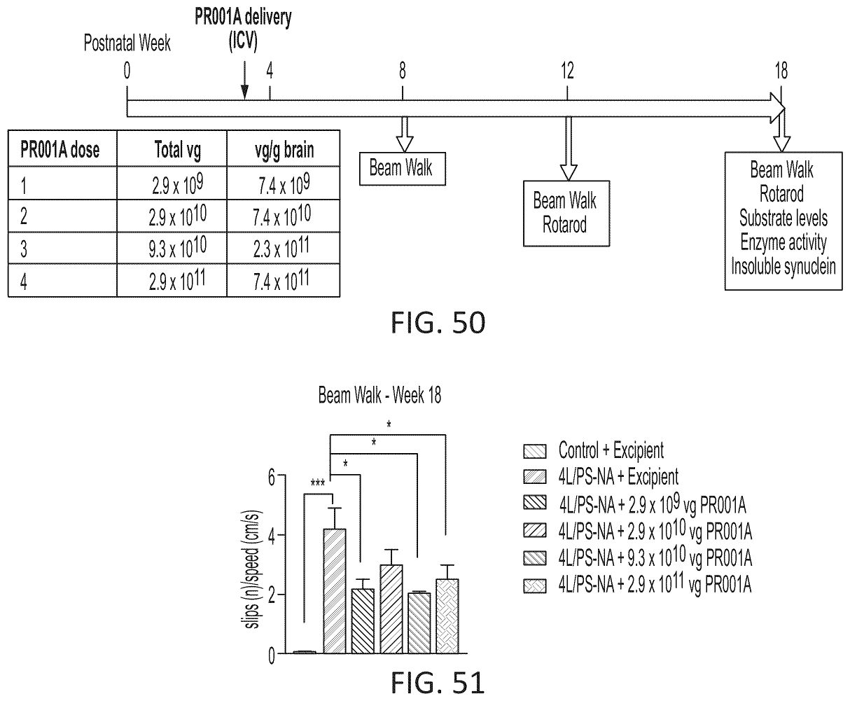

The second study with 4L/PS-NA mice explored a range of PR001A doses using a design similar to the one used in Study PRV-2018-006 (FIG. 50).

On the beam walk test, 4L/PS-NA mice performed significantly worse than control mice. 4L/PS-NA mice treated with 2.9×10¹¹ vg, 9.3×10¹⁰ vg, or 2.9×10¹⁰ vg PR001A showed significant improvement when compared to 4L/PS-NA mice treated with excipient at Week 18 (FIG. 51). There was no effect of PR001A at the earlier timepoints. There was no difference in rotarod test results between 4L/PS-NA mice and control mice, and PR001A treatment did not appear to have an effect on this outcome.

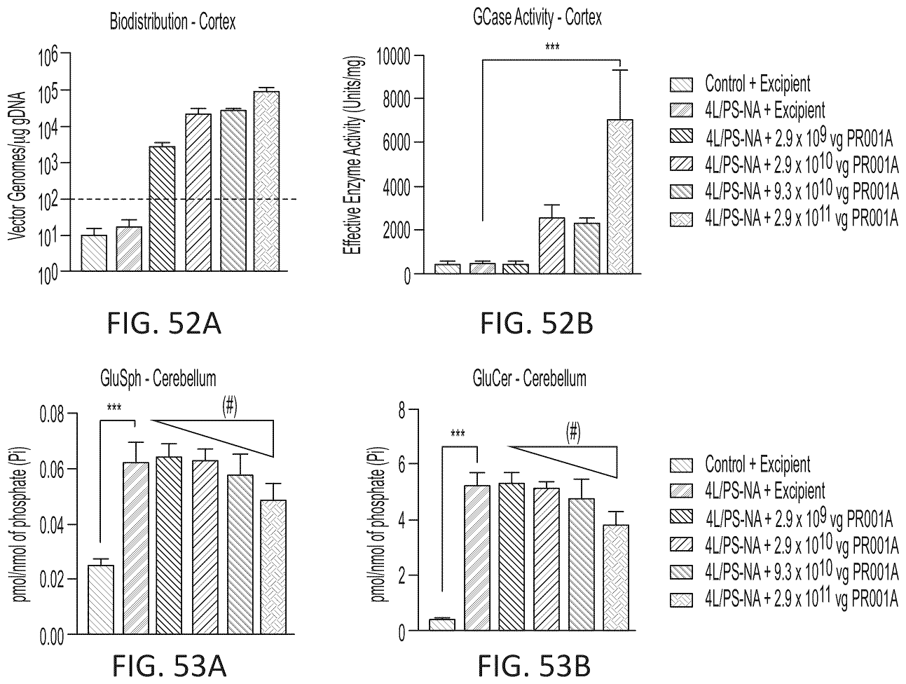

All PR001A treatment groups were positive for vector genomes in the cortex. Effective GCase activity, evaluated using a fluorometric assay, was measured in the cortex and was found to be significantly increased in mice treated with 2.9×10¹¹ vg PR001A (FIG. 52A; FIG. 52B).

Cerebral cortical and hippocampal levels of soluble and insoluble α-Synuclein were examined biochemically. There was no difference in these levels between 4L/PS-NA mice and control animals; published reports in the literature have shown variable α-Synuclein phenotypes.

There was a statistically significant accumulation of GluSph and GluCer in the cerebellum of 4L/PS-NA mice treated with excipient. Treatment with PR001A led to a dose-dependent trend to reduced levels of GluSph and a statistically significant dose-dependent reduction in GluCer (FIG. 53A; FIG. 53B).

### Summary of 4L/PS-NA Genetic Mouse Model

Although the 4L/PS-NA mice displayed variability with respect to the measured phenotypes across 2 studies, the overall data were consistent with the CBE model findings and published data: GCase deficiency was associated with an increased level of glycolipid substrates and motor behavioral deficits. Treatment with ICV PR001A strongly attenuated these phenotypes. In Study PRV-2018-006, insoluble α-Synuclein levels in the cerebral cortex were nonsignificantly increased in the 4L/PS-NA relative to control mice, as reported in published studies (Sun et al., J Lipid Res. 2005;46(10):2102-13; Mazzulli et al., Cell. 2011;146(1):37-52; Xu et al., Mol Genet Metab. 2011;102(4):436-47). Treatment with ICV PR001A reversed such accumulation, consistent with in vitro analyses disclosed herein. Taken together, these studies support the clinical development of PR001A.

### Example 8.1.3: In Vivo α-Synuclein Model Studies

Study PRV-2018-019 and PRV-2019-001: PR001A in α-Synuclein Transgenic Mice Treated with CBE

To further examine the effect of PR001A on α-Synuclein pathology, 2 studies were performed in dbl-PAC-Tg(SNCAA53T);Snca^{-/-} mice, which are homozygous for a human PD-associated α-Synuclein A53T mutant transgene on a Snca knockout background (Snca encodes the murine α-Synuclein protein). These mice are reported to display gastrointestinal phenotypes and subtle motor abnormalities between 6 to 12 months of age but not widespread α-Synuclein pathology in the brain (Kuo et al., Hum Mol Genet. 2010;19(9):1633-50). Previous studies in human α-Synuclein A53T transgenic mouse models have reported that the treatment of such mice with CBE leads to elevated α-Synuclein levels (Rockenstein et al., Hum Mol Genet. 2016; 25(13):2645-60; Papadopoulos et al., Hum Mol Genet. 2018;27(10):1696-1710). Due to these published findings, and to validate the effects of GCase deficiency in this model, we treated these mice with CBE. At 9 to 10 weeks of age, mice were treated with 10 μL of excipient or 2.9×10¹¹ vg (7.4×10¹¹ vg/g brain, based on a brain weight of 400 mg) PR001A via ICV injection. Two weeks post-ICV treatment, IP PBS or 100 mg/kg CBE was given daily for 1 week.

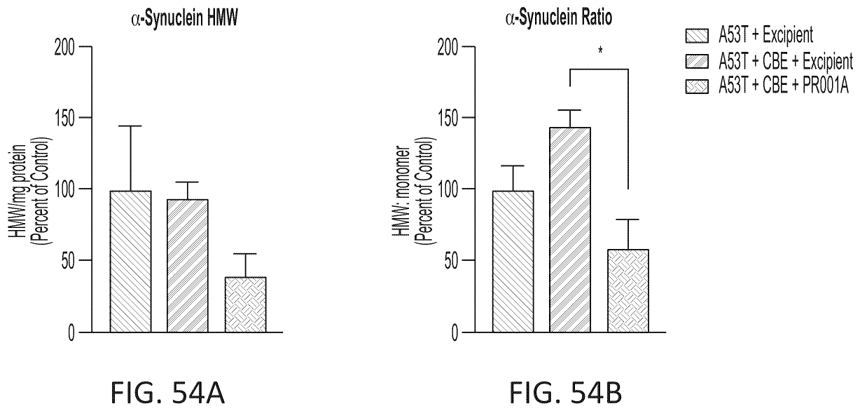

The presence of vector genomes and GCase activity was assessed in the cerebral cortex. For PRV-2018-019, increased cortical glycolipid substrates with CBE treatment were confirmed, and assessed α-Synuclein levels from hippocampal lysates using an automated capillary Simple Western™ immunoblot system on a Jess instrument. Multiple α-Synuclein immunoreactive bands were observed, consistent with the presence of monomers and high molecular weight (HMW) species. A statistically significant reduction in the ratio of HMW α-Synuclein species to monomeric α-Synuclein levels was observed with PR001A treatment of CBE-dosed α-Synuclein transgenic mice (FIG. 54A; FIG. 54B).

### Summary of Nonclinical Efficacy Studies

The studies above show that a single ICV injection of PR001A effectively delivers GBA1 to the CNS and peripheral tissues of mice. In two animal models of PD-GBA and nGD, PR001A elevated GCase activity in the CNS. Increased GCase activity reduced the accumulation of glycolipid substrates in the brain; these glycolipid substrates are proposed as a biomarker outcome measure for the intended clinical trial. Importantly, these benefits persist for at least 6 months after a single treatment with PR001A. The CBE model presents with reactive astrogliosis as well as microgliosis, which are typical histopathological findings in patients with PD-GBA, nGD, and animal models of these disorders (Hamby and Sofroniew, Neurotherapeutics. 2010;7(4):494-506; Farfel-Becker et al., Dis. Model Mech. 2011;4(6):746-752; Farfel-Becker et al., Hum Mol Genet. 2011;20(7):1375-86; Booth et al., Trends Neurosci. 2017;40(6):358-70; McMahon et al., Mol Genet Metab 2018;123(2):593). PR001A is able to prevent or reverse the CBE-induced reactive gliosis and microgliosis. Both models display motor deficits, and treatment with PR001A improves some of these deficits in both models. Alongside these two models, an additional mouse model was used to investigate α-Synuclein pathology. While α-Synuclein phenotypes are variable in mouse models, PR001A was able to suppress or reverse the phenotypes when they were observed; additional in vitro studies support the effectiveness of PR001A in reducing α-Synuclein levels. Together, these studies support the efficacy of PR001A in models of PD-GBA and nGD.

### Example 8.1.4: Toxicology

### Single-Dose Mouse Studies

Safety and toxicology studies conducted with PR001A in mouse models are summarized in Table 15. Two of the mouse model efficacy studies (PRV-2018-005 and PRV-2018-006) also included select safety endpoints such as histopathology to evaluate the safety of PR001A in a disease model.

### Study PRV-2018-005: Dose-Ranging PR001A in CBE Model

Histopathological analysis was performed by hematoxylin and eosin (H&E) staining of the brain, thoracic spinal cord, heart, liver, spleen, lung, and kidney; results were evaluated by a board-certified veterinary pathologist. In the mice treated with CBE, findings in the CNS included glial scars and neuronal necrosis in the cerebral cortex, brain stem, and thoracic spinal cord. Intracerebroventricular PR001A at doses up to 1.3×10¹¹ vg/g was well tolerated in these mice, and this highest dose resulted in a notable reduction in the incidence of these CNS findings; low and mid dose PR001A had a dose-dependent reduction in the number of animals with glial scars in the cerebral cortex, with equivocal effects on the other CNS findings such as neuronal necrosis. No adverse effects of either CBE or PR001A were observed in peripheral tissues. In summary, there were no adverse histopathology findings or evidence of toxicity due to treatment with PR001A in studies with the CBE mouse model.

### Example 9: In Vitro Analysis of rAAV Vectors

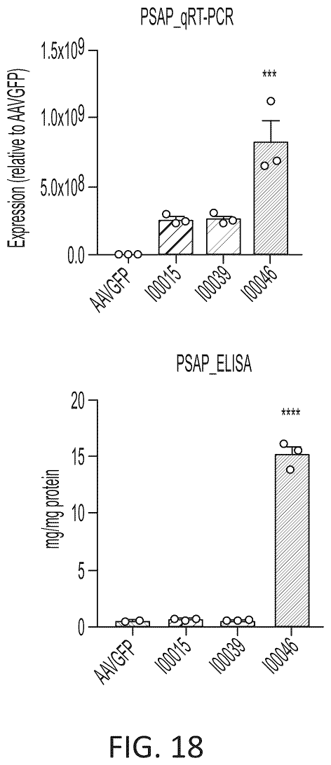

A pilot study was performed to assess in vitro activity of rAAV vectors encoding Prosaposin (PSAP) and SCARB2, alone or in combination with GBA1 and/or one or more inhibitory RNAs. One construct encoding PSAP and progranulin (PGRN) was also tested. Vectors tested include those shown in Table 3. “Opt” refers to a nucleic acid sequence codon optimized for expression in mammalian cells (e.g., human cells). FIG. 18 shows representative data indicating that transfection of HEK293 cells with each of the constructs resulted in overexpression of the corresponding gene product compared to mock transfected cells.

TABLE 3

| | | Inhibitory | | |
|---|---|---|---|---|
| ID | Promoter | RNA | Promoter | Transgene |
| 100015 | JL_intronic | SNCA | JetLong | Opt- |
| | | | | PSAP_GBA1 |
| 100039 | — | — | JetLong | Opt-PSAP-GRN |
| 100046 | — | — | | Opt-PSAP |
| 100014 | JetLong | SNCA | JetLong | Opt- |
| | | | | SCARB2_GBA1 |

### Example 10: ITR “D” Sequence Placement and Cell Transduction

The effect of placement of ITR “D” sequence on cell transduction of rAAV vectors was investigated. HEK 293 cells were transduced with Gcase-encoding rAAVs having 1) wild-type ITRs (e.g., “D” sequences proximal to the transgene insert and distal to the terminus of the ITR) or 2) ITRs with the “D” sequence located on the “outside” of the vector (e.g., “D” sequence located proximal to the terminus of the ITR and distal to the transgene insert), as shown in FIG. 19. Surprisingly, data indicate that rAAVs having the “D” sequence located in the “outside” position retain the ability to be packaged and transduce cells efficiently (FIG. 20).

### Example 11: In Vivo Toxicity Studies

Fifty (50) mice were administered GBA1-encoding rAAVs via a 4 μl intracerebroventricular (ICV) injection on post-natal day 3. All mice received daily intraperitoneal (IP) injections of conduritol B-epoxide (CBE) or PBS, depending on treatment group, from post-natal day 8 to the end of the study. Animals were euthanized 24 hours after their last IP dose. After euthanasia, target tissues were harvested, drop fixed in chilled 4% paraformaldehyde and stored at 4° C., then sent for histopathological processing and evaluation.

Tissues from the forty-two (42) animals euthanized at 38-40 days were trimmed, processed, and embedded in paraffin blocks. They were then sectioned at ˜5 μm, stained with hematoxylin and eosin (H&E) and affixed to slides for evaluation.

There were no histopathologic findings or evidence of toxicity due to treatment with the rAAVs. In the mice treated with conduritol B-epoxide (CBE), there were findings in the central nervous syste m (CNS) that included glial scars and neuronal necrosis in the cerebral cortex, and neuronal necrosis in the brain stem and thoracic spinal cord. High dose rAAV treatment resulted in a notable reduction in the incidence of these CNS findings, while the low and mid dose virus had a dose dependent reduction in the incidence of glial scars in the cerebral cortex, with equivocal effects on the other CNS findings (FIG. 28).

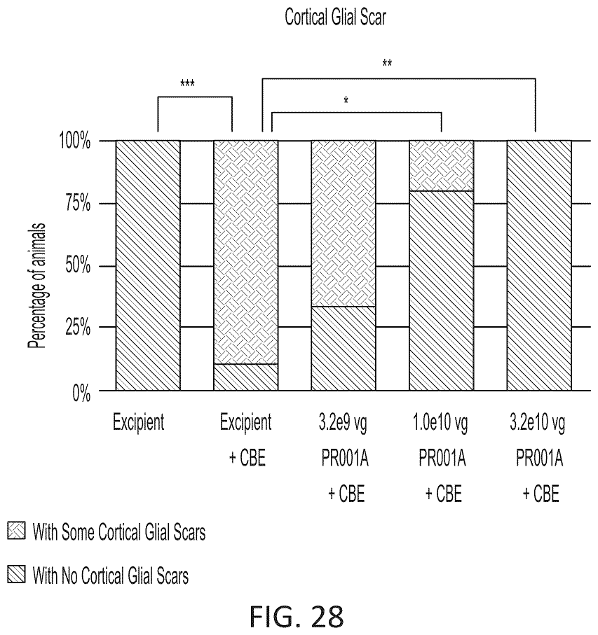

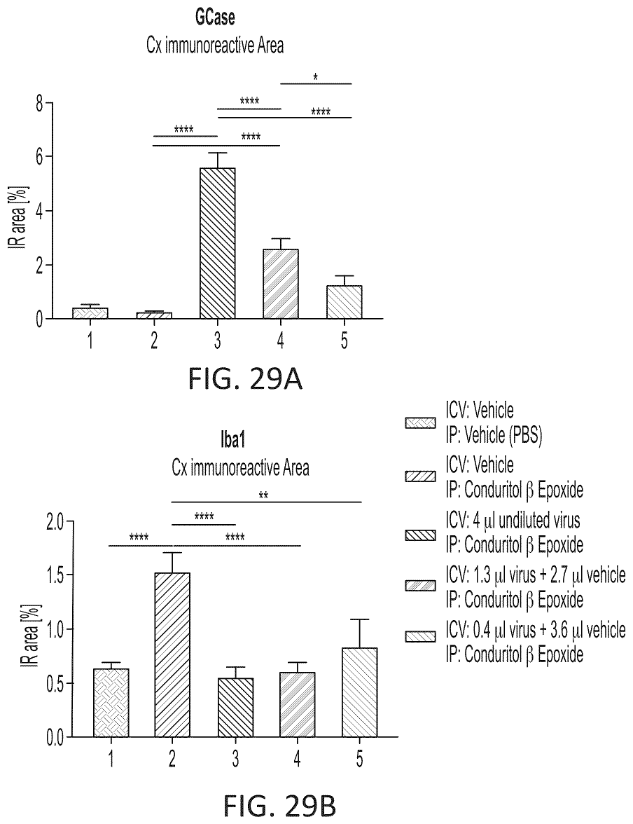

Immunohistochemistry was performed to assess GCase and Iba1 expression in the cortex (FIGS. 29A-29B). GCase expression was significantly increased in all animals treated with rAAV-GBA1 compared to CBE/Excipient treated animals. The increase in GCase expression correlated with the dose delivered, with the highest GCase expression observed in the high-dose treated animals followed by mid- and low-dose treated animals. Iba1, a marker of microgliosis, was significantly increased in animals treated with CBE/Excipient. All doses of rAAV-GBA1 reduced Iba1 staining, thus alleviating microgliosis in the CBE model. Microgliosis is a well described endpoint in neuronopathic GD and models of this disorder.

TABLE 4
Examples of neurodegenerative diseases

| Disease | Associated genes |
|---|---|
| Alzheimer's disease | APP, PSEN1, PSEN2, APOE |
| Parkinson's disease | LRRK2, PARK7, PINK1, PRKN, SNCA, GBA, |
| | UCHL1, ATP13A2, VPS35 |
| Huntington's disease | HTT |
| Amyotrophic lateral sclerosis | ALS2, ANG, ATXN2, C9orf72, CHCHD10, CHMP2B, |
| | DCTN1, ERBB4, FIG4, FUS, HNRNPA1, MATR3, |
| | NEFH, OPTN, PFN1, PRPH, SETX, SIGMAR1, |
| | SMN1, SOD1, SPG11, SQSTM1, TARDBP, TBK1, |
| | TRPM7, TUBA4A, UBQLN2, VAPB, VCP |
| Batten disease | PPT1, TPP1, CLN3, CLN5, CLN6, MFSD8, CLN8, |
| (Neuronal ceroid lipofunscinosis) | CTSD, DNAJC5, CTSF, ATP13A2, GRN, KCTD7 |
| Friedreich's ataxia | FXN |
| Lewy body disease | APOE, GBA, SNCA, SNCB |
| Spinal muscular atrophy | SMN1, SMN2 |
| Multiple sclerosis | CYP2761, HLA-DRB1, IL2RA, IL7R, TNFRSF1A |
| Prion disease (Creutzfeldt-Jakob | PRNP |
| disease, Fatal familial insomnia, | |
| Gertsmann-Straussler-Scheinker | |
| syndrome, Variably protease-sensitive | |
| prionopathy) | |

TABLE 5
Examples of synucleinopathies

| Disease | Associated genes |
|---|---|
| Parkinson's disease | LRRK2, PARK7, PINK1, PRKN, SNCA, |
| | GBA, UCHL1, ATP13A2, VPS35 |
| Dementia with Lewy bodies | APOE, GBA, SNCA, SNCB |
| Multiple system atrophy | COQ2, SNCA |

TABLE 6
Examples of tauopathies

| Disease | Associated genes |
|---|---|
| Alzheimer's disease | APP, PSEN1, PSEN2, APOE |
| Primary age-related tauopathy | MAPT |
| Progressive supranuclear palsy | MAPT |
| Corticobasal degeneration | MAPT, GRN, C9orf72, VCP, |
| | CHMP2B, TARDBP, FUS |
| Frontotemporal dementia | MAPT |
| with parkinsonism-17 | |
| Subacute sclerosing panencephalitis | SCN1A |
| Lytico-Bodig disease | |
| Gangioglioma, gangliocytoma | |
| Meningioangiomatosis | |
| Postencephalitic parkinsonism | |
| Chronic traumatic encephalopathy | |

TABLE 7
Examples of lysosomal storage diseases

| Disease | Associated genes |
|---|---|
| Niemann-Pick disease | NPC1, NPC2, SMPD1 |
| Fabry disease | GLA |
| Krabbe disease | GALC |
| Gaucher disease | GBA |
| Tach-Sachs disease | HEXA |
| Metachromatic leukodystrophy | ARSA, PSAP |
| Farber disease | ASAH1 |
| Galactosialidosis | CTSA |
| Schindler disease | NAGA |
| GM1 gangliosidosis | GLB1 |
| GM2 gangliosidosis | GM2A |
| Sandhoff disease | HEXB |
| Lysosomal acid lipase deficiency | LIPA |
| Multiple sulfatase deficiency | SUMF1 |
| Mucopolysaccharidosis Type I | IDUA |
| Mucopolysaccharidosis Type II | IDS |
| Mucopolysaccharidosis Type III | GNS, HGSNAT, NAGLU, SGSH |
| Mucopolysaccharidosis Type IV | GALNS, GLB1 |
| Mucopolysaccharidosis Type VI | ARSB |
| Mucopolysaccharidosis Type VII | GUSB |
| Mucopolysaccharidosis Type IX | HYAL1 |
| Mucolipidosis Type II | GNPTAB |
| Mucolipidosis Type III alpha/beta | GNPTAB |
| Mucolipidosis Type III gamma | GNPTG |
| Mucolipidosis Type IV | MCOLN1 |
| Neuronal ceroid lipofuscinosis | PPT1, TPP1, CLN3, CLN5, CLN6, |
| | MFSD8, CLN8, CTSD, DNAJC5, |
| | CTSF, ATP13A2, GRN, KCTD7 |
| Alpha-mannosidosis | MAN2B1 |
| Beta-mannosidosis | MANBA |
| Aspartylglucosaminuria | AGA |
| Fucosidosis | FUCA1 |

### Example 12: Non-Human Primate Studies with rAAV Encoding Gcase

The safety of PR001A (AAV9.CBA.GBA1.A), comprising the codon-optimized coding sequence of human GBA1 (SEQ ID NO:15), was evaluated in vivo in non-human primates (NHPs). Additional details of the PR001A components are provided above. The brain of the NHP is most similar to that of humans, and the anatomical features of the NHP spinal cord and CSF volume and flow permits an ICM (intra-cisterna magna) injection. Because of the anatomical similarities to humans, it was expected that NHP studies would provide reliable biodistribution data supporting clinical dosing of PR001A.

Safety and biodistribution of PR001A were evaluated in three toxicology studies in cynomolgus macaques (Table 8): two non-GLP (Good Laboratory Practice) studies (PRV-2018-015 and PRV-2019-005) and a larger 21CFR58 GLP-compliant study (PRV-2018-016).

TABLE 8
Overview of NHP Nonclinical Safety Studies Using PR001A

| | | | | Dose | | | |
|---|---|---|---|---|---|---|---|
| | | | | Groups | Total | Necropsy | |
| Study | Regulatory | Species | | (vg/g | PR001A | Time | |
| number | Oversight | (Age) | ROA | brain) | Dose (vg) | Points | Endpoints |
| PRV- | Non-GLP | Cynomolgus | ICM | 0 | 0 | D18 | In-Life Safety; |
| 2018-015 | | (2-3 years of | ICM + | 2.0 × 10¹¹ | 1.47 × 10¹³ | | Biodistribution; |
| | | age) | IPa | 2.1 × 10¹¹ | 1.53 × 10¹³ | | Histopathology |
| PRV- | GLP | Cynomolgus | ICM | 0 | 0 | D7, D30, | In-Life Safety; |
| 2018-016 | | (2-4 years of | | 6.2 × 10¹⁰ | 4.6 × 10¹² | D183 | Biodistribution; |
| | | age) | | 2.3 × 10¹¹ | 1.7 × 10¹³ | | Histopathology; |
| | | | | | | | CBC; Vector |
| | | | | | | | Shedding |
| PRV- | Non-GLP | Cynomolgus | ICM | 0 | 0 | D30, D90 | In-Life Safety; |
| 2019-005 | | (2-3 years of | | 7.0 × 10¹¹ | 5.2 × 10¹³ | | Biodistribution; |
| | | age) | | | | | Histopathology |

Abbreviations:
CBC, complete blood count;
D, day;
GLP, Good Laboratory Practice;
ICM; intra-cisterna magna;
IPa, intraparenchymal;
NHP, nonhuman primate;
ROA, route of administration;
vg, vector genome.

A pilot non-GLP study (PRV-2018-015) was conducted in NHPs to confirm that the final PR001A product is delivered to the NHP brain following ICM administration. The GLP toxicology and biodistribution study in NHPs (PRV-2018-016) assessed the safety and biodistribution of PR001A.

The doses tested in NHPs include the maximum feasible dose as determined by the volume administered and test product titer. In addition, a lower dose was also evaluated in the GLP study. The time points of the GLP study were selected to evaluate safety after treatment but before peak expression (Day 7), the start of peak expression (Day 30), and long-term expression post peak (Day 183).

### Study PRV-20I8-015: Non-GLP NHP Study of PR001A

A non-GLP pilot tolerance and biodistribution study of PR001A was conducted in male cynomolgus monkeys. The goal of this study was to verify biodistribution of PR001A to various brain areas and major peripheral organs following ICM delivery. The time point for sacrifice was selected because it was predicted to allow for a meaningful measure of potential early toxicity to inform the planned GLP NHP toxicology study, most notably with early in-life observations as measured by a functional observational battery (FOB). Studies of intrathecal AAV delivery have demonstrated that transgene expression peaks 2 to 3 weeks after injection (Hinderer et al., Mol Ther. 2014;22(12):2018-27; Hinderer et al., Mol Ther Methods Clin Dev. 2014;1:14051; Hinderer et al., Mol Ther. 2015;23(8)1298-307; Hinderer et al., Mol Genet Metab. 2016;119(1-2):124-30). Day 18 evaluations, therefore, should detect immediate toxicity due to the injection procedure or an innate inflammatory response to the test article, as well as provide information regarding transgene biodistribution and expression at a time point corresponding to early peak expression. The study design included an arm with rapamycin treatment (0.3 mg/kg oral, Day −3 to Day 18) in combination with PR001A to determine if immunosuppression would be beneficial in mitigating potential toxicity. To increase transgene expression in the brain, one arm in the study included intraparenchymal (IPa) administration of PR001A directly into the midbrain targeting bilateral substantia nigra pars compacta in combination with ICM delivery. The ICM dose volume was 0.5 mL, the maximum volume there was experience with administering, and the IPa dose was 10 μL bilateral, translating to doses of 1.47×10¹³ vg for ICM alone and 1.53×10¹³ vg for treatment with both ICM and IPa. With an estimated brain weight of 74 g, this translates to an ICM dose of 2.0×10¹¹ vg/g brain and a dose of 2.1×10¹¹ vg/g brain for the group receiving ICM administration in combination with IPa. A tabulated summary of this study's design is provided in Table 9.

TABLE 9
Overview of the Non-GLP NHP Study PRV-2018-015
Biodistribution and Safety Study Following PR001A Administration in NHPs

| Purpose | Assess the tolerance and biodistribution of PR001A in | | |
|---|---|---|---|
| | NHPs | | |
| Regulatory Compliance | Non-GLP | | |
| Test Article | PR001A | | |
| Total No. of Animals | 8 male cynomolgus monkeys | | |
| Weight (age) | 3-4 kg (2-3 years) | | |
| Number of Animals/Group | 2/group | | |
| Study Design | Group Assignments (see Table 9A) | | |
| Dosing Route and | ICM using a syringe; single injection of 0.5 mL IPa using | | |
| Frequency | Hamilton syringe; bilateral injection of 10 μL to each | | |
| | hemisphere | | |
| Formulations | Dosing solution provided at concentration of 2.9 × 10¹³ | | |
| | vg/mL; excipient used in the control group is a similar | | |
| | formulation as intended for the clinic (20 mM Tris pH 8.0, | | |
| | 200 mM NaCl, 1 mM MgCl₂, and 0.001% [w/v] poloxamer | | |
| | 188) | | |
| FOB | Weekly | | |
| Body Weights | Weekly | | |
| Necropsy | Day 18 | | |
| H&E and qPCR | The following tissues were examined from all animals in all | | |
| | groups: | | |
| | liver | frontal cortex | paraventricular |
| | lung | parietal cortex | nucleus |
| | kidney | occipital cortex | pons |
| | gonads | insular cortex | entorhinal cortex |
| | heart | cingulate cortex | medulla |
| | spleen | hippocampus | cerebellum dorsal |
| | root ganglia | putamen | midbrain cervical |
| | | | spinal cord |

For midbrain H&E, includes at least 12 sections that include
6 sections around the infusion site in the IPa group; the ICM
alone groups (1-3) include the same anatomical levels

Abbreviations:
FOB, functional observational battery;
GLP, Good Laboratory Practice;
H&E, hematoxylin and eosin;
ICM; intra-cisterna magna;
Immunosupp, immunosuppressed;
IPa, intraparenchymal;
MgCl₂; magnesium chloride;
NaCl, sodium chloride;
NHP, nonhuman primate;
qPCR, quantitative polymerase chain reaction;
ROA, route of administration;
vg, vector genome(s).

TABLE 9A
Group Assignments

| | Dose | | | Number of |
|---|---|---|---|---|
| Group | (vg/g brain) | ROA | Immunosupp. | Animals |
| 1 | 0 | ICM | No | 2 |
| 2 | 2.0 × 10¹¹ | ICM | No | 2 |
| 3 | 2.0 × 10¹¹ | ICM | Yes | 2 |
| 4 | 2.1 × 10¹¹ | ICM + IPa | No | 2 |

The H&E analysis was performed by two independent board-certified veterinary pathologists, and both concluded there were no PR001A-related toxicity findings. Spinal cord changes observed were likely the result of trauma at the time of ICM injection and were not considered related to PR001A. All histopathology findings in non-nervous system tissue were considered spontaneous or incidental changes commonly seen in control monkeys. Overall, there were no definitive adverse PR001A effects in the brain or spinal cord.

The reviewing pathologist noted nonspecific changes (predominantly variable infiltrates of mononuclear cells) in the meninges, brain or spinal cord parenchyma, and/or at the injection site (in these tissues) were likely associated with the test article, but the pathologist did not consider these changes to be adverse. At the severities noted, similar infiltrates might reasonably be expected to be observed in any monkey with an experimental procedure that disrupts the meninges and/or the blood brain barrier. Additionally, some infiltrates (notably those within the choroid plexus and occasionally in the parenchyma) are commonly observed in control monkeys (Butt et al., Toxicol Pathol. 2015;43:513-8). All other histopathologic findings observed were considered incidental and/or were of similar incidence and severity in excipient and PR001A-treated animals and, therefore, were considered unrelated to administration of PR001A. A second independent, board-certified veterinary pathologist reviewing the same tissue samples noted that all findings were indistinguishable from incidental findings or trauma incurred during the injection procedure as findings were nonspecific and across all groups, including the control group receiving only excipient. In addition, a different board-certified veterinary pathologist reviewed the non-GLP tissues and concluded there were no PR001A-related effects.

Overall, there were no changes in FOB scores, body weight gain, or food consumption during the course of the study irrespective of group and across time points. Microglia morphology in the midbrain did not appear to differ across treatment groups (as determined with Iba1 staining). Expression and morphology of tyrosine hydroxylase positive neurons of the midbrain did not appear to differ across treatment groups. By Day 18, AAV9-nAb titers were increased in all PR001A-treated animals, while the excipient-treated control animals showed only modest changes compared to baseline. One of the monkeys in the group receiving oral rapamycin had a lower AAV9 nAb titer (1:64) at Day 18 compared to the other animals receiving PR001A treatment (>1:256); the difference in titers did not appear to affect biodistribution, but the sample size is too low to be conclusive.

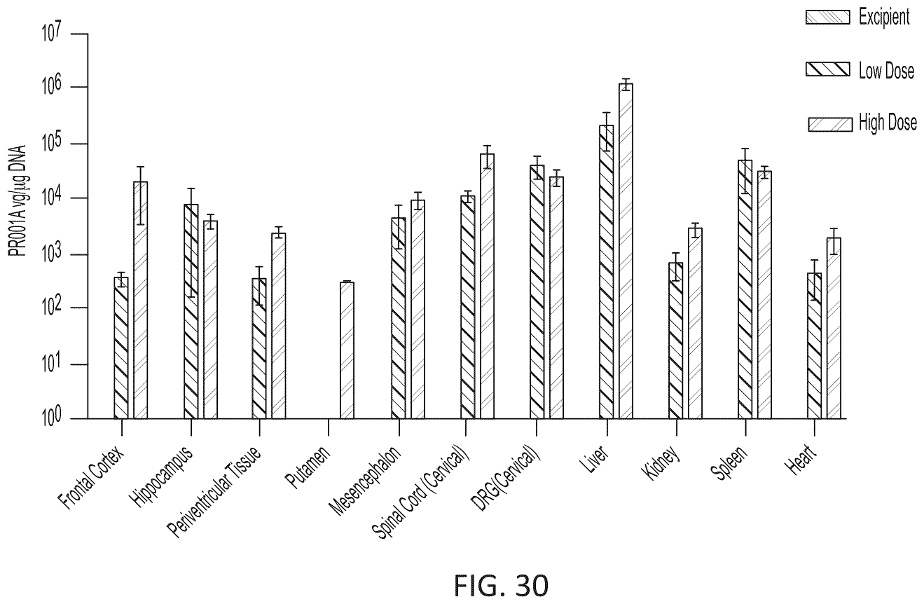

Biodistribution was evaluated in all test samples collected using quantitative polymerase chain reaction (qPCR); tissues were considered positive with at least 100 vg/ρg DNA (these criteria were also used to assess positive tissues in the mouse efficacy studies). All tissues tested were positive in all groups that were treated with PR001A, indicating widespread distribution throughout the CNS and periphery. In addition, animals that received ICM administration of PR001A in combination with bilateral IPa administration into the midbrain had increased localized expression. Treatment with rapamycin did not appear to have any effect on safety or biodistribution (select representative regions shown in FIG. 30). Of note, several of the tissues from control animals (pons, spinal cord, paraventricular, dorsal root ganglia, and lung) were also positive as determined by qPCR. Several issues were noted about the necropsy procedures that indicated an increased risk for cross-contamination across animals in different treatment groups and between different organs within each animal. Changes were implemented in the necropsy procedure to minimize contamination for future studies. However, there were no adverse toxicity findings in any of the animals that were positive for qPCR. Analysis of the transgene expression (GCase activity) indicated no significant increase in GCase activity in the PR001A-treated animals compared to controls; GCase activity was explored in more detail in the GLP NHP toxicity Study PRV-2018-016.

Taken together, the results of non-GLP NHP Study PRV-2018-015 indicated no safety or toxicity concerns with any of the in-life or postmortem assessments. All animals survived until their scheduled necropsy date, and postmortem pathology analysis indicated no adverse toxicity concerns. The study also showed uniform biodistribution of PR001A in the brain.

### Study PRV-2018-016: A GLP NHP Study of PR001A

### Study Design

The purpose of this GLP study was to evaluate the toxicity and biodistribution of PR001A when administered once via ICM injection in cynomolgus monkeys with a 7-, 30-, or 183-day post-administration observation period. The study was designed to evaluate 2 dose levels: the highest dose is the maximum feasible dose achievable with 1.2 mL volume (the highest volume there was experience with administering) of undiluted test product, and a lower dose ½ log unit lower than the high dose. The doses equated to a low dose of 4.6×10¹² vg and a high dose of 1.7×10¹³ vg; with a brain weight estimate of 74 g in a cynomolgus monkey, this translates to approximately 6.2×10¹⁰ vg/g brain and 2.3×10¹¹ vg/g brain. The study also included a control arm in which animals receive 1.2 mL of excipient only (20 mM Tris pH 8.0, 200 mM NaCl, 1 mM MgCl₂, and 0.001% [w/v] poloxamer 188). This study utilized both male and female cynomolgus macaques. The Day 7 group included 1 male at the highest dose and was designed as a sentinel for early toxicity; the remaining 2 time points (Day 30 and Day 183) included 2 males and 1 female at each dose. In addition to samples from multiple brain regions, peripheral tissue samples were collected for qPCR analysis. All samples that were positive with qPCR were analyzed for transgene expression. A tabulated summary of this study's design is provided in Table 10.

TABLE 10
Overview of the GLP NHP Study PRV-2018-016
A Single-dose Intra-cisternal Toxicity and Biodistribution Study in Cynomolgus Monkeys
with a 7-day, 30-day, or 183-day Observation Period

| Purpose | Assess the tolerance and biodistribution of PR001A in NHPs | |
|---|---|---|
| Regulatory Compliance | GLP | |
| Test Article | PR001A | |
| Total No. of Animals | 19 cynomolgus monkeys | |
| Weight (age) | 2-5 kg (25-50 months) | |
| Study Design | Group Assignments (see Table 10A) | |
| Dosing Route and Frequency | ICM using a syringe; 1-3 cc syringe and spinal needle | |
| | (Pencan 25 G × 2.5 cm BBraun); single slow bolus delivered | |
| | at a maximum rate of 0.5 cc/min | |
| Formulations | Dosing solution provided at concentration of 1.42 × 10¹³ | |
| | vg/mL | |
| Clinical Signs | Daily (including food consumption); detailed observations | |
| | weekly | |
| Body weights | Weekly | |
| Neurological, Ophthalmic, | Once pre-dose and during Weeks 2 and 26 | |
| and Electrocardiogram | | |
| Examinations | | |
| Clinical Pathology | All groups hematology, clinical chemistry, coagulation | |
| | parameters | |
| Hematology | red blood cell count | mean corpuscular volume |
| | hemoglobin | platelet count |
| | hematocrit | white blood cell count |
| | mean corpuscular | blood smear |
| | hemoglobin | absolute reticulocyte count |
| | mean corpuscular | leukocyte count |
| | hemoglobin concentration | differential blood cell count |
| Clinical Chemistry | glucose | alanine aminotransferase |
| | urea nitrogen | alkaline phosphatase |
| | creatinine | gamma glutamyltransferase |
| | total protein | aspartate aminotransferase |
| | albumin | calcium |
| | globulin | inorganic phosphorus |
| | albumin/globulin ratio | sodium |
| | cholesterol | potassium |
| | total bilirubin | chloride |
| | creatine kinase | triglycerides |
| Coagulation | prothrombin time | |
| | fibrinogen | |
| | activated partial thromboplastin time | |
| Vector Shedding (urine/feces) | At sacrifice | |
| Necropsy | Day 7, Day 30, Day 183 | |
| Tissue Preservation for | The following tissues from each animal will be collected in | |
| Histopathology | 10% neutral-buffered formalin (unless otherwise indicated) | |
| | or recorded as missing, if applicable: | |
| Histopathology | All groups - all tissues | |
| Biodistribution | The following tissues will be analyzed for biodistribution by | |
| | qPCR: | |
| | Frontal cortex | Liver |
| | Hippocampus | DRG (cervical) |
| | Ventral mesencephalon | DRG (thoracic) |
| | Periventricular gray | DRG (lumbar) |
| | Putamen | Spinal cord (thoracic) |
| | Testis | Spinal cord (lumbar) |
| | Ovary | Spinal cord (cervical) |
| | Kidney | Spleen |
| | Stomach (pyloric) | Heart (apex) |
| | Blood | CSF |

| GCase Expression | All samples that are positive for qPCR will be evaluated for | | |
|---|---|---|---|
| | GCase expression | | |
| Tissue Preservation | Adrenalª | Injection site | Rectum |
| | Aorta | (overlying skin) | Salivary gland |
| | Bone, femur with | Jejunum | Sciatic nerve |
| | Bone marrow | Kidneyª | Seminal vesicle^{a} |
| | with bone marrow | Liverª | Spinal cord |
| | Brainª | Lung with large | (cervical, |
| | thoracic, | bronchi | lumbar) |
| | Cecum | Lymph node | Spleenª |
| | Cervix | (mandibular) | Stomach |
| | Colon | Lymph node | Testis^{a} |
| | Duodenum | (mesenteric) | Thymus^{a} |
| | Epididymisª | Mammary gland | Thyroid with |
| | Esophagus | Muscle, biceps | parathyroidª |
| | Eye^{b} | femoris | Tongue |
| | Gall bladder | Optic nerve | Trachea |
| | GALT (Peyer's | Ovaryª | Urinary bladder |
| | patch) | Oviducts | Uterusª |
| | Heartª | Pancreas | Vagina |
| | Ileum | Pituitary gland | |
| | | Prostrateª | |

^{a}Organs (when present) will be weighed or noted as missing:
^{b}Collected in modified Davidson's fixative and stored in10% neutral buffered formalin
Abbreviations:
CSF, cerebrospinal fluid;
DRG, dorsal root ganglia;
F, female;
GALT, gut-associated lymphoid tissue;
GLP, Good Laboratory Practice;
ICM; intra-cisterna magna;
M, male;
MgCl₂; magnesium chloride;
NaCl, sodium chloride;
NHP, nonhuman primate;
qPCR, quantitative polymerase chain reaction;
vg, vector genome(s).ª
20 mM Tris pH 8.0, 200 mM NaCl, 1 mM MgCl₂, and 0.001% (w/v) poloxamer 188.

TABLE 10A
Group Assignments

| | | Number of animals | | |
|---|---|---|---|---|
| | Dose | Necropsy | Necropsy | Necropsy |
| Group | (vg/g brain) | (Day 7) | (Day 30) | (Day 183) |
| 1 | 0 | 0 | 2M/1F | 2M/1F |
| 2 | 6.2 × 10¹⁰ | 0 | 2M/1F | 2M/1F |
| 3 | 2.3 × 10¹¹ | IM | 2M/1F | 2M/1F |

Cynomolgus NHPs were assessed by multiple in-life observations and measurements, including mortality/morbidity (daily), clinical observations (daily), body weight (baseline and weekly thereafter), visual inspection of food consumption (daily), neurological observations (baseline and during Weeks 2 and 26), indirect ophthalmoscopy (baseline and during Weeks 2 and 26), and electrocardiographic measurement (baseline and during Weeks 2 and 26).

Analysis of nAb to the AAV9 capsid was performed at baseline and at sacrifice on Days 7, 30, or 183. Clinical pathology consisting of hematology, coagulation, clinical chemistry, and urinalysis was performed twice at baseline (blood tests; once for urinalysis) and once during Weeks 1 and 13 of the dosing phase.

Animals were euthanized, and tissues harvested on Days 7, 30, or 183. The tissues were collected from all animals, weighed (if applicable), and divided into replicates. One replicate was preserved in 10% neutral-buffered formalin (except when special fixatives are required for optimum fixation) for histopathological evaluation (all animals). Additional replicates were collected for qPCR and transgene expression analysis.

### Safety and Toxicity

All animals survived to the scheduled necropsy date with no unexpected deaths. There were no concerns or issues with the in-life assessments for any of the groups; gross macroscopic examination at necropsy showed no PR001A-related abnormalities in any of the cohorts.

No PR001A-related organ weight differences or macroscopic or microscopic findings were present in any of the groups at the interim sacrifices on Day 7 or 30 or at the terminal sacrifice on Day 183. Hemorrhage, characterized by focal areas of perivascular hemorrhage mainly in region of the brain stem, was present across all groups including controls, and, therefore, was considered procedure-related (CSF collection prior to necropsy) and not related to PR001A. All other microscopic findings, including minimal mononuclear infiltrates in the brain or spinal cord, were considered spontaneous and/or incidental because they occurred at a low incidence, were randomly distributed across groups (including concurrent controls), and/or their severity was as expected for monkeys of this age; therefore, they were considered not related to PR001A.

No PR001A-related findings were observed in clinical pathology test results; increased fibrinogen was noted in the animal exhibiting the highest anti-AAV9 titer consistent with an immune response against the vector. Positive titers for anti-AAV9 antibodies were observed by Day 7 in all animals administered PR001A. No PR001A-related clinical observations, body weight changes, ophthalmic observations, or physical or neurological examination findings were noted. No PR001A-related differences in mean PR interval, QRS duration, QT interval, corrected QT (QTc) interval, or heart rate were observed in males only or combined sexes administered either dose of PR001A. No PR001A-related arrhythmias or abnormal waveforms were observed.

Dose levels of 0, 6.2×10^{10 ,} or 2.3×10¹¹ vg/g brain PR001A were well tolerated when administered via single injection at the cisterna magna to male and female monkeys. No in-life, clinical pathology, or anatomic pathology observations were observed that were considered related to the gene product in PR001A.

### Biodistribution and Immune Response

Biodistribution analysis of vector genome copies was performed using a qPCR-based assay (vector presence); expression of the transgene (GBA1) was measured in samples that were positive for vector genome presence. At Days 30 and 183, all tissues examined (including CNS and peripheral) were positive by qPCR analysis following treatment with the high dose (2.3×10¹¹ vg/g brain) (select representative regions from Day 183 shown in FIG. 30). At Day 30, tissue samples collected from the testes and ovaries were positive for transduction in all NHPs treated with the high dose of PR001A (2.3×10¹¹ vg/g brain). In addition, 1 male NHP treated with the low dose of PR001A (6.2×10¹⁰ vg/g brain) was positive in the testes at Day 30. At Day 183, 1 male and 1 female were positive for PR001A transduction in the gonads after treatment with the high dose, and 2 males treated with the low dose were positive in the testes.

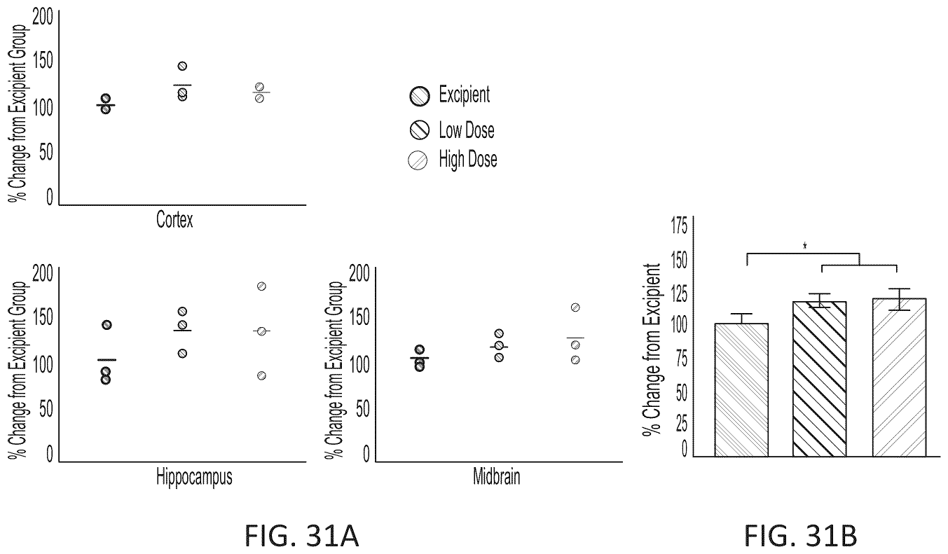

To confirm that human GCase was produced in the treated NHPs, protein levels were evaluated on a Simple WesternTM immunoblot system on a Jess instrument. Results from cortex, hippocampus, and midbrain samples obtained from NHPs dosed with PR001A indicated elevated levels of GCase expression when analyzed in aggregate compared to the samples from normal NHPs that only received excipient; both low dose and high dose groups were combined for statistical comparison to the control group (FIG. 31A and FIG. 31B). These results indicate that the effective and broad transduction of PR001A in NHPs following ICM administration lead to increased GCase expression.

In conclusion, the biodistribution findings indicate that ICM administration of PR001A in NHPs results in robust and broad transduction of the human GBA1 transgene in the brain and peripheral organs. In summary of the NHP biodistribution data, ICM administration of PR001A results in broad biodistribution throughout the brain comparable to levels shown to be efficacious in the mouse models; this transduction leads to the elevation of GCase protein levels in the brain.

### Study PRV-2019-005: Non-GLP NHP Study of PR001A

### Study Design

A non-GLP study was conducted in 12 male cynomolgus macaques to evaluate toxicity and biodistribution of PR001A when administered once via ICM injection with a 30- and 90-day post-administration observation period. The study was designed to evaluate a single dose level: 5.2×10¹³ vg, or 7.0×10¹¹ vg/g brain assuming an average brain weight of 74 g in cynomolgus macaques. The dose administered is the maximum feasible dose achievable with 1.2 mL volume (the highest volume there was experience with administering) of undiluted PR001A product. The study included a control arm in which animals receive 1.2 mL of excipient only (20 mM Tris pH 8.0, 200 mM NaCl, and 1 mM MgCl2+0.001% [w/v] Pluronic F68). Samples from multiple brain regions and peripheral organs were collected for qPCR analysis to measure biodistribution, and clinical pathology measurements and histopathology were performed to evaluate safety. A tabulated summary of this study's design is provided in Table 11.

TABLE 11
Overview of the Non-GLP NHP Study PRV-2019-005
Non-GLP toxicology and biodistribution study following intra-cisterna magna PR001A
administration in non-human

| Purpose | Assess the tolerance and biodistribution of PR001A in | |
|---|---|---|
| | NHPs | |
| Regulatory Compliance | Non-GLP | |
| Test Article | PR001A | |
| Total No. of Animals | 12 cynomolgus monkeys | |
| Weight (age) | 2-4 kg (2-3 years) | |
| Study Design | Group Assignments (see Table 11A) | |
| Dosing Route and Frequency | Intra-cisterna magna; single slow bolus delivered over 3 | |
| | minutes | |
| Formulations | Dosing solution provided at concentration of 4.3 × 10¹³ | |
| | vg/mL | |
| Clinical Signs | Daily (including food consumption) | |
| Body Weights | Daily | |
| FOB | Days −14, 7, 30, 60, 90 | |
| Clinical Pathology (chemistry | Days −14, 7, 30, 60, 90 | |
| and hematology) | | |
| Hematology | red blood cell count | mean corpuscular volume |
| | hemoglobin | platelet count |
| | hematocrit | white blood cell count |
| | mean corpuscular | blood smear |
| | hemoglobin | reticulocyte count |
| | mean corpuscular | differential blood cell count |
| | hemoglobin concentration | |
| Clinical Chemistry | glucose | alanine aminotransferase |
| | alkaline | phosphatase |
| | urea nitrogen | gamma glutamyltrasnferase |
| | creatinine | aspartate aminotransferase |
| | total protein | calcium |
| | albumin | inorganic phosphorus |
| | globulin | sodium |
| | albumin/globulin ratio | potassium |
| | cholesterol | chloride |
| | total bilirubin | triglycerides |
| Necropsy | Day 30 and Day 90 | |
| Tissue Preservation | The following tissues were examined from all animals in all | |
| | groups: | |
| | frontal cortex | liver |
| | hippocampus | kidney |
| | ventral mesencephalon | heart (apex) |
| | periventricular nucleus | spleen |
| | putamen | stomach (pyloric) |
| | dorsal root ganglion (cervical) | testes |
| | dorsal root ganglion (thoracis) | spinal cord (cervical) |
| | dorsal root ganglion (lumbar) | spinal cord (thoracic) |
| | CSF | spinal cord (lumbar) |

| | |
|---|---|
| Histopathology | Samples from the tissue list above will be preserved and |
| | paraffin wax embedded for H&E histology |
| Biodistribution | The tissue list shown above will be collected and stored. |
| | Samples from all animals will be analyzed for |
| | biodistribution by quantitative PCR (qPCR). |

Abbreviations:
CSF, cerebrospinal fluid;
FOB, functional observational battery;
GLP, Good Laboratory Practice;
H&E, hematoxylin and eosin;
NHP, nonhuman primate;
qPCR, quantitative polymerase chain reaction;
vg, vector genome(s).

TABLE 11A
Group Assignments

| | Dose | Number of Animals | |
|---|---|---|---|
| Group | (vg/g brain) | Day 30 | Day 90 |
| 1 | 0 | 3 | 3 |
| 2 | 7.0 × 10¹¹ | 3 | 3 |

As part of this study, tissues were fixed in 10% formalin, embedded in paraffin, and processed to produce H&E-stained slides. Digital slides were prepared and examined by an independent board-certified veterinary pathologist. At both 30 and 90 days post treatment, there were no findings attributed to treatment with PR001A as findings in the PR001A-treated animals were either consistent with those commonly observed in cynomolgus macaque monkeys (Chamanza et al., Toxicol Pathol. 2010;38(4):642-57), and/or were observed in both vehicle control animals and animals treated with PR001A, and, therefore, were considered incidental.

There was no effect of PR001A, administered to the cisterna magna, on weight gain or food consumption as there was no statistical difference between the treatment and control groups during the course of the study. In addition, there was no change in FOB scores irrespective of group and across timepoints, indicating no issues or concerns during the in-life phase of the study. Plasma levels of nAb against AAV9 were measured using an in vitro assay. Samples were prepared from animals in the study at baseline (pre-ICM administration) and at time of sacrifice (either Day 30 or 90). Treatment with PR001A resulted in increases in AAV9 nAb titers between baseline and time of necropsy at both Days 30 and 90, while vehicle-treated animals' titers overall remained stable or decreased.

### Biodistribution and Expression of PR001A

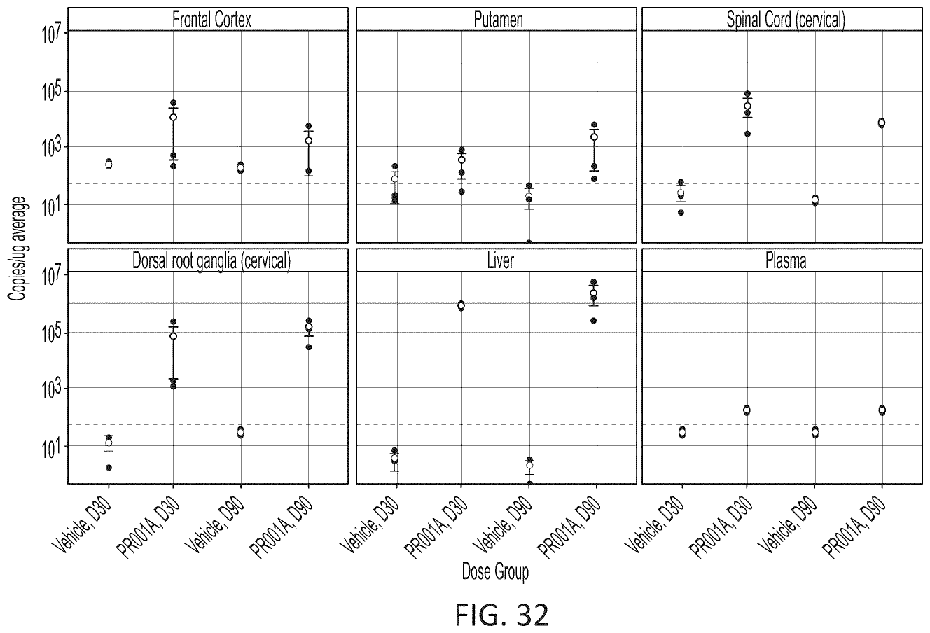

Biodistribution of the PR001A transgene was evaluated in all test samples collected using qPCR; tissues were considered positive with at least 50 vg/μg DNA, the lower limit of quantitation for the assay. All tissues tested were positive in all groups that were treated with PR001A, indicating widespread distribution throughout the CNS and periphery. Data from select representative regions from both the Day 30 and Day 90 cohorts are shown in FIG. 32.

Taken together, the results of non-GLP NHP Study PRV-2019-005 indicate no safety or toxicity concerns with any of the in-life or post-mortem assessments. All animals survived until their scheduled necropsy date, and post-mortem pathology analysis indicated no adverse toxicity concerns.

Safety and toxicology studies conducted with PR001A in NHPs are summarized in Table 16.

### Example 13: Phase 1/2 Trials in Human Subjects

Parkinson's Disease with GBA1 Mutation

Human subjects will be enrolled in a randomized, double-blind, sham procedure-controlled trial of the PR001A rAAV. The subject inclusion criteria comprise: single or biallelic GBA1 mutations, moderate to severe Parkinson's disease, and has stable use of background Parkinson's disease medications prior to investigational product dosing. The subjects will be divided into two groups: (1) PR001 Low Dose (1×10¹⁴ vg) vs. Placebo (N=8, 6:2); and (2) PR001 High Dose (2×10¹⁴ vg) vs. Placebo (N=8, 6:2). Each subject will receive the investigational product as a single ICM (intra-cisterna magna) injection. The trial will include a 3-month biomarker readout, a 12-month clinical readout and a 5-year safety and clinical follow-up. The trial will analyze: (1) safety and tolerability; (2) key biomarkers, including: Gcase, GluCer, and GluSph (CSF and blood); (3) additional biomarkers, including: α-Synuclein, NfL (neurofilament light), DAT (Dopamine transporter) SPECT (single photon emission computed tomography); and MRI (magnetic resonance imaging); and (4) Efficacy: MDS-UPDRS (Movement Disorders Society Unified Parkinson's disease Rating Scale); cognition; and ADLs (Activities of Daily Living).

### Type 2 Gaucher Disease

Human subjects (n=15) will be enrolled in an open-label trial of the PR001A rAAV. The subject inclusion criteria comprise: infants 0-24 months old; biallelic GBA1 mutations; neurological signs and symptoms consistent with Type 2 Gaucher disease; and stable standard of care background medications. Each subject will receive the investigational product as a single ICM (intra-cisterna magna) injection. The trial will include a 3-month biomarker readout, a 12-month clinical readout and a 5-year safety and clinical follow-up. The trial will analyze: (1) safety and tolerability; (2) key biomarkers, including: Gcase, GluCer, and GluSph (CSF and blood); (3) time to clinical event (e.g., tracheostomy, PEG (percutaneous endoscopic gastrostomy) placement, death); and (4) Efficacy: behavior, cognition, gross motor, function, QoL (quality of life).

### Example 14: Studies of Intravenous Administration of rAAV Encoding Gcase

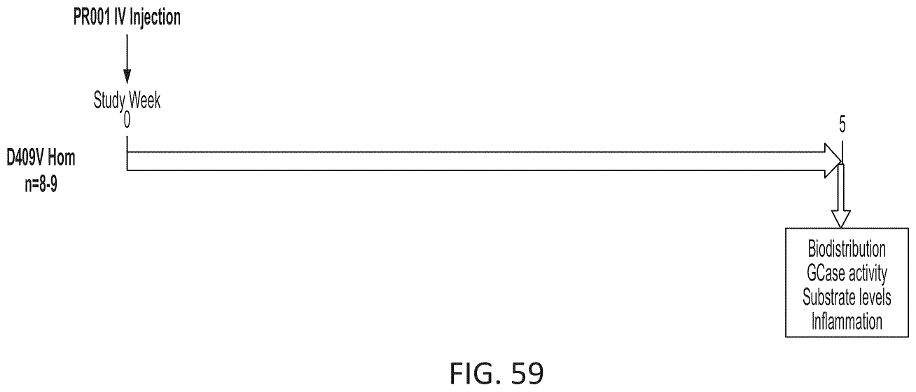

A PR001 intravenous dose ranging study was carried out in the D409V Hom mouse model. Homozygous Gba1^{D409V/D409V} (D409V Hom) mice (The Jackson Laboratory, Bar Harbor, ME) display Gaucher disease-related phenotypes including decreased GCase activity (see, e.g., Sardi et al., Proc Natl Acad Sci USA. 2011;108(29):12101-6). The study design is provided in FIG. 59. The groups and doses are provided in Table 12.

TABLE 12
Groups and doses for study of PR001 intravenous administration
in D409V Hom mice

| Group | Vector genomes/kg |
|---|---|
| WT† + Excipient | N/A |
| D409V + Excipient | N/A |
| D409V + PR001 Dose 1 | 1.1 × 10¹⁰ |
| D409V + PR001 Dose 2 | 1.1 × 10¹¹ |
| D409V + PR001 Dose 3 | 1.1 × 10¹² |
| D409V + PR001 Dose 4 | 1.1 × 10¹³ |
| D409V + PR001 Dose 5 | 1.1 × 10¹⁴ |

†Wild type animals purchased from The Jackson Laboratory (Bar Harbor, ME), not littermates.

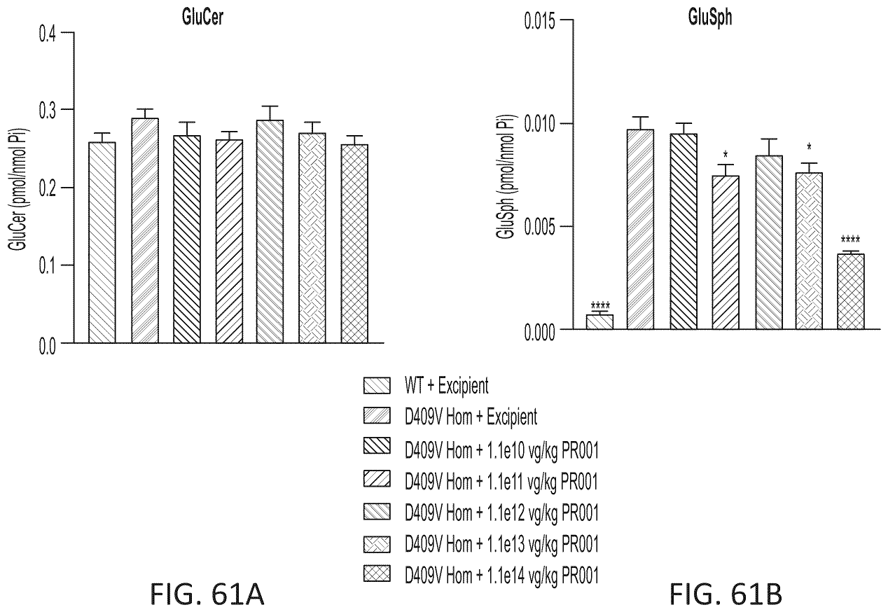

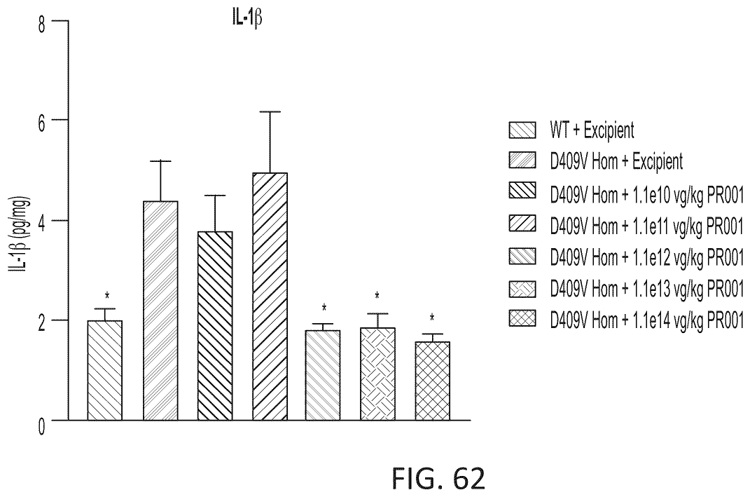

Intravenous administration of PR001 decreased inflammation in the liver (FIG. 60A). D409V Hom mice showed glycolipid accumulation in the liver which was suppressed in a dose-dependent manner by PR001 treatment (FIG. 60B; FIG. 60C). D409V Hom mice showed GluSph accumulation in the brain, which was decreased by PR001 treatment (FIG. 61B). Intravenous administration of PR001 decreased inflammation in the lung (FIG. 62).

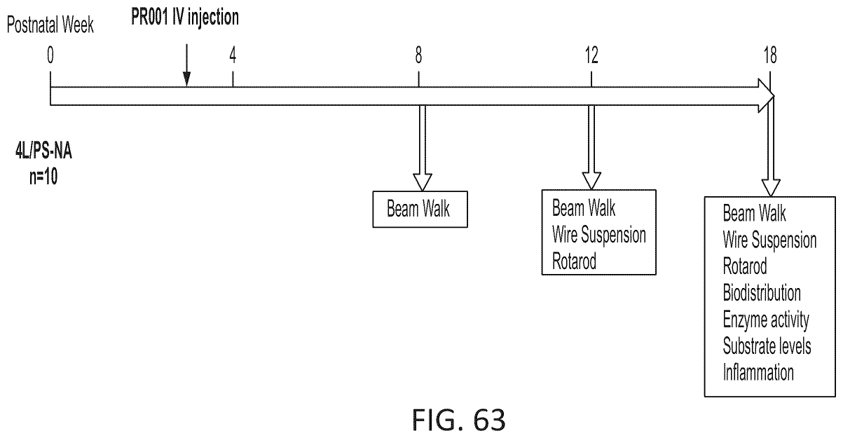

A PR001 intravenous dose ranging study was also carried out in the 4L/PS-NA mouse model. The study design is provided in FIG. 63. The groups and doses are provided in Table 13.

TABLE 13
Groups and doses for study of PR001 intravenous administration
in 4L/PS-NA mice

| Group | Vector genomes/kg |
|---|---|
| Control + Excipient | N/A |
| 4L/PS-NA + Excipient | N/A |
| 4L/PS-NA + PR001 Dose 1 | 9.5 × 10¹² |
| 4L/PS-NA + PR001 Dose 2 | 3.0 × 10¹³ |
| 4L/PS-NA + PR001 Dose 3 | 9.5 × 10¹³ |
| 4L/PS-NA + PR001 Dose 4 | 3.0 × 10¹⁴ |

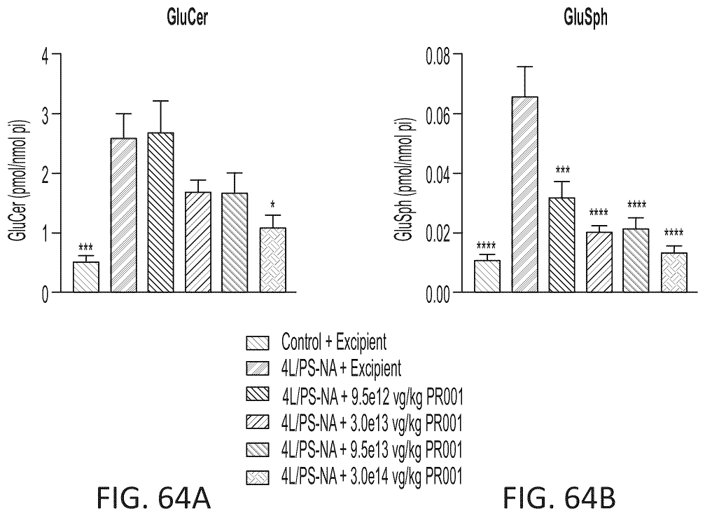

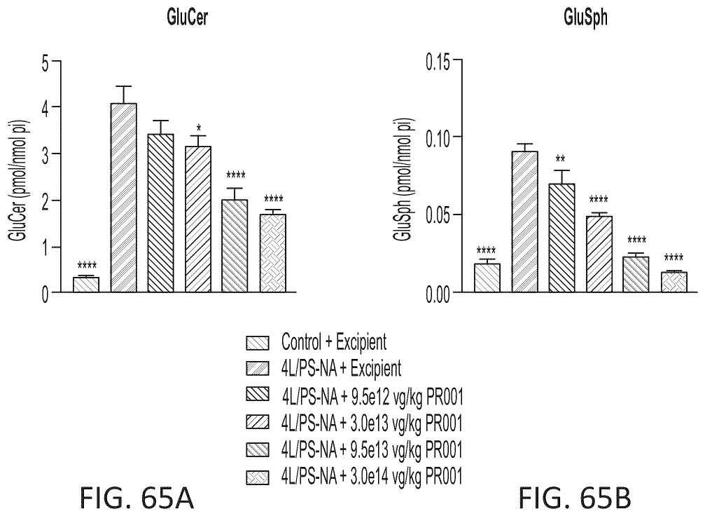

4L/PS-NA mice showed glycolipid accumulation in the liver which was reduced by PR001 treatment (FIG. 64A; FIG. 64B). 4L/PS-NA mice showed glycolipid accumulation in the brain which was reduced by PR001 treatment (FIG. 65A; FIG. 65B).

### Example 15: Studies of rAAVs Encoding Inhibitory RNA Targeting α-Synuclein

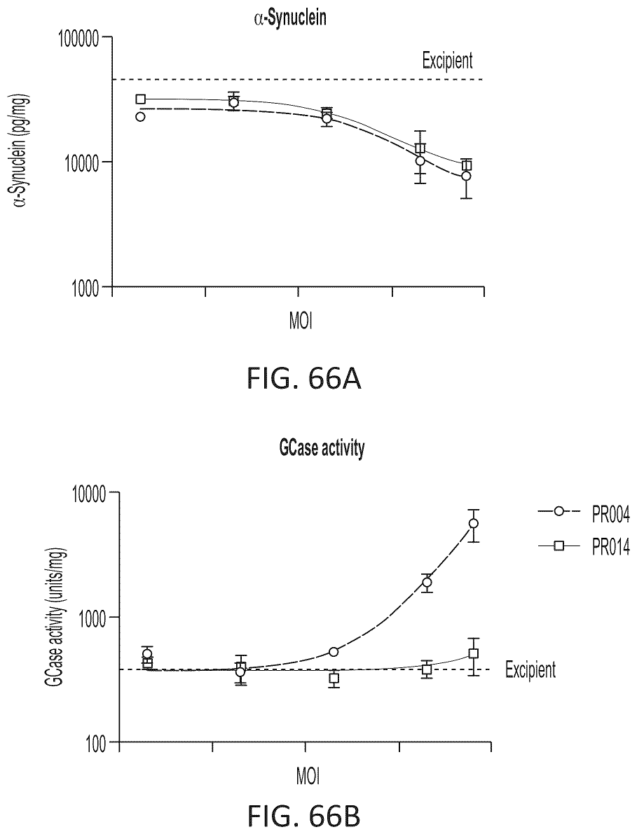

HeLa cells were transduced with PR004 or PR014 at several multiplicities of infection (MOI). Both PR004 and PR014 decreased α-Synuclein protein levels in a dose-dependent manner (FIG. 66A). PR004 increased GCase activity in a dose-dependent manner (FIG. 66B).

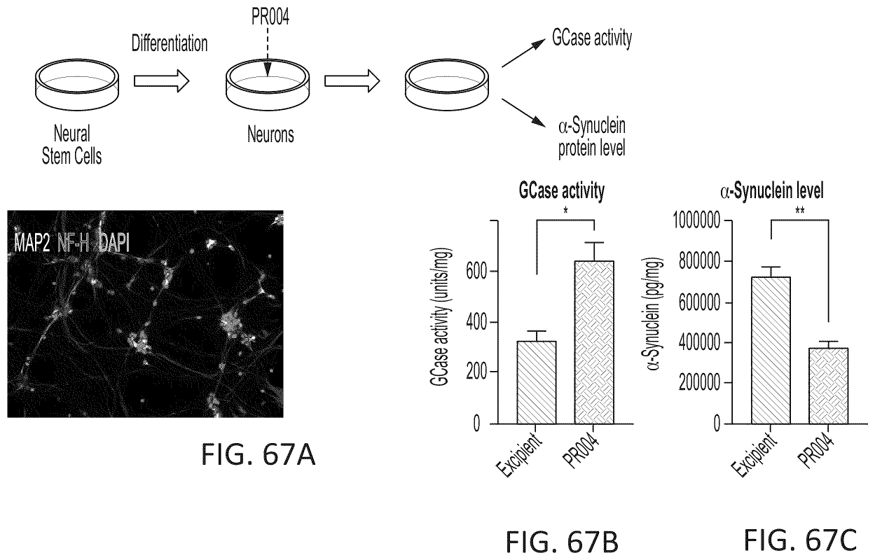

PR004 efficacy was assessed in neuronal cultures from Parkinson's disease patient-derived induced pluripotent stem cells (iPSCs). Induced pluripotent stem cells derived from a Parkinson's disease patient with a SNCA triplication were differentiated into neurons (FIG. 67A). Neurons transduced with PR004 had increased GCase activity (FIG. 67B) and decreased α-Synuclein protein level (FIG. 67C).

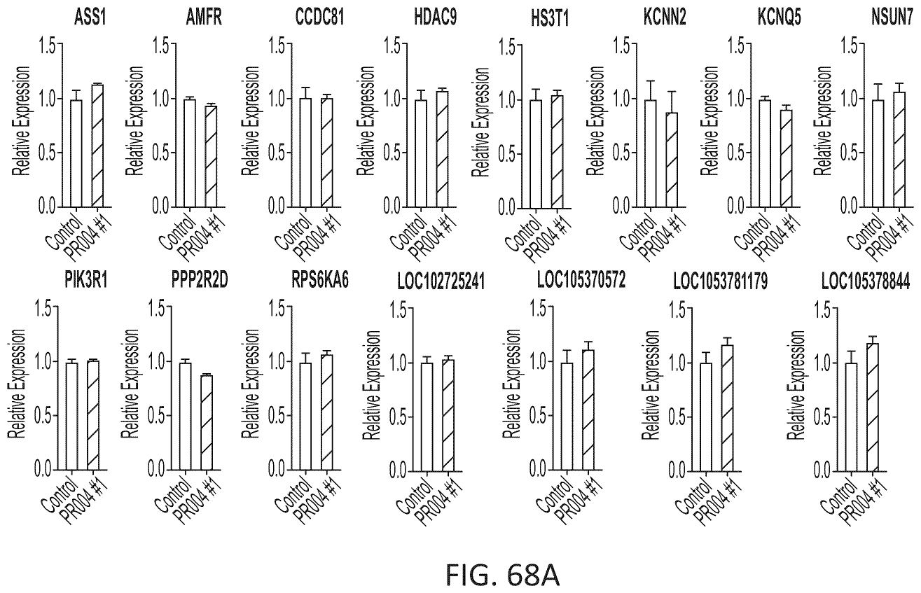

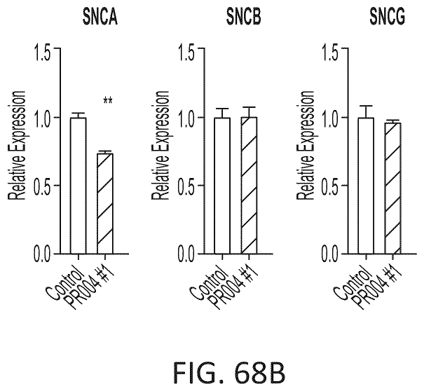

No off-target effects of the PR004 rAAV vector were observed. Off-target effects of shRNA targeting SNCA from the PR004 vector were assessed in HEK293 cells by qRT-PCR. The expression of the 15 genes most similar in sequence to the target region of SNCA was evaluated (FIG. 68A). The expression of SNCA family members beta- and gamma-synuclein (SNCB and SNCG, respectively) was also evaluated (FIG. 68B). mRNA levels of these genes were not affected by PR004.

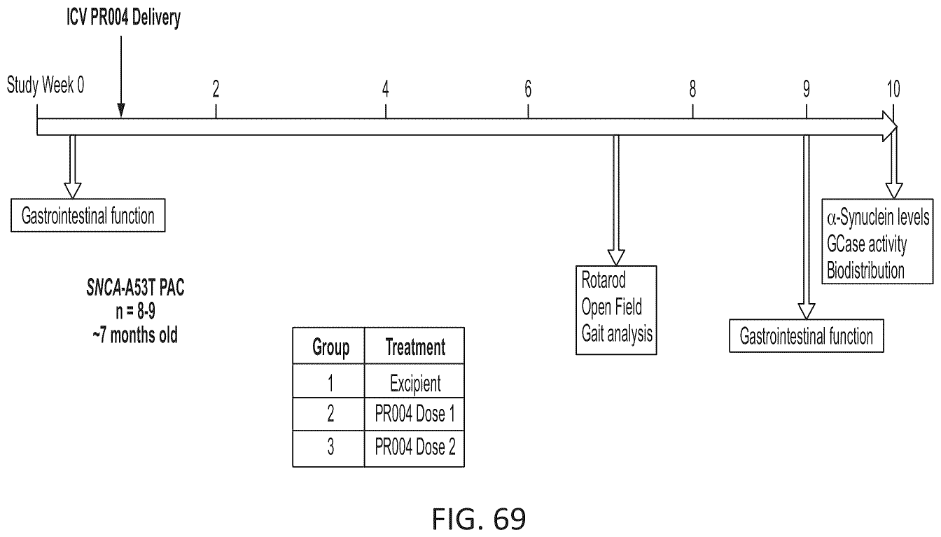

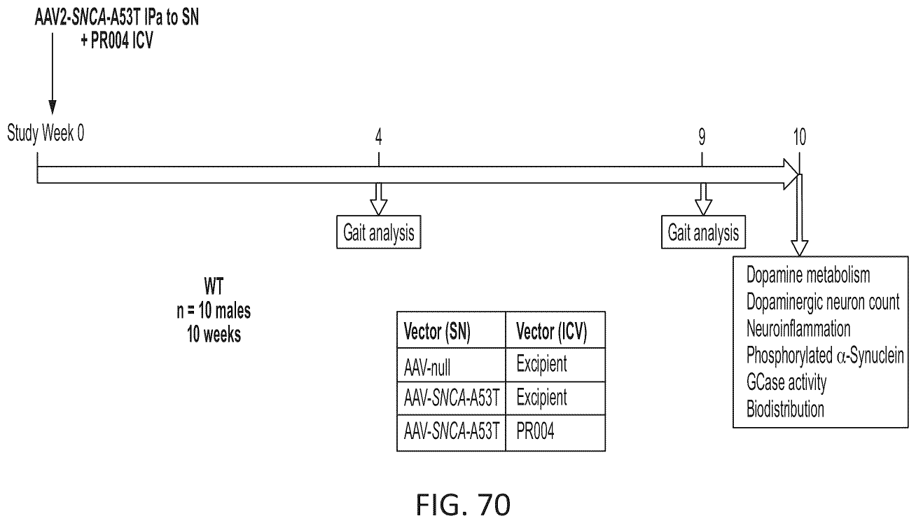

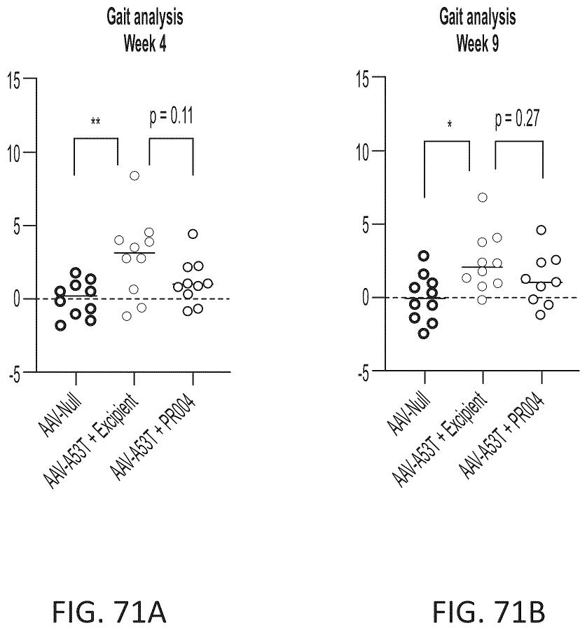

PR004 efficacy was assessed in the AAV2-SNCA-A53T AAV mouse model of Parkinson's disease (FIG. 69; FIG. 70). An AAV2 encoding human SNCA with the A53T mutation is directly injected into the substantia nigra of adult wild type mice. Starting at 4 weeks after injection, animals exhibit gait abnormalities, changes in dopamine metabolism, loss of dopaminergic neurons, neuroinflammation, and phosphorylated α-Synuclein expression. Automated kinematic gait analysis (MotoRater) was performed 4 weeks (FIG. 71A) and 9 weeks (FIG. 71B) after PR004 intracerebroventricular injection. A trend of a PR004 treatment effect was observed at both timepoints.

This Application incorporates by reference the contents of the following documents in their entirety: International PCT Application Publication No. WO 2019/070894; International PCT Application Publication No. WO 2019/070891; U.S. Provisional Application Ser. No. 62/567,311, filed Oct. 3, 2017, entitled “GENE THERAPIES FOR LYSOSOMAL DISORDERS”; 62/567,319, filed Oct. 3, 2017, entitled “GENE THERAPIES FOR LYSOSOMAL DISORDERS”; 62/567,301, filed Oct. 3, 2018, entitled “GENE THERAPIES FOR LYSOSOMAL DISORDERS”; 62/567,310, filed Oct. 3, 2017, entitled “GENE THERAPIES FOR LYSOSOMAL DISORDERS”; 62/567,303, filed Oct. 3, 2017, entitled “GENE THERAPIES FOR LYSOSOMAL DISORDERS”; and 62/567,305, filed Oct. 3, 2017, entitled “GENE THERAPIES FOR LYSOSOMAL DISORDERS”.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

Each of the U.S. patents, U.S. patent application publications, U.S. patent applications, foreign patents, foreign patent applications and non-patent publications referred to in this application is incorporated herein by reference, in its entirety.

### Sequences

In some embodiments, an expression cassette encoding one or more gene products (e.g., a first, second and/or third gene product) comprises or consists of (or encodes a peptide having) a sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 32, 33, 34, 35, 36, 37, 38, 39, 40, 41, 42, 43, 44, 45, 46, 47, or 48. In some embodiments, a gene product is encoded by a portion (e.g., fragment) of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 32, 33, 34, 35, 36, 37, 38, 39, 40, 41, 42, 43, 44, 45, 46, 47, or 48.

### Numbered Embodiments

Notwithstanding the appended claims, the disclosure sets forth the following numbered embodiments:

1. An isolated nucleic acid comprising an expression construct encoding a Gcase protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the Gcase is encoded by a codon optimized nucleic acid sequence.

2. The isolated nucleic acid of embodiment 1, wherein the Gcase protein comprises the amino acid sequence set forth in SEQ ID NO: 14 or a portion thereof.

3. The isolated nucleic acid of embodiment 1 or 2, wherein the Gcase protein is encoded by a codon-optimized nucleic acid sequence, optionally the nucleic acid sequence set forth in SEQ ID NO: 15.

4. The isolated nucleic acid of any one of embodiments 1 to 3, wherein the modified “D” region is a “D” sequence located on the outside of the ITR relative to the expression construct.

5. The isolated nucleic acid of any one of embodiments 1 to 4, wherein the ITR comprising the modified “D” sequence is a 3′ ITR

6. The isolated nucleic acid of any one of embodiments 1 to 5, further comprising a TRY sequence, optionally wherein the TRY sequence is set forth in SEQ ID NO: 28.

7. An isolated nucleic acid comprising an expression construct encoding a prosaposin protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the prosaposin is encoded by a codon optimized nucleic acid sequence.

8. The isolated nucleic acid of embodiment 7, wherein the prosaposin protein comprises the amino acid sequence set forth in SEQ ID NO: 16 or a portion thereof.

9. The isolated nucleic acid of embodiment 7 or 8, wherein the prosaposin protein is encoded by a codon-optimized nucleic acid sequence, optionally the nucleic acid sequence set forth in SEQ ID NO: 17.

10. The isolated nucleic acid of any one of embodiments 7 to 9, wherein the modified “D” region is a “D” sequence located on the outside of the ITR relative to the expression construct.

11. The isolated nucleic acid of any one of embodiments 7 to 10, wherein the ITR comprising the modified “D” sequence is a 3′ ITR.

12. The isolated nucleic acid of any one of embodiments 7 to 11, further comprising a TRY sequence, optionally wherein the TRY sequence is set forth in SEQ ID NO: 28.

13. An isolated nucleic acid comprising an expression construct encoding a SCARB2 protein flanked by two adeno-associated virus (AAV) inverted terminal repeats (ITRs), wherein
(i) at least one of the ITRs comprises a modified “D” region relative to a wild-type AAV2 ITR (SEQ ID NO: 29); and/or
(ii) the SCARB2 is encoded by a codon optimized nucleic acid sequence.

14. The isolated nucleic acid of embodiment 13, wherein the SCARB2 protein comprises the amino acid sequence set forth in SEQ ID NO: 18 or a portion thereof.

15. The isolated nucleic acid of embodiment 13 or 14, wherein the SCARB2 protein is encoded by a codon-optimized nucleic acid sequence or the nucleic acid sequence set forth in SEQ ID NO: 19.

16. The isolated nucleic acid of any one of embodiments 13 to 15, wherein the modified “D” region is a “D” sequence located on the outside of the ITR relative to the expression construct.

17. The isolated nucleic acid of any one of embodiments 13 to 16, wherein the ITR comprising the modified “D” sequence is a 3′ ITR.

18. The isolated nucleic acid of any one of embodiments 13 to 17, further comprising a TRY sequence, optionally wherein the TRY sequence is set forth in SEQ ID NO: 28.

19. An isolated nucleic acid comprising an expression construct encoding a first gene product and a second gene product, wherein each gene product independently is selected from the gene products, or portions thereof, set forth in Table 1.

20. The isolated nucleic acid of embodiment 19, wherein the first gene product is a Gcase protein, or a portion thereof.

21. The isolated nucleic acid of embodiment 19 or embodiment 20, wherein the second gene product is LIMP2 or a portion thereof, or Prosaposin or a portion thereof.

22. The isolated nucleic acid of any one of embodiments 19 to 21, further encoding an interfering nucleic acid (e.g., shRNA, miRNA, dsRNA, etc.), optionally wherein the interfering nucleic acid inhibits expression of α-Syn.

23. The isolated nucleic acid of any one of embodiments 19 to 22, further comprising one or more promoters, optionally wherein each of the one or more promoters is independently a chicken-beta actin (CBA) promoter, a CAG promoter, a CD68 promoter, or a JeT promoter.

24. The isolated nucleic acid of any one of embodiments 19 to 23, further comprising an internal ribosomal entry site (IRES), optionally wherein the IRES is located between the first gene product and the second gene product.

25. The isolated nucleic acid of any one of embodiments 19 to 23, further comprising a self-cleaving peptide coding sequence, optionally wherein the self-cleaving peptide is T2A.

26. The isolated nucleic acid of any one of embodiments 19 to 24, wherein the expression construct comprises two adeno-associated virus (AAV) inverted terminal repeat (ITR) sequences flanking the first gene product and the second gene product, optionally wherein one of the ITR sequences lacks a functional terminal resolution site.

27. The isolated nucleic acids of embodiment 26, wherein at least one of the ITRs comprises a modified “D” region relative to a wild-type AAV2 ITR (SEQ ID NO: 29).

28. The isolated nucleic acid of embodiment 27, wherein the modified “D” region is a “D” sequence located on the outside of the ITR relative to the expression construct.

29. The isolated nucleic acid of embodiment 27 or 28, wherein the ITR comprising the modified “D” sequence is a 3′ ITR.

30. The isolated nucleic acid of any one of embodiments 27 to 29, further comprising a TRY sequence, optionally wherein the TRY sequence is set forth in SEQ ID NO: 28.

31. The isolated nucleic acid of any one of embodiments 1 to 30 having the sequence set forth in any one of SEQ ID NOs: 1 to 12, 14, 16, and 18.

32. A vector comprising the isolated nucleic acid of any one of embodiments 1 to 31.

33. The vector of embodiment 32, wherein the vector is a plasmid.

34. The vector of embodiment 32, wherein the vector is a viral vector, optionally wherein the viral vector is a recombinant AAV (rAAV) vector or a Baculovirus vector.

35. A composition comprising the isolated nucleic acid of any one of embodiments 1 to 31 or the vector of any one of embodiments 32 to 34.

36. A host cell comprising the isolated nucleic acid of any one of embodiments 1 to 31 or the vector of any one of embodiments 32 to 34.

37. A recombinant adeno-associated virus (rAAV) comprising:
(i) a capsid protein; and
(ii) the isolated nucleic acid of any one of embodiments 1 to 31, or the vector of any one of embodiments 32 to 34.

38. The rAAV of embodiment 37, wherein the capsid protein is capable of crossing the blood-brain barrier, optionally wherein the capsid protein is an AAV9 capsid protein or an AAVrh.10 capsid protein.

39. The rAAV of embodiment 37 or embodiment 38, wherein the rAAV transduces neuronal cells and non-neuronal cells of the central nervous system (CNS).

40. A method for treating a subject having or suspected of having Parkinson's disease, the method comprising administering to the subject an isolated nucleic acid of any one of embodiments 1 to 31, the vector of any one of embodiments 32 to 34, the composition of embodiment 35, or the rAAV of any one of embodiments 37 to 39.

41. The method of embodiment 40, wherein the administration comprises direct injection to the CNS of the subject, optionally wherein the direct injection is intracerebral injection, intraparenchymal injection, intrathecal injection, intra-cisterna magna injection or any combination thereof.

42. The method of embodiment 41, wherein the direct injection to the CNS of the subject comprises convection enhanced delivery (CED).

43. The method of any one of embodiments 40 to 42, wherein the administration comprises peripheral injection, optionally wherein the peripheral injection is intravenous injection.

44. A method for treating a subject having Type 2 Gaucher disease or Type 3 Gaucher disease, the method comprising administering to the subject a recombinant adeno-associated virus (rAAV) comprising:
(i) a rAAV vector comprising a nucleic acid comprising an expression construct comprising a promoter operably linked to a transgene insert encoding a glucocerebrosidase (Gcase) protein, wherein the transgene insert comprises the nucleotide sequence of SEQ ID NO: 15; and
(ii) an AAV9 capsid protein.

45. The method of embodiment 44, wherein the rAAV is administered to the subject at a dose ranging from about 5×10¹⁰ vg/g brain to about 5×10¹¹ vg/g brain.

46. The method of embodiment 44, wherein the rAAV is administered to the subject at a dose of about 1.3×10¹¹ vg/g brain.

47. A method for treating a subject having Parkinson's disease with a glucocerebrosidase-1 (GBA1) mutation, the method comprising administering to the subject a recombinant adeno-associated virus (rAAV) comprising:
(i) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and
(ii) an AAV9 capsid protein.

47. The method of embodiment 46, wherein the rAAV is administered to the subject at a dose ranging from about 5×10¹³ vg to about 5×10¹⁴ vg.

48. The method of embodiment 46, wherein the rAAV is administered to the subject at a dose of about 1×10¹⁴ vg or about 2×10¹⁴ vg.

49. The method of any one of embodiments 44-48, wherein the rAAV is administered via a suboccipital injection into the cisterna magna.

50. A method for treating a subject having Type 1 Gaucher disease, the method comprising administering to the subject a recombinant adeno-associated virus (rAAV) comprising:
(i) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and
(ii) an AAV9 capsid protein.

51. The method of embodiment 50, wherein the rAAV is administered to the subject at a dose ranging from about 5×10¹³ vg to about 5×10¹⁴ vg.

52. The method of embodiment 50 or 51, wherein the rAAV is administered intravenously.

53. A method for treating a subject having a synucleinopathy or parkinsonism, the method comprising administering to the subject a recombinant adeno-associated virus (rAAV) comprising:
(i) a rAAV vector comprising a nucleic acid comprising an expression construct comprising a transgene comprising
   (a) a Gcase protein coding sequence comprising the nucleotide sequence of SEQ ID NO: 15; and
   (b) an inhibitory nucleic acid coding sequence comprising the nucleotide sequence of SEQ ID NO: 20; and
(ii) an AAV9 capsid protein.

54. A method for treating a subject having a synucleinopathy or parkinsonism, the method comprising administering to the subject a recombinant adeno-associated virus (rAAV) comprising:
(i) a rAAV vector comprising a nucleic acid comprising an expression construct comprising a transgene comprising an inhibitory nucleic acid coding sequence comprising the nucleotide sequence of SEQ ID NO: 20; and
(ii) an AAV9 capsid protein.

55. The method of embodiment 53 or 54, wherein the synucleinopathy or parkinsonism is multiple system atrophy, Parkinson's disease, Parkinson's disease with GBA1 mutation, Lewy body disease, dementia with Lewy bodies, dementia with Lewy bodies with GBA1 mutation, progressive supranuclear palsy, or corticobasal syndrome.

56. The method of any one of embodiments 44-55, wherein the promoter is a chicken beta actin (CBA) promoter.

57. The method of any one of embodiments 44-56, wherein the rAAV vector further comprises a cytomegalovirus (CMV) enhancer.

58. The method of any one of embodiments 44-57, wherein the rAAV vector further comprises a Woodchuck Hepatitis Virus Posttranscriptional Regulatory Element (WPRE).

59. The method of any one of embodiments 44-58, wherein the rAAV vector further comprises a Bovine Growth Hormone polyA signal tail.

60. The method of any one of embodiments 44-59, wherein the nucleic acid comprises two adeno-associated virus inverted terminal repeats (ITR) sequences flanking the expression construct.

61. The method of embodiment 60, wherein each ITR sequence is a wild-type AAV2 ITR sequence.

62. The method of any one of embodiments 44-61, wherein the rAAV vector further comprises a TRY region between the 5′ ITR and the expression construct, wherein the TRY region comprises SEQ ID NO: 28.

63. A method for treating a subject having Type 2 Gaucher disease or Type 3 Gaucher disease, the method comprising administering to the subject a rAAV comprising:
(i) a rAAV vector comprising a nucleic acid comprising, in 5′ to 3′ order:
   (a) an AAV2 ITR;
   (b) a CMV enhancer;
   (c) a CBA promoter;
   (d) a transgene insert encoding a Gcase protein, wherein the transgene insert comprises the nucleotide sequence of SEQ ID NO: 15;
   (e) a WPRE;
   (f) a Bovine Growth Hormone polyA signal tail; and
   (g) an AAV2 ITR; and
(ii) an AAV9 capsid protein;
   wherein the rAAV is administered to the subject at a dose ranging from about 5×10¹⁰ vg/g brain to about 5×10¹¹ vg/g brain.

64. A method for treating a subject having Parkinson's disease with a GBA1 mutation, the method comprising administering to the subject a rAAV comprising:
(i) a rAAV vector comprising a nucleic acid comprising, in 5′ to 3′ order:
   (a) an AAV2 ITR;
   (b) a CMV enhancer;
   (c) a CBA promoter;
   (d) a transgene insert encoding a Gcase protein, wherein the transgene insert comprises the nucleotide sequence of SEQ ID NO: 15;
   (e) a WPRE;
   (f) a Bovine Growth Hormone polyA signal tail; and
   (g) an AAV2 ITR; and
(ii) an AAV9 capsid protein;
   wherein the rAAV is administered to the subject at a dose ranging from about 5×10¹³ vg to about 5×10¹⁴ vg.

65. The method of embodiment 63 or 64, wherein the rAAV is administered via a suboccipital injection into the cisterna magna.

66. The method of any one of embodiments 44-52 and 63-65, wherein the rAAV is administered in a formulation comprising about 20 mM Tris, pH 8.0, about 1 mM MgCl₂, about 200 mM NaCl, and about 0.001% w/v poloxamer 188.

67. A pharmaceutical composition comprising
(i) a rAAV comprising:
   (a) a rAAV vector comprising a nucleic acid comprising an expression construct comprising a promoter operably linked to a transgene insert a Gcase protein, wherein the transgene insert comprises the nucleotide sequence of SEQ ID NO: 15; and
   (b) an AAV9 capsid protein; and
(ii) about 20 mM Tris, pH 8.0,
(iii) about 1 mM MgCl₂,
(iv) about 200 mM NaCl, and
(v) about 0.001% w/v poloxamer 188.

68. A rAAV comprising:
(i) a rAAV vector comprising a nucleic acid comprising an expression construct comprising a promoter operably linked to a transgene insert encoding a Gcase protein, wherein the transgene insert comprises the nucleotide sequence of SEQ ID NO: 15; and
(ii) an AAV9 capsid protein,
   for use in a method of treating Type 1 Gaucher disease, Type 2 Gaucher disease, Type 3 Gaucher disease or Parkinson's disease with a GBA1 mutation in a subject.

69. A rAAV comprising:
(i) a rAAV vector comprising a nucleic acid comprising an expression construct comprising a promoter operably linked to a transgene insert comprising:
   (a) a Gcase protein coding sequence comprising the nucleotide sequence of SEQ ID NO: 15; and
   (b) an inhibitory nucleic acid coding sequence comprising the nucleotide sequence of SEQ ID NO: 20; and
(ii) an AAV9 capsid protein,
   for use in a method of treating a synucleinopathy or parkinsonism in a subject.

70. A rAAV comprising:
(i) a rAAV vector comprising a nucleic acid comprising an expression construct comprising a promoter operably linked to a transgene insert comprising an inhibitory nucleic acid coding sequence comprising the nucleotide sequence of SEQ ID NO: 20; and
(ii) an AAV9 capsid protein,
   for use in a method of treating a synucleinopathy or parkinsonism in a subject.

TABLE 14
Summary of Nonclinical In Vivo Pharmacology (Efficacy) Studies

| Study Number | Objective | Status | Results |
|---|---|---|---|
| PRV-2017-001 | Validate CBE mouse as a | Completed | 25 mg/kg CBE recapitulates core features of GCase deficiency |
| | model of GCase | | Glycolipid (aggregate GluSph and galactosylsphingosine [GalSph]) |
| | deficiency | | accumulation relative to controls |
| | | | Motor deficits in rotarod assay |
| PRV-2018-002 | Demonstrate efficacy of | Completed | Broad vector genome biodistribution of PR001B |
| | highest possible dose of | | Increase in GCase activity correlated with reduction of glycolipid substrate |
| | PR001Ba in CBE mouse | | accumulation |
| | | | Improved behavioral performance on rotarod assay |
| | | | No PR001B-related adverse effects observed |
| PRV-2018-005 | Determine efficacious doses | Completed | Broad vector genome biodistribution of PR001A |
| | of ICV PR001A in CBE | | Increase in GCase activity and reduction in abnormal glycolipid substrate |
| | mouse model | | accumulation |
| | | | Reduction of astrogliosis and microgliosis |
| | | | Improvement of motor behavior deficits |
| | | | No PR001A-related adverse effects observed |
| PRV-2018-007 | Long-term (6 month) | Completed | Vector genome persistence, durable increased GCase activity, and reduction in |
| | persistence of ICV PR001A | | glycolipids 6 months post treatment |
| | in CBE mouse model | | No PR001A-related adverse effects observed |
| PRV-2018-008 | Additional dose-ranging ICV | Completed | Broad vector genome biodistribution of PR001A |
| | PR001A in CBE mouse | | Increase in cortical GCase activity and reduction in abnormal glycolipid substrate |
| | model | | accumulation |
| | | | No PR001A-related adverse effects observed |
| PRV-2018-025 | Further dose-ranging ICV | Ongoing | Cortical vector genome biodistribution of PR001A |
| | PR001A in CBE mouse | | Increase in cortical GCase activity and reduction in abnormal glycolipid substrate |
| | model | | accumulation |
| | | | Improvement of motor behavior deficits |
| PRV-2018-006 | Demonstrate efficacy of ICV | Completed | Broad vector genome biodistribution of PR001A |
| | PR001A in 4L/PS-NA | | Increase GCase activity in CNS and periphery associated with reduction of |
| | mouse model | | glycolipid substrate accumulation |
| | | | Trend towards improved motor behavior |
| | | | Reduction in accumulation of insoluble cc-Synuclein |
| | | | No PR001A-related adverse effects observed |
| PRV-2018-011 | Dose-ranging ICV PR001A | Completed | Broad vector genome biodistribution of PR001A |
| | in 4L/PS-NA genetic mouse | | Increase in GCase activity and reduction in abnormal glycolipid substrate |
| | model | | accumulation |
| | | | Improvement of motor behavior deficits |
| | | | No PR001A-related adverse effects observed |
| PRV-2018-019 | Effect of ICV PR001A on α- | Ongoing | Cortical vector genome biodistribution of PR001A and reduction in glycolipids |
| | Synuclein in transgenic mice | | Reduction in accumulation of insoluble α-Synuclein and ratio of insoluble to |
| | treated with CBE | | soluble accumulation of insoluble α-Synuclein |
| | | | No PR001A-related adverse effects observed |
| PRV-2019-001 | Effect of ICV PR001A on α- | Ongoing | Cortical vector genome biodistribution of PR001A and increase in cortical GCase |
| | Synuclein in transgenic mice | | activity |
| | treated with CBE | | No PR001A-related adverse effects observed |

Abbreviations:
CBE, conduritol-β-epoxide;
CNS, central nervous system;
GCase, glucocerebrosidase;
GluSph, glucosylsphingosine;
ICV, intracerebroventricular;
vg, vector genome.
^{a}PR001B is a version of PR001A with an altered D domain; PR001A and PR001B are otherwise identical.

TABLE 15
Summary of Safety Evaluations in Mouse Efficacy Studies of PR001A

| Study | Study | Administration | | Dose | Total PR001A | Injection | Necropsy Time |
|---|---|---|---|---|---|---|---|
| Purpose | Number | Route | Species | (vg/g brain) | Dose (vg) | Volume(s) (μl) | Point^{a} |
| Efficacy with | PRV-2018-005 | ICV | CBE-injected | 1.3 × 10¹⁰ | 2.0 × 10⁹ | 4 | Days 35-37 |
| select safety | | | C57BL/6J mice | 4.2 × 10¹⁰ | 6.2 × 10⁹ | | |
| endpoints | | | | 1.3 × 10¹¹ | 2.0 × 10¹⁰ | | |
| Efficacy with | PRV-2018-006 | ICV | 4L/PS-NA mice | 3.7 × 10¹⁰ | 1.5 × 10¹⁰ | 3 | Week 15 |
| select safety | | | | | | | |
| endpoints | | | | | | | |

Abbreviations:
CBE, conduritol-β-epoxide;
ICV, intracerebroventricular;
vg, vector genome.
^{a}Post-PR001A treatment

TABLE 16
Summary of Safety Evaluations in NHP Safety Studies of PR001A

| Study | Study | Administration | | Dose | Total PR001A | Injection | Necropsy Time |
|---|---|---|---|---|---|---|---|
| Purpose | Number | Route | Species | (vg/g brain) | Dose (vg) | Volume(s) (μl) | Point^{a} |
| Pilot non-GLP | PRV-2018-015 | ICM | Cynomolgus | 2.0 × 10¹¹ | 1.47 × 10¹³ | 500 | Day 18 |
| toxicology | | ICM + IPa | monkeys | 2.1 × 10¹¹ | 1.53 × 10¹³ | 520 | |
| | | (midbrain) | | | | | |
| GLP toxicology | PRV-2018-016 | ICM | Cynomolgus | 6.2 × 10¹⁰ | 4.6 × 10¹² | 1200 | Days 7, 30, 183 |
| | | | monkeys | 2.3 × 10¹¹ | 1.7 × 10¹³ | | |
| Non-GLP | PRV-2019-005 | ICM | Cynomolgus | 7.0 × 10¹¹ | 5.2 × 10¹³ | 1200 | Days 30, 90 |
| toxicology | | | monkeys | | | | |

Abbreviations:
GLP, Good Laboratory Practice;
ICM, intra-cisterna magna;
IPa, intraparenchymal;
NHP, nonhuman primate;
vg, vector genome.

What is claimed is:

1. A pharmaceutical composition comprising
(i) a recombinant adeno-associated virus (rAAV) comprising:
(a) a rAAV vector comprising a nucleic acid comprising an expression construct comprising a promoter operably linked to a transgene insert encoding a glucocerebrosidase (Gcase) protein, wherein the transgene insert comprises the nucleotide sequence of SEQ ID NO: 15; and
(b) an AAV9 capsid protein; and
(ii) about 20 mM Tris, pH 8.0,
(iii) about 1 mM MgCl₂,
(iv) about 200 mM NaCl, and
(v) about 0.001% w/v poloxamer 188.

2. The pharmaceutical composition of claim 1, wherein the promoter is a chicken beta actin (CBA) promoter.

3. The pharmaceutical composition of claim 1, wherein the rAAV vector further comprises a cytomegalovirus (CMV) enhancer.

4. The pharmaceutical composition of claim 1, wherein the rAAV vector further comprises a Woodchuck Hepatitis Virus Posttranscriptional Regulatory Element (WPRE).

5. The pharmaceutical composition of claim 1, wherein the rAAV vector further comprises a Bovine Growth Hormone polyA signal tail.

6. The pharmaceutical composition of claim 1, wherein the nucleic acid comprises two adeno-associated virus inverted terminal repeat (ITR) sequences flanking the expression construct, wherein the first ITR sequence is a 5′ ITR, and the second ITR sequence is a 3′ ITR.

7. The pharmaceutical composition of claim 6, wherein each of the two ITR sequences is a wild-type AAV2 ITR sequence.

8. The pharmaceutical composition of claim 6, wherein the rAAV vector further comprises a TRY region between the 5′ ITR and the expression construct, wherein the TRY region comprises SEQ ID NO: 28.

9. A pharmaceutical composition comprising:
(i) a rAAV comprising:
(a) a rAAV vector comprising a nucleic acid comprising, in 5′ to 3′ order:
an AAV2 ITR;
a CMV enhancer;
a CBA promoter;
a transgene insert encoding a Gcase protein, wherein the transgene insert comprises the nucleotide sequence of SEQ ID NO: 15;
a WPRE;
a Bovine Growth Hormone polyA signal tail; and
an AAV2 ITR; and
(b) an AAV9 capsid protein; and
(ii) about 20 mM Tris, pH 8.0,
(iii) about 1 mM MgCl₂,
(iv) about 200 mM NaCl, and
(v) about 0.001% w/v poloxamer 188.
